SEQUENCE LISTING (10 sequences)

SEQ ID NO: 1 - DNA, 2896 bp, Homo sapiens
  CDS (412)..(1389)
atgagtgtcagaagtgtgaagggtgcctgttctgaatcccagagcctcctctccctctgtgaggctggcaggtgaggaagggtttaacctcactggaaggaatccctggagctagcggctgctgaaggcgtcgaggtgtgggggcacttggacagaacagtcaggcagccgggagctctgccagctttggtgaccttgggccgggctgggagcgctgcggcgggagccggaggactatgagctgccgcgcgttgtccagagcccagcccagccctaccgcgcgcggcccggagctctgttccctggaactttgggcactgcctctgggacccctgccggccagcaggcaggatggtgcttgcctcgtgccccttggtgcccgtctgctgatgtgcccagcctgtgcccgccatgccgccctccatctcagctttccaggccgcctacatcggcatcgaggtgctcatcgccctggtctctgtgcccgggaacgtgctggtgatctgggcggtgaaggtgaaccaggcgctgcgggatgccaccttctgcttcatcgtgtcgctggcggtggctgatgtggccgtgggtgccctggtcatccccctcgccatcctcatcaacattgggccacagacctacttccacacctgcctcatggttgcctgtccggtcctcatcctcacccagagctccatcctggccctgctggcaattgctgtggaccgctacctccgggtcaagatccctctccggtacaagatggtggtgaccccccggagggcggcggtggccatagccggctgctggatcctctccttcgtggtgggactgacccctatgtttggctggaacaatctgagtgcggtggagcgggcctgggcagccaacggcagcatgggggagcccgtgatcaagtgcgagttcgagaaggtcatcagcatggagtacatggtctacttcaacttctttgtgtgggtgctgcccccgcttctcctcatggtcctcatctacctggaggtcttctacctaatccgcaagcagctcaacaagaaggtgtcggcctcctccggcgacccgcagaagtactatgggaaggagctgaagatcgccaagtcgctggccctcatcctcttcctctttgccctcagctggctgcctttgcacatcctcaactgcatcaccctcttctgcccgtcctgccacaagcccagcatccttacctacattgccatcttcctcacgcacggcaactcggccatgaaccccattgtctatgccttccgcatccagaagttccgcgtcaccttccttaagatttggaatgaccatttccgctgccagcctgcacctcccattgacgaggatctcccagaagagaggcctgatgactagaccccgccttccgctcccaccagcccacatccagtggggtctcagtccagtcctcacatgcccgctgtcccaggggtctccctgagcctgccccagctgggctgttggctgggggcatgggggaggctctgaagagatacccacagagtgtggtccctccactaggagttaactaccctacacctctgggccctgcaggaggcctgggagggcaagggtcctacggagggaccaggtgtctagaggcaacagtgttctgagcccccacctgcctgaccatcccatgagcagtccagagcttcagggctgggcaggtcctggggaggctgagactgcagaggagccacctgggctgggagaaggtgcttgggcttctgcggtgaggcaggggagtctgcttgtcttagatgttggtggtgcagccccaggaccaagcttaaggagaggagagcatctgctctgagacggatggaaggagagaggttgaggatgcactggcctgttctgtaggagagactggccagaggcagctaaggggcaggaatcaaggagcctccgttcccacctctgaggactctggaccccaggccataccaggtgctagggtgcctgctctccttgccctgggccagcccaggattgtacgtgggagaggcagaaagggtaggttcagtaatcatttctgatatttgctggagtgctggctccacgccctggggagtgagcttggtgcggtaggtgctggcctcaaacagccacgaggtggtagctctgagccctccttcttgccctgagctttccggggaggagccttggagtgtaattacctgtcatctgggccaccagctccactggcctgcccgttgccgggcctggactgtcctaggtgaccccatctctgctgcttctgggcctgatggagaggagaacactagacatgccaactcgggagcattctgcctgcctgggaacggggtggacgagggagtgtctgtaaggactcagtgttgactgtaggcgcccctggggtgggtttagcaggctgcagcaggcagaggagagtacccccctgagagcatgtgggggaaggccttgctgtcatgtgaatccctcaatacccctagtatctggctgggttttcaggggctttggaagctctgttgcaggtgtccgggggtctaggactttagggatctggggaaggaccaacccatgccctgccaagcctggagcccctgtgttggggggcaaggtgggggagcctggagcccctgtgtgggagggcgaggcgggggagcctggagcccctgtgtgggagggcgaggcgggggatcctggagcccctgtgtcggggggcgagggaggggaggtggccgtcgagttgaccttctgaacatgagtgtcaactccaggacttgcttccaagcccttccctctgttggaaattgggtgtgccctggctcccaagggaggcccatgtgactaataaaaaactgtgaaccct

SEQ ID NO: 2 - DNA, 2241 bp, Homo sapiens
  CDS (768)..(1721)
atctttgctgcaaaggctgggtatcggctgtgctcagcaaagcgtcaactcgtgcaagaacttagcaggaatagttctggctaaggttaggaggctgccaccaaagtctcttttttgttcctctgcttctcccgtttgcctccttatcatgagatctttttgctaagctggcagaaagattgcatagtcagtgcttccagctctgctcccacctgatcctgcactgtcctctggtccctgaatgaatgaactctgatacccaatcttgtctcgagccttctctatgccactcatggctcctcttctgctctttccatctttttgctgagagttctgagctctgtacttcctcttggcccatctcacttcctgaaacacccctgaagagggttgcttatcttgatggaactcaaaaagccaaaaagctgcaggcagaggcgttgaggacatctgtttggggaactaagagcagcagcactttcagattcagtccatatagagctgtcctacagcattctggaaacttgaggatgtgcggtgcataaaggggctggaagtgacccacctgtgatgagccctttctaaggagaagggtttccaagagatcaccccaccagaaaagggtaggaatgagcaagttgggaattttagactgtcactgcacatggacctctgggaagacgtctggcgagagctaggcccactggccctacagacggatcttgctggctcacctgtccctgtggaggttcccctgggaaggcaagatgcccaacaacagcactgctctgtcattggccaatgttacctacatcaccatggaaattttcattggactctgcgccatagtgggcaacgtgctggtcatctgcgtggtcaagctgaaccccagcctgcagaccaccaccttctatttcattgtctctctagccctggctgacattgctgttggggtgctggtcatgcctttggccattgttgtcagcctgggcatcacaatccacttctacagctgcctttttatgacttgcctactgcttatctttacccacgcctccatcatgtccttgctggccatcgctgtggaccgatacttgcgggtcaagcttaccgtcagatacaagagggtcaccactcacagaagaatatggctggccctgggcctttgctggctggtgtcattcctggtgggattgacccccatgtttggctggaacatgaaactgacctcagagtaccacagaaatgtcaccttcctttcatgccaatttgtttccgtcatgagaatggactacatggtatacttcagcttcctcacctggattttcatccccctggttgtcatgtgcgccatctatcttgacatcttttacatcattcggaacaaactcagtctgaacttatctaactccaaagagacaggtgcattttatggacgggagttcaagacggctaagtccttgtttctggttcttttcttgtttgctctgtcatggctgcctttatctatcatcaactgcatcatctactttaatggtgaggtaccacagcttgtgctgtacatgggcatcctgctgtcccatgccaactccatgatgaaccctatcgtctatgcctataaaataaagaagttcaaggaaacctaccttttgatcctcaaagcttgtgtggtctgccatccctctgattctttggacacaagcattgagaagaattctgagtagttatccatcagagatgactctgtctcattgaccttcagattccccatcaacaaacacttgagggcctgtatgcctgggccaagggatttttacatccttgattacttccactgaggtgggagcatctccagtgctccccaattatatctcccccactccactactctcttcctccacttcatttttcccttgtcctttctctctaattcagtgttttggaggcctgacttggggacaacgtattattgatattattgtctgttttccttcttcccaatagaagaataagtcatggagcctgaagggtgcctagttgacttactgacaaaaggctccagttgggctgaacatgtgtgtggtggtgactcatttccataccattgtggaattgagcagagaacctgctctcggaggatgcctaggagatgttgggaacagaaaaaataaactgagtttaagggggacttaaactgctgaattcacctgtggatgtttttgagtaaataaaagctaatag

SEQ ID NO: 3 - protein, 318 aa, Homo sapiens
MPNNSTALSLANVTYITMEIFIGLCAIVGNVLVICVVKLNPSLQTTTFYFIVSLALADIAVGVLVMPLAIVVSLGITIHFYSCLFMTCLLLIFTHASIMSLLAIAVDRYLRVKLTVRYKRVTTHRRIWLALGLCWLVSFLVGLTPMFGWNMKLTSEYHRNVTFLSCQFVSVMRMDYMVYFSFLTWIFIPLVVMCAIYLDIFYIIRNKLSLNLSNSKETGAFYGREFKTAKSLFLVLFLFALSWLPLSIINCIIYFNGEVPQLVLYMGILLSHANSMMNPIVYAYKIKKFKETYLLILKACVVCHPSDSLDTSIEKNSE

SEQ ID NO: 4 - RNA, 49 nt, Homo sapiens
gcaguccagcgcuucagggcugggcagguccuggggaggcugagacugc

SEQ ID NO: 5 - RNA, 49 nt, Homo sapiens
gcaguccagagcuucagggcugggcagguccuggggaggcugagacugc

SEQ ID NO: 6 - RNA, 51 nt, Homo sapiens
gcucugagcccuccuucuugcccugagcuuuccggggaggagccuuggagu

SEQ ID NO: 7 - RNA, 50 nt, Homo sapiens
gcucugagcccuccuucuugcccugagcuuuccggggaggagccuggagu

SEQ ID NO: 8 - DNA, 18 bp, Artificial Sequence
  Description of Artificial Sequence Synthetic primer
acccggaggtagaggtcc

SEQ ID NO: 9 - DNA, 18 bp, Artificial Sequence
  Description of Artificial Sequence Synthetic primer
atcgccctggtctctgtg

SEQ ID NO: 10 - protein, 326 aa, Homo sapiens
MPPSISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPSCHKPSILTYIAIFLTHGNSAMNPIVYAFRIQKFRVTFLKIWNDHFRCQPAPPIDEDLPEERPDD

(19) United States
(12) Patent Application Publication
(10) Pub. No.: US 2010/0047798 A1
(43) Pub. Date: Feb. 25, 2010

(54) ADENOSINE A1 AND A3 RECEPTOR GENE SEQUENCE VARIATIONS FOR PREDICTING DISEASE OUTCOME AND TREATMENT OUTCOME

(75) Inventors: Arthur M. Feldman, Wynnewood, PA (US); Zhong Tang, Cherry Hill, NJ (US)

Correspondence Address: DAVID S. RESNICK, NIXON PEABODY LLP, 100 SUMMER STREET, BOSTON, MA, 02110-2131, US

(73) Assignee: THOMAS JEFFERSON UNIVERSITY, Philadelphia, PA (US)

(21) Appl. No.: 12/514,031

(22) PCT Filed: Nov. 8, 2007
(86) PCT No.: PCT/US07/84083

§ 371 Date: May 7, 2009

Related U.S. Application Data
(60) Provisional application No. 60/857,562, filed on Nov. 8, 2006.

(51) Int. Cl. C12Q 1/68; C07H 21/04; C07K 2/00; C07K 16/00; G06F 19/00

(52) U.S. Cl. 435/6; 536/24.31; 530/300; 530/387.1; 530/387.3; 702/19

(57) ABSTRACT

The present invention relates to methods for identify subjects for responsiveness to adenosine agonist treatment. Another aspect of the present invention relates to methods to predict a relative infarct size in response to ischemia reperfusion injury. In particular, the present invention relates to methods for to identify responsiveness to adenosine agonist treatment and/or relative infarct size by identifying a sequence differences such as mutations and/or polymorphisms in the human A1 adenosine receptor (A1-AR) gene that alters the stability of the A1-AR mRNA. Other aspect of the present invention relates to methods to identify responsiveness to adenosine agonist treatment and/or relative infarct size by identifying a sequence differences, such as mutations and/or polymorphisms in the human A3 adenosine receptor (A3-AR) gene that alters the A3-AR protein function. Other aspect of the present invention also relate to kits and assays to detect sequence differences in the human A1 adenosine receptor (A1-AR) gene and/or A3 adenosine receptor (A3-AR) gene.

### CROSS REFERENCE TO RELATED APPLICATIONS

This application is a 371 National Phase Entry Application of co-pending International Application PCT/US2007/084083 filed Nov. 8, 2007, which designated the U.S., and claims the benefit under 35 U.S.C. 119(e) of U.S. Provisional Application Ser. No. 60/857,562 filed on Nov. 8, 2006, the contents of which are incorporated herein by reference in their entirety.

### GOVERNMENT SUPPORT

This invention was made with Government Support under Grant No. RO1 HL69009-02 and No. UO1 HL 69012-01 awarded by the National Institutes for Health (NIH). The Government of the United States has certain rights in the invention.

### FIELD

The present invention describes the use of genetic variance information of genes involved in the adenosine receptor pathways. In particular identifying subjects with a likelihood of large myocardial infarction or small myocardial infarction. Further, the present invention relates to predicting the responsiveness to adenosine receptor agonists.

### BACKGROUND

Adenosine is a ubiquitous purine nucleoside that plays a critical role in cardiac protection in the setting of ischemia-reperfusion. (Headrick J P, Am J Physiol Heart circ Physiol 285; H1797-H1818; 2003) The cardio-protective effects of adenosine are regulated by both the quantity of adenosine as well as by the number of functional adenosine receptors that are expressed on the cell surface. Four known G protein-coupled adenosine receptor (AR) subtypes (A1-, A2A-, A2B-, and A3-AR's) have been identified and are expressed in a tissue specific manner. In the heart, A1- and A3-AR activation couples with the G regulatory proteins Gi and Go to inhibit adenylyl cyclase activity and enhance an inwardly rectifying K+ current. By contrast, activation of myocardial A2A-ARs results in coupling with the stimulatory guanine-nucleotide binding protein Gs and activation of adenylyl cyclase leading to synthesis of cyclic AMP and phosphorylation of protein kinase A (PKA).

The precise role of each adenosine receptor has been controversial; however, the recent use of genetically modified mice has allowed investigators to begin to tease apart the roles of selective receptor subtypes. Genetic deletion of the A1-AR limits the ability of mouse myocardium to withstand injury during ischemia-reperfusion while over-expression of the A1-AR or the A3-AR confers enhanced tolerance to ischemia and decreased infarct size (Yang Z, Am J Physiol Heart Circ Physiol 282; H949:2002) Activation of the A2A-AR also provides cardioprotection; however, the effects appear to occur primarily in the post-ischemia period and to be mediated by modulation of inflammatory responses and a decrease in apoptosis. Thus, A1- and A3-ARs appear to reduce injury during acute ischemic injury (inflammation independent) whereas A2A-AR's exert beneficial effects during reperfusion (inflammation dependent). Activation of the A1-AR has also been shown to attenuate cardiac hypertrophy and attenuate the development of heart failure in mice with left ventricular pressure-overload (Liao Y, Circ Res 2003; 93:759-766); however, these salutary benefits are dependent on both dose and genetic background. (Funakoshi H, Circ in press)

Adenosine is an endogenous substance that interacts with four distinct G-protein coupled membrane receptors A1, A2A, A2B and A3. Through interaction with the A1-adenosine receptors (A1-AR) episodes of ischemia (brief occlusion of a coronary artery) followed by reperfusion (opening of the artery) in experimental animals protects the heart during the subsequent permanent occlusion of the artery. That this protection is seen by the fact that the size of the infarct is substantially smaller in hearts that have undergone ischemic preconditioning than in hearts that have not received any therapy before infarction. This process is mediated by the A1-adenosine receptor is seen by the fact that the infarct size is substantially smaller in hearts in which the A1-adenosine receptor is overexpressed than in hearts from wild-type animals and that the process of ischemic preconditioning cannot be demonstrated in hearts which the A1-adenosine receptor has been “knocked-out” or reduced. It is commonly believed that patients with coronary artery disease and a history of coronary ischemia have smaller infarcts than patients with their first ever coronary occlusion. However, some subjects that have a history of coronary ischemia or ischemia-reperfusion injury have a large infarct size on myocardial infarction as compared to other subjects. Similarly, some patients respond well to adenosine and/or adenosine receptor agonists for the treatment of acute coronary syndrome such as myocardial infarction and the reason for this is unknown. Therefore there is much need in the art of methods to identify patients that are responsive to adenosine and/or adenosine receptor agonists.

### SUMMARY

The present invention provides methods for screening subjects for responsiveness to adenosine agonist treatment. The invention also provides methods for screening for predicting infarct size upon ischemic injury. In some embodiments, the infarction is myocardial infarction. In particular, the invention also relates to methods for screening subjects for increased susceptibility to, or current affliction with, a disease or disorder associated with a variance (e.g. mutation or polymorphism) in the human A1 adenosine receptor (A1-AR) gene that alters the A1-AR RNA stability, for example mutations that increase the A1-AR RNA stability or destabilize A1-AR RNA, and/or variations or sequence differences that result in a decreased function of the A3 adenosine receptor (A3-AR). In one embodiment, the variance is in the 3′-untranslated region (3′UTR) of the A1 adenosine receptor (A1-AR) and/or the coding region of the A3 adenosine receptor (A3-AR). In some embodiments, the subject has coronary syndrome and coronary artery disease. In some embodiments, the disease or disorder is myocardial infarction. In other embodiments, the disease or disorder is any disease or disorder where stability of A1-AR RNA and/or dysfunction of A3-AR protein contribute to the pathology of the disease or disorder. Such diseases or disorders include but are not limited to disorders of the circulatory system, disorders in vasoconstriction, disorders in renal fluid balance and sleep disorders.

In one embodiment, the methods comprise obtaining a biological sample from a subject and screening for variations or sequence differences (e.g. changes) in the 3′UTR of the human A1-AR gene relative to a control group (e.g. wildtype, positive and/or negative control group). In other embodiments, screening is for variations or sequence differences in the human A3-AR gene or gene products relative to a control group. In other embodiments, screening is for variations or sequence differences in the 3′UTR of the human A1-AR gene and the coding region of A3-AR gene, which can be done together or separately.

In the methods of the present invention, the wild type nucleic acid sequence for the 3′UTR for A1-AR gene corresponds to nucleic acid SEQ ID NO:1. In the methods of the present invention, the wild type nucleic acid sequence for the A3-AR gene corresponds to nucleic acid SEQ ID NO:2. Therefore, in some embodiments, the methods as disclosed herein relate to detecting sequence differences in the nucleic acid sequences from a subject as compared to the nucleic acid sequences corresponding to SEQ ID NO:1 and/or SEQ ID NO:2, which correspond to the nucleic acid sequence transcripts for wild type (WT) A1-AR and/or A3-AR, respectively.

In some embodiments, the presence of particular variances or sequence differences (for example, changes, mutation, polymorphism or SNP) in the 3′UTR of the human A1-AR gene in the biological sample, as compared to the control group results in altered A1-AR RNA stability. Thus, in one embodiment, polymorphisms and/or mutations within the 3′UTR of the A1-AR gene predispose a subject to different responses to adenosine and adenosine agonists and contribute to determine infarct size. In one embodiment, variances that decrease A1-AR RNA stability indicate that a subject is likely to have a large infarct size and also an increased responsiveness to adenosine receptor agonist therapy. In these embodiments, the inventors have discovered that these variances in the 3′UTR of A1-AR decrease the stability of, or destabilize, the A1-AR RNA and thus function as “susceptibility alleles”. One such susceptibility allele is, for example, a 36 nucleotide deletion beginning at position 2683 in SEQ ID NO:1 as compared no deletion in the wildtype A1-AR nucleic sequence corresponding to SEQ ID NO:1 (e.g. negative control)). This 3′UTR A1-AR susceptibility allele, also termed as “nt2683(2777)del36” herein, is associated with a subject having a large infarct and an increased responsiveness to adenosine receptor agonist.

In an alternative embodiment, different variances (e.g. change, mutation, polymorphism or SNP) in the 3′UTR of the human A1-AR gene in the biological sample, as compared to the control group increase the A1-AR RNA stability and act as “protective alleles”. An increase in A1-AR RNA stability indicates that a subject is likely of to have reduced responsiveness to adenosine agonist therapy and also have a small infarct size. In these embodiments, the inventors have discovered two variances in the 3′UTR of A1-AR that act as protective alleles and increase A1-AR RNA stability, where one variance is characterized by an adenosine (A) at position 1689 in SEQ ID NO:1 as compared to a cytosine (C) in the wildtype A1-AR nucleic acid sequence corresponding to SEQ ID NO:1 (e.g. negative control) (this variance is termed “1689C/A” herein, and corresponds to RefSNP identification number rs6427994) and the other variance is characterized by a deletion of a thymine (T) at position 2205 compared to the wildtype control (this variance is termed “2205Tdel” herein, and corresponds to RefSNP identification number rs33912180).

In another embodiment, the presence of particular variances (e.g. change, mutation, polymorphism or SNP) in the human A3-AR gene or gene products relative to a control group (e.g. wildtype, positive and/or negative control group) results in altered A3-AR function. In one embodiment, variances that decrease A3-AR function, and therefore function as “susceptibility alleles” indicate a subject is likely to have a large infarct size and also an increased responsiveness to adenosine receptor agonist therapy. In one embodiment, the susceptibility allele of the A3-AR protein is a Leucine (a change from isoleucine) at amino acid position 284 indicates the subject is likely of having a large infarct size and also an increased responsiveness to adenosine receptor agonist therapy. Thus, in one embodiment, the predisposing allele that contributes to responsiveness to adenosine and/or adenosine agonists and infarct size is a polymorphism or mutation within the coding region of the A3-AR gene. In another embodiment, the predisposing allele that contributes to responsiveness to adenosine and adenosine agonists and infarct size is a polymorphism or mutation is a change of the isoleucine at amino acid position at 284 of SEQ ID NO:3. In some embodiments, the variation changes the isoleucine (Iso) at amino acid position 284 of SEQ ID NO:3 to a Leucine (Leu). In other embodiments, a variance in the coding region of A3-AR is where a cytosine (C) is present at position 1509 in SEQ ID NO:2 as compared to an adenosine (A), which is present in the wildtype nucleic acid sequence for A3-AR which corresponds to SEQ ID NO:3. This variance is termed “1509A/C” or “Iso284Leu” herein, and corresponds to RefSNP identification number rs35511654.

The presence or absence of the polymorphisms and/mutations described above can be determined by any means known in the art. In one embodiment, the methods of the invention encompass the screening for any changes in the nucleic acid sequence of the 3′UTR of the A1-AR gene, and/or the coding region of the A3-AR gene. For example, the nucleotides to be screened include, but are not limited to, the nucleotides located at positions 1689, 2205 and 2683 in the 3′UTR of the A1-AR gene (SEQ ID NO:1) and nucleotides located at positions 1509 in the A3-AR gene (SEQ ID NO:2). Also encompassed within this invention is screening to identify the nucleotides at position −54 and 717 in the A1-AR gene (SEQ ID NO:1). The sequence difference at position 717 of SEQ ID NO:1 corresponds to RefSNP identification number rs10920568.

Also encompassed in the methods of this invention is the screening and/or detection of any change or variation in non-coding region of A1-AR, including 5′ and 3′UTR sequences, and intron sequences of the A1-AR gene, particularly if the variance alters the stability of the A1-AR RNA, and therefore is a predictor of the clinical phenotype in terms of responsiveness to adenosine agonist treatment and predictor infarct size. Changes in non-coding regions also include modifications in the nucleic acid such as methylation and acetylation. In such embodiments, any variances or changes that result in a decreased A1-AR RNA stability or destabilize the A1-AR RNA or function as “susceptibility alleles” are encompassed in this invention and will likely indicate a subject will have a large infarct size and an increased responsiveness to adenosine agonist treatment, whereas variances or changes that result in an increase in A1-AR RNA stability or “protective alleles” are also encompassed in this invention, and identifies subjects likely to have a small infarct size and a decreased or diminished responsiveness to adenosine agonist treatment.

Also encompassed in this invention are methods for screening and/or detection of any variation in the coding region and non-coding region of the A3-AR gene, including 5′ and 3′UTR sequences and intron sequences of A3-AR gene that results in altered function of the A3-AR protein. In particular variances that decrease the function of the A3 adenosine receptor are encompassed in this invention, and therefore is a predictor of the clinical phenotype in terms of responsiveness to adenosine agonist treatment and predictor infarct size. Changes in coding and non-coding regions also include methylation and acetylation. In such embodiments, any variance or change that results in a decreased A3-AR function, and/or decreased stability of A3-AR RNA or destabilizes the A3-AR RNA or other “susceptibility alleles” are encompassed in this invention and will likely indicate a subject will have a large infarct size and an increased responsiveness to adenosine agonist treatment, whereas variances or changes that results in an increase in A3-AR activity or A3-AR RNA stability or other “protective alleles” are also encompassed in this invention, and identifies subjects likely to have a small infarct size and a decreased or diminished responsiveness to adenosine agonist treatment

Alternatively, one can screen for A3-AR for any changes in amino acid sequence. In one embodiment, the invention provide methods to screen for a change at amino acid number 248 of SEQ ID NO:3 (e.g. where there is an leucine compared to a isoleucine in wildtype) which is indicative of a subject likely of having a large infarct size and also an increased responsiveness to adenosine receptor agonist therapy.

In one embodiment, a probe is used to screen for variances (e.g. mutations and/or polymorphisms) in the 3′UTR of the A1-AR gene or the coding region of the A3-AR gene. Variances in the 3′UTR of A1-AR and gene of A2-AR may also be determined via sequence analysis, such as, for example, amplification assays, such as PCR, qPCR, RT-PCR or gene arrays. Alternatively, variances in the human 3′UTR of A1-AR and/or coding region of A3-AR may also be detected in the gene product (e.g. mRNA or protein). Alternatively, probes may also be used for screening for variances in the A3-AR protein.

In one embodiment the biological sample is from a normal subject. In other embodiments, the biological sample is from a subject with coronary artery disease or coronary syndrome. A variance of a ‘susceptibility allele’ in the 3′UTR of the A1-AR gene or coding region of the A3-AR gene is indicative of the presence or of the possibility of future affliction with having a large infarct size on ischemic injury, for example a myocardial infarction. The variance of a ‘susceptibility allele’ in the 3′UTR of A1-AR or coding region of A3-AR is also indicative of a subject being likely to respond to adenosine agonist therapy compared with subjects having the wildtype alleles. For example, susceptibility alleles of this invention include, but are not limited to, nt2683(2777)del36 in the 3′UTR of the A1-AR gene and 1509(1033)A/C Iso284Leu in the A3-AR gene.

Alternatively, some embodiments a variance of a ‘protective allele’ in the 3′UTR of the A1-AR gene or coding region of A3-AR gene is indicative of the presence or the increased risk of a subject having a small infarct size on ischemic injury, for example a myocardial infarction. In some embodiments, a variance of a ‘protective allele’ in the 3′UTR of A1-AR gene or coding region of A3-AR gene also identifies a subject likely to have decreased responsiveness to adenosine agonist therapy as compared with subjects having the nucleic acid for the A1-AR and A3-AR wildtype alleles, i.e. subjects have a sequence difference as compared to SEQ ID NO:1 and SEQ ID NO:2 which correspond to the wild type nucleic acid sequence for A1-AR and A3-AR respectively. For example, protective alleles of this invention include, but are not limited to, nt1689(1278)C/A and the nt2205(1795)Tdel polymorphisms in the 3′UTR of A1-AR gene.

In some embodiments, the detection of the presence or absence of a least one nucleic acid variance can be determined by amplifying a segment of nucleic acid encoding the 3′UTR of the A1-AR gene and/or A3-AR gene. The segment to be amplified can be, for example, 1000 nucleotides in length, 500 nucleotides in length or 100 nucleotides in length or less. The segments to be amplified can include a plurality of variances.

In another embodiment, the stability of the A1-AR RNA, such as A1-AR mRNA or destabilized A1-AR RNA such as A1-AR mRNA can be determined as a predictor of the clinical phenotype in terms of responsiveness to adenosine agonist treatment and predictor infarct size. For instance, a decreased stability of A1-AR RNA indicates a subject is likely to have a large infarct size and also is likely to be responsive to adenosine agonist treatments, whereas increased stability of A1-AR RNA indicates a subject is likely to have a small infarction and is likely to have reduced responsiveness to adenosine treatment.

In another embodiment, the absence or presence of a variance in the human A3-AR gene can be detected by analyzing gene product (e.g. protein). In one embodiment, a probe that specifically binds to a variant of A3-AR gene product such as A3-AR mRNA or A3-AR protein is utilized. In one embodiment, the probe is an antibody that preferentially binds to a variant of A3-AR protein. The presence of a variant of A3-AR predicts the likelihood of a subject having a large infarct size and likely having increased responsiveness to adenosine agonists. Alternatively, the probe can be, for example, an antibody fragment, recombinant fragment, chimeric protein, humanized antibody and an aptamer.

The present invention further provides a novel method for treating subjects affected with or at risk of myocardial infarction. In one embodiment, the subjects are affected with, or at risk of coronary artery disease or coronary syndrome. The methods involve determining whether the 3′UTR of the human A1-AR gene and/or the coding region of the A3-AR gene of the subject contains at least one nucleic acid variance. In some embodiments, where subjects are identified as having a variance at a ‘susceptibility allele’ which results in the destabilization of the A1-AR RNA and/or the A3-AR RNA or alternatively susceptibility allele which results in decreased function of the A1-AR and/or A3-AR proteins, the subject is administered a therapeutically effective amount of a adenosine agonist or adenosine therapy or appropriate treatment for the prevention and/or treatment of infarction. Alternatively, if a subject is identified as having a variance at a ‘protective allele’ which results in an increased stability of the A1-AR RNA, the subjects is likely to have diminished responsiveness to adenosine agonist treatment, and therefore the subject is administered or recommended an alternative therapy to an adenosine agonist for the treatment and/or prevention of infarction.

One aspect of the present invention relates to a method for predicting whether a subject will be responsive to an adenosine agonist treatment, the method comprising: analyzing a sample comprising nucleic acid from a subject for the presence of a sequence difference in the 3′-untranslated region (3′-UTR) of the A1 adenosine receptor gene relative to the 3′-UTR of SEQ ID NO:1, wherein the sequence difference in the 3′-UTR affects the stability of the adenosine receptor A1 RNA as compared with the stability of adenosine receptor A1 RNA corresponding to SEQ ID NO:1, and is a sequence difference is identified that increases the stability of the adenosine receptor A1 RNA relative to the stability of adenosine receptor A1 RNA corresponding to SEQ ID NO:1 it identifies a subject with a likelihood of decreased responsiveness to an adenosine agonist treatment, whereas if a sequence difference is identified that decreases the stability of the adenosine receptor A1 RNA relative to the stability of adenosine receptor A1 RNA corresponding to SEQ ID NO:1 it identifies a subject with a likelihood of increased responsiveness to an adenosine agonist treatment, and if there is no sequence difference identified in the 3′UTR of the A1 adenosine receptor RNA corresponding to SEQ ID NO:1, the subject is identified as being likely to be responsive to an adenosine agonist treatment.

In some embodiments, the sequence difference that identifies a subject with a likelihood of a decreased responsiveness to adenosine agonist treatment is a change of a cytosine (C) in the 3′UTR of the A1 adenosine receptor gene at position 1689 of SEQ ID NO:1 to an adenosine (A), also referred to herein as nt1689(1278)C/A. In some embodiments, the sequence difference that identifies an subject with a likelihood of a decreased responsiveness to adenosine agonist treatment is a deletion of a thymidine (T) in the 3′UTR of the A1 adenosine receptor gene at position 2205 of SEQ ID NO:1, also referred to herein as nt2205(1790)delT. In some embodiments, the sequence difference that identifies an subject with a likelihood of an increased responsiveness to adenosine agonist treatment is a deletion of at least 1 nucleotides in the 3′UTR of the A1 adenosine receptor gene between position 2683 and 2719 of SEQ ID NO:1. In further embodiments, the sequence difference that identifies an subject with a likelihood of an increased responsiveness to adenosine agonist treatment is a deletion of 36 nucleotides in the 3′UTR of the A1 adenosine receptor gene beginning at position 2683 of SEQ ID NO:1, also referred to herein as nt2683(2777)del36.

In some embodiments, an adenosine agonist treatment comprises adenosine, adenosine analogues and adenosine receptor agonists. In some embodiments, a subject has or is at risk of having stable coronary artery disease or acute coronary syndrome.

In some embodiments, the methods as disclosed herein further comprise administering an adenosine agonist treatment to a subject if the subject is identified to have a likelihood of an increased responsiveness to adenosine agonist treatment or identified to be likely to be responsive to an adenosine agonist treatment, for example an adenosine agonist treatment such as, but not limited to, adenosine, adenosine analogues or adenosine receptor agonists.

In alternative embodiments, the methods as disclosed herein, further comprise administering an appropriate non-adenosine agonist treatment or an therapy other than an adenosine agonist to the subject if the subject is identified to have a likelihood of decreased responsiveness to adenosine agonist treatment.

Another aspect of the present invention provides a method for predicting whether a subject will be responsive to an adenosine agonist treatment, the method comprising: analyzing a sample comprising nucleic acid from a subject for the presence of a sequence difference in the nucleic acid sequence encoding the A3 adenosine receptor gene as compared to the nucleic acid sequence corresponding to SEQ ID NO:2, where the sequence difference detected in the nucleic acid sequence affects a function of the A3 adenosine receptor protein as compared with that function of the A3 adenosine receptor protein corresponding to an A3 adenosine receptor having the amino acid sequence of SEQ ID NO:3, and if a sequence difference is detected that decreases the function of the A3 adenosine receptor protein relative to the function of the A3 adenosine receptor protein corresponding to an A3 adenosine receptor having amino acid sequence of SEQ ID NO:3 it identifies a subject with a likelihood of increased responsiveness to an adenosine agonist treatment relative to a subject with A3 adenosine receptor of SEQ ID NO:3, and if there is no sequence difference in the amino acid sequence of the A3 adenosine receptor corresponding to SEQ ID NO:3, the subject is identified as being likely to be responsive to an adenosine agonist treatment.

In some embodiments, the sequence difference is a change in the amino acid coding region of the nucleic acid sequence encoding an A3 adenosine receptor gene corresponding to SEQ ID NO:2, for example the sequence difference results in a change in the amino acid sequence of an A3 adenosine receptor as compared to the amino acid sequence SEQ ID NO:3. In some embodiments, such a sequence difference changes the identity of amino acid number 248 of the human A3 adenosine receptor gene corresponding to SEQ ID NO:3, for example the sequence difference changes an Isoleucine to a Leucine at amino acid 248 of SEQ ID NO:3, also referred to herein as I248L. In some embodiments, sequence difference in SEQ ID NO:3 is an adenosine (A) to a cytosine (C) at the nucleotide corresponding to position 1509 of the nucleic acid corresponding to SEQ ID NO:2 encoding the A3 adenosine receptor gene, also referred to herein as nt1509(1033)A/C.

In some embodiments, the subject has or is at risk of having stable coronary artery disease or acute coronary syndrome.

In some embodiments, the methods as disclosed herein further comprise administering an adenosine agonist treatment to the subject if the subject is identified to have a sequence difference that results in an increased responsiveness to an adenosine agonist treatment, for example an adenosine agonist treatment such as, but not limited to adenosine, adenosine analogues or an adenosine receptor agonists. In some embodiments, an adenosine agonist treatment is for ischemia-reperfusion injury, for example but not limited to myocardial ischemia-reperfusion injury.

Another aspect of the present invention relates to a method for predicting relative infarct size in a subject following ischemia-reperfusion injury, the method comprising: analyzing a sample comprising nucleic acid from a subject for the presence of a sequence difference in the 3′-untranslated region (3′-UTR) of the A1 adenosine receptor gene relative to the 3′-UTR of SEQ ID NO:1, wherein the sequence difference in the 3′-UTR affects the stability of the adenosine receptor A1 RNA as compared with the stability of adenosine receptor A1 RNA corresponding to SEQ ID NO:1, and if a sequence difference is detected that increases the stability of the adenosine receptor A1 RNA relative to the stability of adenosine receptor A1 RNA corresponding to SEQ ID NO:1 identifies a subject with a likelihood of a smaller infarct size relative to a subject with A1 adenosine receptors corresponding to SEQ ID NO:1, and if a sequence difference is detected that decreases the stability of the adenosine receptor A1 RNA relative to the stability of adenosine receptor A1 RNA corresponding to SEQ ID NO:1 identifies a subject with a likelihood of a larger infarct size relative to a subject with A1 adenosine receptors corresponding to SEQ ID NO:1.

In some embodiments, the sequence difference that identifies a subject with a likelihood of a smaller infarct size as compared to a subject with A1 adenosine receptors corresponding to SEQ ID NO:1 is a cytosine (C) to an adenosine (A) change at the nucleotide at position 1689 of SEQ ID NO:1 corresponding to the 3′UTR of the A1 adenosine receptor gene, also referred to herein as nt1689(1278)C/A. In some embodiments, the sequence difference that identifies a subject with a likelihood of a smaller infarct size as compared to a subject with A1 adenosine receptors corresponding to SEQ ID NO:1 is a deletion of a thymidine (T) at position 2205 of SEQ ID NO:1 corresponding to the 3′UTR of the A1 adenosine receptor gene, also referred to herein as nt2205(1790)delT. In some embodiments, the sequence difference that identifies a subject with a likelihood of a larger infarct size as compared to a subject with A1 adenosine receptors corresponding to SEQ ID NO:1 is a deletion of at least 1 nucleotide in the 3′UTR of the A1 adenosine receptor gene beginning at position 2683 of SEQ ID NO:1, also referred to herein as nt2683(2777)del36.

In alternative embodiments, the sequence difference that identifies a subject with a likelihood of a larger infarct size as compared to a subject with A1 adenosine receptors corresponding to SEQ ID NO:1 is a deletion of at least 1 nucleotide in the 3′UTR of the A1 adenosine receptor gene between position 2683 and 2719 of SEQ ID NO:1. In some embodiments, such a sequence difference that identifies a subject with a likelihood of a larger infarct size as compared to a subject with A1 adenosine receptors corresponding to SEQ ID NO:1 is a deletion of 36 nucleotides in the 3′UTR of the A1 adenosine receptor gene beginning at position 2683 of SEQ ID NO:1, also referred to herein as nt2683(2777)del36. It should be noted that in some embodiments, a subject can have at least one sequence difference as disclosed herein, and in some embodiments a subject can have more than one different sequence difference as disclosed herein.

In some embodiments, a subject has or is at risk of having stable coronary artery disease or acute coronary syndrome, and in some embodiments, a subject has ischemia-reperfusion injury, such a, for example but not limited to myocardial infarction. In some embodiments, a subject has, is having, or is at risk of ischemia-reperfusion injury.

In some embodiments, the methods as disclosed herein further comprise administering an adenosine agonist treatment to the subject if the subject is identified to have a likelihood of an increased infarct size, for example, such adenosine agonist treatment include, but are not limited to adenosine analogues and adenosine receptor agonists, or analogues and derivatives thereof.

Another aspect of the present invention relates to a method for predicting relative infarct size in a subject following ischemia-reperfusion injury, the method comprising: analyzing a sample comprising nucleic acid from a subject for the presence of a sequence difference in the nucleic acid sequence encoding the A3 adenosine receptor gene as compared to the nucleic acid sequence corresponding to SEQ ID NO:2, wherein the sequence difference in the nucleic acid sequence affects a function of the A3 adenosine receptor protein as compared with that function of the A3 adenosine receptor protein corresponding to an A3 adenosine receptor having the amino acid sequence of SEQ ID NO:3, wherein if sequence difference is detected that decreases the function of the A3 adenosine receptor protein relative to the function of the A3 adenosine receptor protein corresponding to a receptor having the amino acid sequence of SEQ ID NO:3 identifies a subject with a likelihood of a larger infarct size as compared to a subject with A3 adenosine receptors corresponding to SEQ ID NO:3.

In some embodiments, the sequence difference is a change in the amino acid coding region of the nucleic acid sequence encoding an A3 adenosine receptor gene corresponding to SEQ ID NO:2, for example but not limited to a change in the amino acid sequence of an A3 adenosine receptor as compared to the amino acid sequence SEQ ID NO:3 such as a change of amino acid number 248 of the human A3 adenosine receptor gene corresponding to SEQ ID NO:3. In some embodiments, the sequence difference changes an Isoleucine to a Leucine at amino acid 248 of SEQ ID NO:3, also referred to herein as 1248L.

In some embodiments, the sequence difference in SEQ ID NO:3 is from a change an adenosine (A) to a cytosine (C) at the nucleotide corresponding to position 1509 of the nucleic acid corresponding to SEQ ID NO:2 encoding the A3 adenosine receptor gene, also referred to herein as nt1509(1033)A/C.

In some embodiments, a subject has or is at risk of having stable coronary artery disease or acute coronary syndrome, and in some embodiments a subject has ischemia-reperfusion injury, such a but not limited to myocardial infarction. In some embodiments, a subject has, is having, or is at risk of ischemia-reperfusion injury, and in some embodiments, the myocardial infarction is acute or chronic myocardial infarction.

In some embodiments, the methods as disclosed herein further comprise administering an adenosine agonist to the subject if the subject is identified as having a sequence variation or sequence difference that results in a large infarct size, for example, an adenosine agonist such as, but not limited to adenosine analogues and adenosine receptor agonists.

In some embodiments, administering an adenosine agonist can be prior to onset of ischemia and in some embodiments, administration can be post onset of ischemia, and in some embodiments administration can be substantially concurrent with onset of ischemia.

Another aspect of the present invention relates to methods for directing treatment in a subject, the method comprising testing for a sequence difference in the A1 adenosine receptor 3′UTR as compared to the nucleic acid sequence corresponding to SEQ ID NO:1 in a biological sample obtained from the subject, and/or testing for a sequence difference in the A3 adenosine receptor gene as compared to the nucleic acid corresponding to SEQ ID NO:2 in a biological sample obtained from the subject, wherein if a sequence difference is detected in the 3′UTR of the A1 adenosine receptor gene which corresponds to a deletion of at least one nucleic acid beginning at position 2683 of SEQ ID NO:1, and/or if a sequence difference is detected in the A3 adenosine receptor gene which corresponds to a change in 1509(1033)A/C of SEQ ID NO:2, the subject has an increased likelihood for responsiveness to an adenosine agonist treatment and a clinician directs the subject to be treated with an appropriate adenosine agonist treatment, and wherein if a sequence difference is detected in the 3′UTR of the A1 adenosine receptor gene which corresponds to a change in 1698(1278)C/A of SEQ ID NO:1, and/or corresponds to a change in 2205(1790)Tdel of SEQ ID NO:1, the subject has the likelihood of decreased responsiveness to an adenosine agonist treatment, and a clinician directs the subject to be treated with an appropriate treatment other than an adenosine agonist treatment.

Another aspect of the present invention provides methods for preventing or reducing the risk of a subject having myocardial infarction, the method comprising assessing and predicting the infarct size in the subjects, the method comprising: testing for a sequence difference in the A1 adenosine receptor 3′UTR as compared to the nucleic acid sequence of SEQ ID NO:1 in a biological sample obtained from a subject, and/or testing for a sequence difference in the A3 adenosine receptor gene as compared to the nucleic acid sequence of SEQ ID NO:2 in a biological sample obtained from a subject, wherein a clinician then reviews the results and if a sequence difference is detected in the 3′UTR of the A1 adenosine receptor gene which corresponds to a deletion of at least one nucleic acid beginning at position 2683 of SEQ ID NO:1 and/or if a sequence difference is detected in the A3 adenosine receptor gene which corresponds to a change in 1509(1033)A/C of SEQ ID NO:2, the subject has a likelihood of having a large infarct, and the clinician directs the subject to be treated with an appropriate adenosine agonist treatment.

Another aspect of the present invention relates to kits for detecting the sequence differences in SEQ ID NO:1 and SEQ ID NO:2 as disclosed herein. In some embodiments, the present invention provides kits comprising at least one probe to specifically detect the sequence difference of nt1689(1278)C/A in a nucleotide sequence corresponding to SEQ ID NO: 1. In some embodiments, a kit comprising at least one probe to specifically detect the sequence difference of nt2205(1790)delT in a nucleotide sequence corresponding to SEQ ID NO: 1 is provided. In other embodiments, a kit comprising at least one probe to specifically detect at least one nucleic acid beginning at position 2683 of SEQ ID NO: 1 is provided. In some embodiments, a kit can comprise a probe which can specifically detect the sequence difference of nt2683(2777)del36 in the nucleotide sequence corresponding to SEQ ID NO: 1. In some embodiments, a kit can comprise at least one probe to specifically detect the sequence difference of nt1509(1033)A/C mutation in the nucleotide sequence corresponding to SEQ ID NO: 2.

In some embodiments, a kit can comprise nucleic acid or nucleic acid analogue probes. In some embodiments, a probe can comprise a nucleic acid, nucleic acid analogue, a protein, polypeptide, antibody, antibody fragment, humanized antibody, chimeric antibody, recombinant protein, recombinant antibody, small molecule, aptamer, protein aptamer and variant or fragment thereof. In some embodiments, a probe can be a protein-binding probe. For example, but not limited to, a probe can detect the protein encoded by the nucleic acid sequence SEQ ID NO:3 that has a sequence difference at position 1509 of nt1509(1033)A/C

Another aspect of the present invention provides a kit comprising one or more kits as disclosed herein, and products and reagents, and optionally instructions, to carry out the probe detection of the sequence differences.

In some embodiments, the methods as disclosed herein comprise analyzing a biological sample from the subject for the expression product of an A3 adenosine receptor gene, wherein the detection of the expression product of the A3 adenosine receptor reflects the presence of a sequence difference relative to the A3 adenosine receptor gene corresponding to amino acid sequence SEQ ID NO:3. In some embodiments, the detection comprises the use of an antibody, humanized antibody, recombinant antibody, antibody fragment, aptamer, peptide and analogues.

### BRIEF DESCRIPTION OF DRAWINGS

FIG. 1 shows a comparison of ejection fraction of subjects enrolled in the STICH trial, comparison of lvef versus ef.2d.plax measurements.

FIG. 2 shows a comparison of ejection fraction of subjects enrolled in the STICH trial, comparison of lvef versus lvef2 measurements.

FIG. 3 shows a comparison of ejection fraction of subjects enrolled in the STICH trial, comparison of lvef2 versus ef.2d.plax measurements.

FIG. 4 shows a comparison of left ventricular (LV) end diastolic diameter of subjects enrolled in the STICH trial, comparison of lvedd.vol versus lvd.2D.plax measurements.

FIG. 5 shows a comparison of left ventricular (LV) end systolic diameter of subjects enrolled in the STICH trial, comparison of lvesd.vol versus lvd.2D.plax measurements.

FIG. 6 shows a comparison of infarct size of subjects enrolled in the STICH trial, comparison of lvesd.vol versus lvd.2D.plax measurements.

FIG. 7 shows a baseline characteristics analyzed of subjects enrolled in the STICH trial.

FIG. 8 shows a summary of variables collected from subjects enrolled in the STICH trial

FIG. 9 shows data of all the polymorphisms analyzed in A1-AR, A2A-AR and A3-AR genes with respect to the infarct size and left ventricular end systolic diameter and ejection volume.

FIG. 10 shows the RNA secondary structure of the 1689 C/A sequence variation of SEQ ID NO:1. Panel 10A shows allele C, panel 10B shows allele A in the 3′UTR of the A1-AR gene. The sequence difference site is indicated by a solid arrow. The internal loops of interest are indicated by a two-headed arrow. The hairpin loop is indicated by a vertical dotted arrow. Of note, only the informative part of the secondary structure is shown.

FIG. 11 shows the RNA secondary structure of the 2205 tdel polymorphism. Panel 11A shows the allele TT, Panel 11B shows the allele T in the 3′UTR of the A1-AR gene. The sequence difference site is indicated by a solid arrow. The internal loops of interest are indicated by a two-headed arrow. Of note, only the informative part of the secondary structure is shown.

### DETAILED DESCRIPTION

The present invention is based on the discovery that variances (e.g. changes such as mutations and/or polymorphisms) in the human genes encoding A1 adenosine receptor (A1-AR) and the gene encoding the A3 adenosine receptor (A3-AR) from the wild type sequence are associated with altered risk of having large or small myocardial infarction and also predicts the likelihood of enhanced or diminished response to adenosine receptor agonist therapy.

Accordingly, the present invention provides methods for screening for individuals for mutations and polymorphisms in the human A1 adenosine receptor and the human A3 adenosine receptor. In particular, the invention provides screening of subjects with an increased susceptibility to, or current affliction with a disease or disorder associated with a coronary artery disease or acute coronary syndrome, to identify individuals with the likelihood of an increased risk of having large versus small myocardial infarction and also predicts the likelihood of enhanced or diminished response to adenosine receptor agonist therapy.

Since adenosine receptors appear to play a critically important role in the heart's response to ischemia and to myocardial infarction, the inventors investigated if polymorphisms in the adenosine receptor genes might influence the phenotype of subjects after a myocardial infarction. Indeed, recent studies have demonstrated significant effects of polymorphisms in other trans-membrane G protein-coupled receptors on both the cardiac phenotype and on the heart's response to pharmacologic therapy (McNamara DM, J Am Coll Cardiol. 2004 Nov. 16; 44(10):2019-26; Liggett s. Proc Natl Acad Sci USA. 2006 Jul. 25; 103(30):11288-93. Epub 2006 Jul. 14).

The inventors of the present invention identified polymorphisms in the adenosine receptor genes by sequencing DNA obtained from normal individuals, from patients with ischemic heart disease and left ventricular dysfunction, and from patients with left ventricular function who did not have ischemic heart disease. In addition, the inventors assessed the impact of polymorphisms in each of the adenosine receptor genes on morphology and function in a carefully phenotyped population of patients with coronary artery disease and left ventricular dysfunction

The inventors have discovered that variances (e.g. changes such as mutations and/or polymorphisms) in the human genes encoding A1 adenosine receptor (herein referred to as A1-AR or A1-AR) and the gene encoding the A3 adenosine receptor (herein referred to as A3-AR or A₃-AR) from the wild type sequence are associated with altered risk of having large or small myocardial infarction and also predicts the likelihood of enhanced or diminished response to adenosine receptor agonist therapy.

In particular, the inventors have discovered that variances (mutations and/or polymorphisms) that result in an increased stability of the A1-AR RNA predict or correlate with a small infarct size, whereas variances that destabilize the A1-AR RNA and/or reduce the function and/or decrease the expression of the A3-AR protein predict or correlate with a large infarct size. Further, the inventors have discovered that these variances which increase the stability of the A1-AR RNA identifies subjects unlikely to respond or to have diminished responsiveness to further exogenous adenosine, such as adenosine agonist therapy or ischemia-reperfusion therapy, whereas those variations or sequence differences that result in the destabilization of the A1-AR RNA and/or reduction in the function and/or activity of the A3-AR protein identifies subjects which are likely to respond to exogenous adenosine such as adenosine agonist therapy or in myocardial ischemia-reperfusion therapy.

The inventors have discovered a method to identify subjects with very different responsiveness to adenosine receptor agonists and adenosine therapy based on the variances (mutations and/or polymorphisms) in the non-coding and coding regions of the human A1-AR and human A3-AR genes.

Accordingly, the present invention provides novel methods for screening for individuals for mutations and polymorphisms in the human A1 adenosine receptor and the human A3 adenosine receptor. In particular, the invention provides screening of patients with an increased susceptibility to, or current affliction with a disease or disorder associated with a coronary artery disease or acute coronary syndrome, to identify individuals with the likelihood of an increased risk of having large or small myocardial infarction and also predicts the likelihood of enhanced or diminished response to adenosine receptor agonist therapy.

Mutations and/or Polymorphisms in A1-AR

The methods of this invention disclose polymorphisms or single nucleotide polymorphisms (SNP) and/or mutations in the 3-untranslated region (3′ UTR) of the gene encoding human A1 adenosine receptor (A1-AR) and a mutation in the gene encoding human A3 adenosine receptor (A3-AR) that correlate with altered response to adenosine therapy.

Three polymorphisms in the 3′ UTR of the human A1-AR gene are disclosed that affect secondary structure of RNA: nt1689(1278)C/A; nt2205(1795)Tdel; nt2683(2777)del36. The nucleotide numbers are based on Ensemble cDNA ID: ENSG00000163485 for A1-AR referred to herein as SEQ ID NO:1. The numbers in “( )” are based on the numbering of Deckert et al, (Deckert J., Am J Med gen 81; 18:1988). Since these polymorphisms are not in the coding region of the gene encoding human A1-AR but in the 3′UTR, they do not confer an amino acid change in the polynucleotide sequence. However, they resulted in significant changes in the secondary structure of RNA. The SNP in A1-AR 3′UTR termed nt1689(1278)C/A, is where there is an adenosine (A) at position 1689 in SEQ ID NO:1 as opposed to the wildtype A1-AR nucleic acid sequence corresponding to SEQ ID NO: 1 (e.g. negative control) where a cytosine (C) is present at position 1689, and is alternatively referred to as “C1689A” or RefSNP Identification No: rs6427994. The SNP in the A1-AR 3′UTR termed nt2205(1795)Tdel is where the thymine (T) at position 2205 is absent compared to the wildtype control or wildtype genotype. An individual having a single allele (heterozygous) or two (homozygous) alleles comprising either a A-allele at nt1689(1278)C/A and/or a deletion of the T-allele at the nt2205(1795)Tdel SNPs in the 3′UTR of the human A1-AR gene is associated with an increased likelihood of having a small or decreased infarct size upon myocardial infarction. Furthermore, an individual heterozygous or homozygous for at least one of these two SNPs; nt1689(1278)C/A and/or the nt2205(1795)Tdel is identified as having a likelihood of a diminished responsiveness to adenosine receptor agonists.

In some embodiments, the polymorphisms in A1-AR 3′UTR termed nt2683(2777)del36, is where there is a 36 nucleotide deletion beginning at position 2683 in SEQ ID NO:1 as compared to the wildtype A1-AR nucleic sequence corresponding to SEQ ID NO:1 (e.g. negative controls). Having a single allele (heterozygous) or two alleles (homozygous) for nt2683(2777)del36 in the 3′UTR of the human A1-AR gene identifies subjects with an increased likelihood of having a large infarct size, for example on myocardial infarction. Furthermore, an individual heterozygous or homozygous for the nt2683(2777)del36 variant has or is expected to have an increased responsiveness to adenosine receptor agonists, relative to wild type nucleic acid sequence for A1-AR 3′UTR.

In another embodiment, a polymorphism in the A1-AR gene is in the non-coding 5′UTR, where there is a thymine (T) allele at position −54 in SEQ ID NO:1 as opposed to the wildtype which as a cytosine (C) allele at position −54. This variance is termed −54C/T. In another embodiment, the variance is in the coding region of the human A1-AR gene, where there is a guanine (G) allele at position 717 as opposed to a thymine (T) allele in the wildtype. This variance is termed 717(716)T/G. An individual having a single allele (heterozygous) for −54C/T and/or 717(716)T/G is associated with an increased likelihood of having a large infarct size upon myocardial infarction. Furthermore, an individual having a single allele (heterozygous) of the −54C/T and/or 717(716)T/G polymorphism is associated with an increased responsiveness to adenosine receptor agonists.

Mutations and/or Polymorphisms in A3-AR

In one embodiment, a polymorphism in the coding region of the nucleic acid sequence encoding A3-AR was discovered, 1509(1033)A/C Iso284Leu, where a cytosine (C) is present at position 1509 in SEQ ID NO:2. The nucleotide numbers are based on Ensemble cDNA ID: ENST00000241356 for A3-AR, also referred to as SEQ ID NO:2 herein. In some embodiments, the wildtype A3-AR nucleic sequence corresponding to SEQ ID NO:2 (e.g. negative control), there an adenosine (A) is present at position 1509 which substitutes a Leucine (Leu) for an isoleucine (Iso) at amino acid 248 of the A3-AR amino acid sequence (Accession ID NO: NP_{—}000668, referred to herein as SEQ ID NO:3). The numbers in “( )” are based on the numbering of Deckert et al, (Deckert J., Am J Med gen 81; 18:1988). Individuals with one (heterozygous) or two alleles (homozygous) for “1509(1033)A/C Iso284Leu” are identified to have an increased likelihood of having a large infarct size on myocardial infarction. The presence of such a C-allele in the A3-AR gene in an individual is predictive of increased susceptibility to in a large infarct size and increased responsiveness to adenosine receptor agonists, for example A1-selective agonists, A3-selective agonists, A1/A3 or A2/A3 selective agonists, whereas an A-allele is protective. This polymorphism is sometimes referred to as “A1509C” or “I284L”. Subjects having one (heterozygous) or two (homozygous) C-alleles at position 1509 of SEQ ID NO:2 of the A3-AR gene are predicted to have increased susceptibility of having a large infarct size and increased responsiveness to adenosine receptor agonists.

In another embodiment, the present invention also provides novel methods of screening individuals to determine if they have an increased likelihood to have a diminished or enhanced responsiveness to adenosine receptor agonist treatment. In one embodiment, the presence of the 1509(1033)A/C Iso284Leu on at least one allele of A3-AR gene and/or the nt2683(2777)del36 present on at least one allele of the 3′UTR of the A1-AR gene is predictive of the likelihood of an increased response to adenosine and/or adenosine receptor agonists relative to wild type. In an alternative embodiment, the presence of the SNPs; nt1689(1278)C/A and/or nt2205(1795)Tdel on at least one allele of the 3′UTR of the A1-AR gene is predictive of the likelihood of a subject having a diminished response to adenosine or adenosine receptor agonists. Furthermore, the methods of the present invention may be combined with other diagnostic methods known to those of skill in the art or those discovered subsequently.

In one embodiment, the present invention provides methods of using a probe to screen for variances or differences (e.g. changes, mutations, polymorphisms, SNPs) in either the nucleic acid sequence of the human A1-AR 3′UTR or other non-coding region such as 5′UTR, exons and alternative splice variants of human A1-AR gene. In some embodiments, the present invention provides methods of using a probe to screen for variances or differences (e.g. changes, mutations, polymorphisms, SNPs) the nucleic acid sequence human A3-AR gene or gene products, or differences in the alternative splice variants of A3-AR relative to the wildtype A3-AR nucleic sequence corresponding to SEQ ID NO:2 (e.g. negative controls).

According to the present invention, a “baseline” or “control” or “control group” can include a normal or negative control and/or disease or positive control, against which test samples can be compared. Therefore it can be determined, based on the control, whether the sample to be evaluated for mutations and/or polymorphisms in the human A1-AR 3′UTR and/or A3-AR gene has a measurable difference or substantially no difference, as compared to the control group. In one aspect, the baseline control is a negative control. The negative control has a A1-AR 3′UTR or A3-AR gene as expected in the sample of normal (e.g. healthy, negative control) individual.

As used herein, the term “negative control” typically refers to a population of individuals whose sequence for the A1-AR 3′UTR corresponds to SEQ ID NO:1 or A3-AR corresponds to SEQ ID NO:2. For example, a negative control for A1-AR is a nucleic acid sequence having the wildtype allele for the nucleic acid sequences at 1689, 2205 and 2777 in the A1-AR 3′UTR, for example, the wildtype allele corresponds to the nucleic acid sequence SEQ ID NO:1. In alternative embodiments, negative control with respect to A3-AR is the wild type allele at the nucleic acid sequence 1509 for the A3-AR gene, or the nucleic acid sequence which corresponds to SEQ ID NO:2. As illustrative examples only, there is a C-allele at nucleic acid site 1689, a T-allele at nucleic acid site 2205, or no deletion of the nucleic acid at site 2683 in the 3′UTR of the A1-AR gene (SEQ ID NO:1), or an A-allele at nucleic acid sequence 1509 of the A3-AR gene (SEQ ID NO:2).

In some embodiments of the invention, it may be also be useful to compare the gene expression in a test sample to a baseline that has previously been established from a subject or population having susceptibility to large or small infarct size, or increased or diminished responsiveness to adenosine receptor agonist, in particular the expression of the A3-AR gene expression. Such a baseline level, also referred to herein as a “positive control”, refers to a A1-AR or A3-AR gene expression established from one or preferably a population of individuals who has been diagnosed with or having susceptibility to large or small myocardial infarcts and whom have a similar nucleic acid sequence of the A1-AR 3′UTR or the A3-AR gene.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth.

The term “adenosine receptor” as used herein refers to receptors that mediate signaling of adenosine. For example, such receptors are present on the myocardium (muscle cardiac cells). While activation of the A1 and A3 receptors is cardioprotective, activation of A2A receptor can be deleterious and causes damage to cardiac muscle cells.

The term “nucleic acid” is well known in the art. A “nucleic acid” as used herein will generally refer to a molecule (i.e., strand) of DNA, RNA or a derivative or analog thereof, comprising a nucleobase. A nucleobase includes, for example, a naturally occurring purine or pyrimidine base found in DNA (e.g. an adenine “A,” a guanine “G.” a thymine “T” or a cytosine “C”) or RNA (e.g. an A, a G. an uracil “U” or a C). The term “nucleic acid” encompasses the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

The term “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length.

The terms “polypeptide”, “peptide, eptide”, “gene product”, “expression product” and “protein” are used interchangeably herein to refer to a polymer or oligomer of consecutive amino acid residues.

A “gene” refers to coding sequence of a gene product, as well as non-coding regions of the gene product, including 5′UTR and 3′UTR regions, introns and the promoter of the gene product. In addition to the A1-AR and/or A3-AR gene, other regulatory regions such as the promoter and enhancers for A1-AR and/or A3-AR are contemplated as nucleic acids for use with compositions and methods of the claimed invention. Thus, a nucleic acid may encompass a double-stranded molecule or a double-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “is.” The term “gene” refers to the segment of DNA involved in producing a polypeptide chain, it includes regions preceding and following the coding region as well as intervening sequences (introns) between individual coding segments (exons). A “promoter” is a region of a nucleic acid sequence at which initiation and rate of transcription are controlled. It may contain elements at which regulatory proteins and molecules may bind, such as RNA polymerase and other transcription factors, to initiate the specific transcription of a nucleic acid sequence. The term “enhancer” refers to a cis-acting regulatory sequence involved in the transcriptional activation of a nucleic acid sequence. An enhancer can function in either orientation and may be upstream or downstream of the promoter.

The term “exon” as used herein refers to the normal sense of the term as meaning a segment of nucleic acid molecules, usually DNA, that encodes part of or all of an expressed protein.

The term “non-coding” refers to sequences of nucleic acid molecules that do not encode part or all of an expressed protein. Non-coding sequences include but are not limited to introns, promoter regions, 3′ untranslated regions (3′UTR), and 5′ untranslated regions (5′UTR).

The term “coding region” as used herein, refers to a portion of the nucleic acid, which is transcribed and translated in a sequence-specific manner to produce into a particular polypeptide or protein when placed under the control of appropriate regulatory sequences. The coding region is said to encode such a polypeptide or protein.

With reference to nucleic acids of the invention, the term “isolated nucleic acid” or “isolated polynucleotide” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote. With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

The term “oligonucleotide,” as used herein refers to primers and probes of the present invention, and is defined as a nucleic acid molecule comprised of at least two or more ribo- or deoxyribonucleotides. The exact size of the oligonucleotide will depend on various factors and on the particular application and use of the oligonucleotide. The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and the method used. For example, for diagnostic applications, depending on the complexity of the target sequence, an oligonucleotide probe typically contains 15-25 or more nucleotides, although it may contain fewer nucleotides. The probes as disclosed herein are selected to be substantially complementary to different strands of a particular target nucleic acid sequence. This means that the probes must be sufficiently complementary so as to be able to “specifically hybridize” or anneal with their respective target strands. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarily with the sequence of the target nucleic acid to anneal therewith specifically.

In the context of this invention, the term “probe” refers to a molecule which can detectably distinguish between target molecules differing in structure (e.g. nucleic acid or protein sequence). Detection can be accomplished in a variety of different ways depending on the type of probe used and the type of target molecule. Thus, for example, detection may be based on discrimination on detection of specific binding. Examples of such specific binding include antibody binding and nucleic acid, antibody binding to protein, nucleic acid binding to nucleic acid, or aptamer binding to protein or nucleic acid. Thus, for example, probes can include enzyme substrates, antibodies and antibody fragments, and preferably nucleic acid hybridization probes.

The term “specifically hybridize” refers to the association between two single-stranded nucleic acid molecules of sufficient complementary sequence to permit such hybridization under pre-determined conditions generally used in the art (sometimes the sequences are referred to as “substantially complementary”). In particular, the term specifically hybridize also refers to hybridization of an oligonucleotide with a substantially complementary sequence as compared to non-complementary sequence.

The term “specifically” as used herein with reference to a probe which is used to specifically detect a sequence difference, refers to a probe that identifies a particular sequence difference based on exclusive hybridization to the sequence difference under stringent hybridization conditions and/or on exclusive amplification or replication of the sequence difference.

In its broadest sense, the term “substantially” as used herein in respect to “substantially complementary”, or when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, at least 70%, at least 80% or 85%, at least 90%, at least 93%, at least 95% or 96%, at least 97% or 98%, at least 99% or 100% (the later being equivalent to the term “identical” in this context). For example, identity is assessed over a length of at least 10 nucleotides, or at least 11, 12, 13, 14, 15, 16, 17, 18, 19, 20, 21, 22 or up to 50 nucleotides of the entire length of the nucleic acid sequence to said reference sequence (if not specified otherwise below). Sequence comparisons can be carried out using default GAP analysis with the University of Wisconsin GCG, SEQWEB application of GAP, based on the algorithm of Needleman and Wunsch (Needleman and Wunsch (1970) J. MoI. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions.

In its broadest sense, the term “substantially identical”, when used herein with respect to a nucleotide sequence, means a nucleotide sequence corresponding to a reference or target nucleotide sequence, wherein the percentage of identity between the substantially identical nucleotide sequence and the reference or target nucleotide sequence is at least 60%, at least 70%, at least 80% or 85%, at least 90%, at least 93%, at least 95% or 96%, at least 97% or 98%, at least 99% or 100% (the later being equivalent to the term “identical” in this context). For example, identity is assessed over a length of 10-22 nucleotides, such as at least 10, 11, 12, 13, 14, 15, 16, 17, 18, 19, 20, 21, 22 or up to 50 nucleotides of a nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J. MoI. Biol. 48: 443-453; as defined above). A nucleotide sequence “substantially identical” to a reference nucleotide sequence hybridizes to the exact complementary sequence of the reference nucleotide sequence (i.e. its corresponding strand in a double-stranded molecule) under low stringency conditions, preferably medium stringency conditions, most preferably high stringency conditions (as defined above). Homologues of a specific nucleotide sequence include nucleotide sequences that encode an amino acid sequence that is at least 24% identical, at least 35% identical, at least 50% identical, at least 65% identical to the reference amino acid sequence, as measured using the parameters described above, wherein the amino acid sequence encoded by the homolog has the same biological activity as the protein encoded by the specific nucleotide. The term “substantially non-identical” refers to a nucleotide sequence that does not hybridize to the nucleic acid sequence under stringent conditions. The term “substantially identical”, when used herein with respect to a polypeptide, means a protein corresponding to a reference polypeptide, wherein the polypeptide has substantially the same structure and function as the reference protein, e.g. where only changes in amino acids sequence not affecting the polypeptide function occur. When used for a polypeptide or an amino acid sequence, the percentage of identity between the substantially similar and the reference polypeptide or amino acid sequence is at least 24%, at least 30%, at least 45%, at least 60%, at least 75%, at least 90%, at least 95%, at least 99%, using default GAP analysis parameters as described above. Homologues are amino acid sequences that are at least 24% identical, more preferably at least 35% identical, yet more preferably at least 50% identical, yet more preferably at least 65% identical to the reference polypeptide or amino acid sequence, as measured using the parameters described above, wherein the amino acid sequence encoded by the homolog has the same biological activity as the reference polypeptide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “complementary” as used herein refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is anti-parallel to the first region if the residue is thymine or uracil. Similarly, it is known that a cytosine residue of a first nucleic acid strand is capable of base pairing with a residue of a second nucleic acid strand which is anti-parallel to the first strand if the residue is guanine. A first region of a nucleic acid is complementary to a second region of the same or a different nucleic acid if, when the two regions are arranged in an anti-parallel fashion, at least one nucleotide residue of the first region is capable of base pairing with a residue of the second region. Preferably, the first region comprises a first portion and the second region comprises a second portion, whereby, when the first and second portions are arranged in an anti-parallel fashion, such that at least about 50%, and preferably at least about 75%, at least about 90%, or at least about 95% or at least 100% of the nucleotide residues of the first portion are capable of base pairing with nucleotide residues in the second portion. More preferably, all nucleotide residues of the first portion are capable of base pairing with nucleotide residues in the second portion.

According to the present invention, a “baseline” or “control” or “control group” or “wildtype” are used interchangeably herein, can include a normal or negative control and/or disease or positive control, against which test samples can be compared. Therefore it can be determined, based on the control, whether the sample to be evaluated for mutations and/or polymorphisms in the human A1-AR 3′UTR and/or A3-AR gene has measurable difference or substantially no difference, as compared to the control group.

The terms “variant”, “variance”, “mutation” or “polymorphism” are used interchangeably herein, and refer to a difference in nucleic acid sequence among members if a population of individuals. Polymorphisms can sometimes be referred to as “single nucleotide polymorphism” or “SNP” when they vary at a single nucleotide. In some embodiments, polymorphisms can be synonymous or nonsynonymous. Synonymous polymorphisms when present in the coding region or non-coding region typically do not result in an amino acid change, but can result in altered mRNA stability or altered alternative splice sites. Nonsynonymous polymorphism, when present in the coding region, can result in the alteration of one or more codons resulting in an amino acid replacement in the amino acid chain. Such mutations and polymorphisms may be either heterozygous or homozygous within an individual. Homozygous individuals have identical alleles at one or more corresponding loci on homologous chromosomes, while heterozygous individuals have two different alleles at one or more corresponding loci on homologous chromosomes. A polymorphism is thus said to be “allelic,” in that, due to the existence of the polymorphism, some members of a species carry a gene with one sequence (e.g., the original or wild-type “allele”), whereas other members may have an altered sequence (e.g., the variant or, mutant “allele”). In the simplest case, only one mutated variant of the sequence may exist, and the polymorphism is said to be diallelic. For example, if the two alleles at a locus are indistinguishable in their effects on the organism, then the individual is said to be homozygous at the locus under consideration. If the two alleles at a locus are distinguishable because of their differing effects on the organism, then the individual is said to be heterozygous at the locus. In the present application, typographically, alleles are distinguished “+” and “−”. Using these symbols, homozygous individuals are “+/+”, or “−/−”. Heterozygous individuals are “+/−”. The occurrence of alternative mutations can give rise to triallelic and tetra-allelic polymorphisms, etc. An allele may be referred to by the nucleotide(s) that comprise the mutation. In some instances a “silent mutation” is a synonymous codon change, or silent polymorphism is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation results in a type of non-synonymous codon change, for example a nucleic acid substitution, insertion or deletion resulting in a frameshift, and in some embodiments a stop codon is formed thereby leading to premature termination of a polypeptide chain and a truncated protein. A read-through mutation is another type of non-synonymous codon change that causes the destruction of a stop codon, thereby resulting in an extended polypeptide product. While SNPs can be bi-, tri-, or tetra-allelic, the vast majority of the SNPs are bi-allelic, and are thus often referred to as “bi-allelic markers”, or “di-allelic markers”.

The term a “polymorphic gene” refers to a gene having at least one polymorphic region.

The term “genotype” refers to the specific allelic composition of an entire cell or a certain gene, whereas the term “phenotype” refers to the detectable outward manifestations of a specific genotype.

The term “allele”, as used herein, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation. The term “allelic variant” as used herein refers to a region of the gene of interest having one of a plurality of nucleotide sequences found in that region of the gene in other individuals.

The term “wild-type allele” as used herein refers to an allele of a gene which, when present in two copies in a subject results in a wild-type phenotype. As used herein, the term wild type allele for A1-AR is the nucleic acid sequence corresponding to SEQ ID NO:1. Thus any subject or individual having a different nucleic acid sequence to the nucleic acids corresponding to SEQ ID NO:1 has a different nucleic acids as compared to wild type for the nucleic acid sequence encoding 3′UTR A1-AR. As used herein, the term wild type allele for A3-AR is the nucleic acid sequence corresponding to SEQ ID NO:2. Thus any subject or individual having a different nucleic acid sequence to the nucleic acids corresponding to SEQ ID NO:2 has a different nucleic acids as compared to wild type for the nucleic acid sequence encoding A3-AR.

The term “effective amount” as used herein refers to the amount of therapeutic agent of pharmaceutical composition to alleviate at least some of the symptoms of the disease or disorder.

As used herein, the phrase “Gene expression” is used to refer to the transcription of a gene product into mRNA and is also used to refer to the expression of the protein encoded by the gene.

The terms “coronary artery disease” and “acute coronary syndrome” as used interchangeably herein, and refer to myocardial infarction refer to a cardiovascular condition, disease or disorder, include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, amlady, disorder, sickness, illness, complaint, inderdisposion, affection.

The terms “adenosine therapy” or “adenosine receptor agonists” or simply “adenosine agonists” are used interchangeably herein and refer to use of any treatment that acts as adenosine, adenosine analogues and mimetics and variants thereof, adenosine receptor agonists, selective adenosine agonists and dual activating adenosine agonists and variants and analogues thereof. An adenosine agonist activates at least one adenosine receptor. Alternatively or in addition, expression of adenosine is an adenosine agonist. Adenosine receptor agonists are also intended to refer to treatments that increase endogenous adenosine levels and/or increase the expression of the A1-adenosine receptor and/or A3-AR. Adenosine agonists described herein are known to those of skill in the art.

The term “RNA stability” refers to the stability or in vivo half-life of the RNA of a gene, and includes mRNA. The term “Destabilized RNA” is intended to encompass RNA that has a shortened half life due to specific changes in secondary structure of the RNA, relative to the half-life of wild type RNA. As used herein, stability is “affected” if there is an increased or decreased by a statically significant amount relative to wild-type RNA. Generally, the difference will be, for example, at least 10%, 20%, 30%, 50%, 75% or even 90% or more relative to the wild type half life.

The term “biological sample” as used herein refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g. buccal scrapes), whole blood, plasma, serum, urine, saliva, cell culture, or cerebrospinal fluid. Biological samples also include tissue biopsies, cell culture. The sample can be obtained by removing a sample of cells from a subject, but can also be accomplished by using previously isolated cells (e.g. isolated by another person), or by performing the methods of the invention in vivo. Biological sample also refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. Compositions or methods “comprising” one or more recited elements may include other elements not specifically recited. For example, a composition that comprises an nucleic acid of SEQ ID NO encompasses both the nucleic acid of SEQ ID NO and a larger nucleic acid sequence. By way of further example, a composition that comprises elements A and B also encompasses a composition consisting of A, B and C. The terms “comprising” means “including principally, but not necessary solely”. Furthermore, variation of the word “comprising”, such as “comprise” and “comprises”, have correspondingly varied meanings.

In this specification and the appended claims, the singular forms “a,” “an,” and “the” include plural references unless the context clearly dictates otherwise, and therefore “a” and “an” are used herein to refer to one or to more than one (i.e., at least one) of the grammatical object of the article. By way of example, “an element” means one element or more than one element, and reference to a composition for delivering “an agent” includes reference to one or more agents.

Other than in the operating examples, or where otherwise indicated, all numbers expressing quantities of ingredients or reaction conditions used herein should be understood as modified in all instances by the term “about.”

This invention is further illustrated by the following examples which should not be construed as limiting. The contents of all references cited throughout this application, as well as the figures and tables are incorporated herein by reference. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Susceptibility Alleles and Protective Alleles in Predicting Infarct Size.

In one embodiment of the invention, the screening methods of the present invention predict infarct size, for example after myocardial infarction. In particular the inventors have discovered a method to screen subjects for susceptibility to having a large myocardial infarction in ischemia. In particular the patients have stable coronary artery disease or acute coronary syndrome. In one embodiment, the presence of the 1509(1033)A/C Iso284Leu on at least one allele of A3-AR gene and/or the nt2683(2777)del36 present on at least one allele of the 3′UTR of the A1-AR gene is predictive of an increased infarct size on a future myocardial infarction or occlusion and/or ischemia injury. In an alternative embodiment, the presence of the SNPs nt1689(1278)C/A and/or nt2205(1795)Tdel on at least one allele of the 3′UTR of the A1-AR gene is predictive of a smaller infarct size of future myocardial infarction or occlusion and/or ischemic injury. Furthermore, the methods of the present invention may be combined with other diagnostic methods known to those of skill in the art or those discovered subsequently.

Described herein are changes at specific locations in the nucleic acid sequence in the genes and untranslated regions (UTRs) encoding human A1 adenosine receptor (A1-AR) and/or human A3 adenosine receptor (A3-AR). The changes in the nucleic acid sequence are typically referred to as mutations and/or polymorphisms and sometimes referred to as single nucleotide polymorphisms (SNPs) or polymorphic alleles.

In some embodiments, the present invention also provides methods to predict relative infarct size, in particular a method of assessing the relative susceptibility of a mammal (e.g. a human) to a large or small infarct size upon ischemic injury, for example, predicting the likelihood of subjects at the greatest risk of a large infarction and potentially life threatening heart attack. The method also provides the relative susceptibility of a mammal (e.g. human) to responsiveness to an adenosine agonist treatment. A1-AR and A3-AR genotypes play an important role in identifying subjects likely to respond to an adenosine agonist prior to, during, concurrent with or post myocardial ischemia/reperfusion, for example myocardial infarction ischemia-reperfusion therapy. For example, the presence of mutations and/or polymorphisms of the A1 receptor gene that results in destabilization of the A1-AR RNA or decreased function of the A3-AR protein predicts a large scar, for example but not limited to mutations in 2683(2777)del36, −54C/T and 717(716)T/G in the A1-AR gene, or 1509(1033)A/C in the A3-AR gene, whereas mutations that result in increased stability of the A1-AR RNA predict a small infarct size, for example but not limited to mutations 1689(1278)C/A and 2205(1793)Tdel in the 3′UTR of the human A1-AR gene.

As used herein, the term relative susceptibility of a mammal to infarct size refers to the fact that, among a population of individuals in a population at large, some individuals are more likely to have a large or small infarct size than others. This differential potential is attributable, at least in part to the genetic makeup of the individuals in the population. The term infarct size refers to the region of tissue damage by ischemic injury, and is commonly referred to as the scar or scarring by persons skilled in the art. Measurements of infarct size, for example myocardial infarction, can be done by measuring the percentage of muscle encompassed by the scar, and can be performed by methods commonly used in the art, for example MRI, nuclear imaging and PET scans and imaging techniques, as described in the examples. The size of the infarct can be determined to be small or large in comparison to the population at large. In other words, an infarct that is smaller than that observed in the population at large is termed a small infarct or smaller infarction, whereas an infarct that is larger than that observed in the population at large is termed a large infarct or larger infarction.

In accordance with the present invention, it has been discovered that the presence of a certain polymorphism of the 3′UTR of the A1-AR gene and/or coding region of the A3-AR gene identifies subjects with a greater susceptibility to a large infarct size in humans and an increased responsiveness to adenosine agonist treatment. Thus, the method of the invention for assessing the relative susceptibility of an individual to large infarct size comprises determining whether the individual comprises particular polymorphisms or susceptibility alleles of the A1-AR gene and/or A3-AR gene. In particular, such polymorphisms include polymorphisms 2683(2777)del36 in the 3′UTR of A1-AR and 1509(1033)A/C in the coding region of A3-AR gene.

In an alternative embodiment, it has also been discovered that the presence of a certain polymorphism of the 3′UTR of the A1-AR gene identifies subjects with a greater susceptibility to a smaller infarct size in humans and a reduced responsiveness to adenosine agonist treatment. Thus, the method of the invention for assessing the relative susceptibility of an individual to small infarct size comprises determining whether the individual comprises particular polymorphism or protective 3′UTR of the A1-AR gene. In particular, such polymorphisms include polymorphisms 1689(1287)C/A and 2205(1793)Tdel in the 3′UTR of A1-AR.

In conjunction with the genotyping methods of the present invention, one can also determine the presence of other known risk factors in an individual. For example, risk factors for development of infarct size including extent of coronary artery disease or coronary syndrome, and include but are not limited to cigarette smoking, lack of exercise, hypertension and obesity.

The invention also encompasses predictive medicines, which are based, at least in part, on determination of the identity of a polymorphic region and/or expression level (or a combination of both, of the A1-AR and/or A3-AR gene. For example, information obtained using diagnostic assays described herein is useful for determining if a subject will respond to an agonist treatment. Based on the prognostic information, a clinician can the recommend a regime (for example diet and exercise) or therapeutic protocol useful in limiting the risk of having an infarction, or reduce the risk of having a large infarction in the subject.

In some embodiments, the polymorphisms in the A1-AR and the A3-AR genes disclosed herein are useful for diagnosing, screening for, and evaluating predisposition and prognosis to coronary artery syndrome and coronary disease and related pathologies in humans. The polymorphisms in the A1-AR and the A3-AR genes are also useful in detecting disease or disorder that is already present. A treatment regime can then be implemented, for example, administration of an anti-angiogenic agent. In some embodiments, one begins treatment as soon as possible following detection. This is particularly important in early stages when it may be difficult to diagnose a disease or disorder. Furthermore, such polymorphisms in the A1-AR and the A3-AR genes that result in destabilizing the RNA of A1-AR and/or A3-AR or result in a decreased function of the A1-AR and/or A3-AR protein are useful targets for the development of therapeutic agents.

In some embodiments, the A1-AR and A3-AR are also involved in many other biological processes. Another embodiment of the invention is screening of subjects for increased likelihood of having a disorder or disease that is contributed to in part by a dysfunction in the A1-AR and/or A3-AR. For example but not limited to, the A1-AR inhibits neurons, for example A1-AR inhibits cholinergic neurons in the forebrain cells to induce sleep on extended periods of wakefulness. Therefore, in some embodiments, variants of the A1-AR gene that alter the stability of the A1-AR RNA may identify subjects with an increased likelihood of a sleep disorder. For example, mutations that result in destabilizing the A1-AR RNA may lead to loss of this inhibition of cholinergic neurons and lead to sleep disorders where the subject is awake for prolonged periods of time. Another example is that A1-AR promotes vasoconstriction, therefore, in some embodiments, the variants of the A1-AR gene that alter the stability of the A1-AR RNA identifies subjects with disorders of the circulatory system, for example mutations in the A1-AR resulting in destabilizing of the A1-AR RNA may identify subjects with an increased likelihood of decreased afferent arteriolar pressure. As a further example, A1-AR expressed in preglomerular vessels and tubules regulates renal fluid balance, therefore variants in the A1-AR gene and 3′UTR that results in destabilizing the A1-AR RNA may identify subjects at increased risk of diuresis and natiuresis.

Encompassed within this invention is the screening of subjects for variations or sequence differences and mutations and/polymorphisms of the invention for susceptibility to having or being likely to develop such diseases and disorders, where A1-AR and/or A3-AR contribute to, wholly or in part, to the pathology of the disease. Such diseases are known to persons skilled in the art, and include for example stroke and Parkinson's Disease.

### Screening for Responsiveness to Adenosine Agonists

In some embodiments, the methods of this invention relate to nucleic acid molecules containing polymorphisms, methods and reagents for the detection of the changes in the wildtype sequence of A1-AR 3′UTR and/or A3-AR gene, uses of these polymorphisms for the development of detection reagents, and assays or kits that utilize such reagents. The polymorphisms in A1-AR 3′UTR and A3-AR gene as described herein are useful for diagnosing, screening for, and evaluating predisposition and prognosis of infarct size and related pathologies in humans. Furthermore, these mutations are therefore useful for assessing the likelihood that a patient with acute of coronary syndrome or stable coronary artery disease will have small or large infarct. Therefore an appropriate treatment regime can be implicated, for example administration of a prophylactic therapy to reduce the chance of myocardial ischemia and/or change of lifestyle/exercise routine. Preferably, one begins treatment as soon as possible. This is particularly important in early stages when it may be difficult to predict a subject's pathological response to myocardial ischemia, and especially when a patient is at risk of myocardial ischemia when a subject is at risk of developing coronary arterial disease or coronary syndrome.

In another important embodiment, the present invention also provides novel methods of screening individuals to determine if they have an increased likelihood to have a diminished or enhanced responsiveness to adenosine receptor agonist treatment. In one embodiment, the presence of the C-allele at position 1509(1033)A/C Iso284Leu on at least one allele of A3-AR gene and/or the deletion of 36 nucleotides at 2683(2777)del36 on at least one allele of the 3′UTR of the A1-AR gene is predictive of the likelihood of a increased response to adenosine and/or adenosine receptor agonists.

In one important embodiment of the invention, the methods of the invention describe a screening method for determining a subject's responsiveness to adenosine agonist treatments. Accordingly, the method of this invention also encompass that if a subject is identified as to be likely to be responsive to an adenosine agonist treatment, for example subjects with the 36 nucleotide deletion in the A1-AR gene and/or the C-allele at position 1509 in the A3-AR gene they are administered an effective amount of adenosine agonist and/or adenosine receptor agonist. In some embodiments, the treatment is any means to activate the adenosine pathway and/or adenosine receptors. In some embodiments, the treatment is an adenosine or adenosine analogue, for example orally available adenosine analogues. In other embodiments, the treatment is administering an adenosine receptor agonist. For example, the adenosine receptor agonist may be an A1-AR selective agonist, for example 2-chloro-N6-CyClopentyladenosine (CCPA), N6-cyclohexyladenosine (CHA) and adenosine amine congener (ADAC). In other embodiments, the adenosine receptor agonist may be an A3-AR selective agonist, for example N6-(3-isolbenzyl)adenosine-51-N-methyluronamide (IB-MECA), and CI_IB_MECA, MRS584, MRS537, MRS1340 and DBXMA. In an alternative embodiment, the adenosine receptor agonist may be a compound that activates the A1 and A3 receptors simultaneously, for example MRS646 and MRS1364 (see U.S. Pat. No. 9,850,047). Alternatively, adenosine agonists that are A1-, A2- and/or A3-receptor agonists are encompassed for use in the invention, as well as any adenosine agonists that simultaneously activate any combination or all of the A1, A2 and A3 adenosine receptors, for example the A1/A2 adenosine receptor agonist, such as AMP579 (see Patent Application 2004020248928, which is specifically incorporated herein by reference.

In other embodiments, an adenosine agonist or pharmaceutically acceptable derivative is selected from the group including: but not limited to AB-MECA V6-4-amino benzyl-5′-N-methylcarboxamidoadenosine), CPA (N6-cyclopentyladenosine), ADAC (N6-[4-[[[4-[[[(2-aminoethyl)amino]carbonyl]methyl]-anilino]carbonyl]methyl]phenyl]adenosine), CCPA (2-chloro-N6-cyclopentyladenosine), CHA (N6-cyclohexyladenosine), GR79236 (1V6-[1S,trans,2-hydroxycyclopentyl]adenosine), S-ENBA ((2S)—N6-(2-endonorbanyl)adenosine), IAB-MECA (1V6-(4-amino-3-iodobenzyl)adenosine-5′-N-methylcarboxamidoadenosine), R-PIA (R—N6-(phenylisopropyl) adenosine), ATL146e (4-[3-[6-amino-9-(5-ethylcarbamoyl-3,4 dihydroxy-tetrahydro-furan-2-yl)-9H-purin-2-yl]-prop-2-ynyl]-cyclohexanecarboxylic acid methyl ester), CGS-21680 (APEC or 2-Lp-(2-carbonyl-ethyl)-phenyl ethyl amino]-5′-N-ethylcarboxamidoadenosine), CV1808 (2-phenylaminoadenosine), HENECA (2-hex-1-ynyl-5′-N-ethylcarboxamidoadenosine), NECA (5′-N-ethyl-carboxamido adenosine), PAPA-APEC (2-(4-[2-[(4-aminophenyl)methyl carbonyl]ethyl]phenyl)ethylamino-5′-N-ethyl carboxamidoadenosine), DITC APEC(2-[p-(4-isothiocyanatophenyl aminothiocarbonyl-2-ethyl)-phenylethylamino]-15′-N-ethylcarboxamidoadenosine), DPMA (N6-(2(3,5-dimethoxy phenyl)-2-(2-methyl phenyl)ethyl)adenosine), S-PHPNECA ((S)-2-phenylhydroxypropynyl-5′-N ethylcarbox amidoadenosine), WRC-0470 (2 cyclohexylmethylidenehydrazinoadenosine), AMP-579 (1S-[1a,2b,3b,4a(S*)]]-4-[7[[2-(3-chloro-2-thienyl)-1-methylpropyl]amino]-3H-imidazo[4,5-b]pyridyl-3-yl]cyclopentane carboxamide), IB-MECA (N6-(3-iodobenzyl) adenosine −5′-N methyluronamide), 2-CIADO (2-chloroadenosine), I-ABA (N6-(4-amino-3-1 iodobenzyl)adenosine), S-PIA (S—N6-(phenylisopropyl)adenosine), 2-[(2-aminoethyl aminocarbonylethyl)phenylethyl amino]-5′-N-ethyl-carboxamidoadenosine, 2-C1-IB MECA (2-chloro-Ni-(3-iodobenzyl)adenosine-5′-N-methyluronamide), polyadenylic acid, and any mixture thereof. Thus these compound represent functional mimetics and variants of adenosine as the terms are used herein.

In alternative embodiments, the subject is administered a treatment or therapeutic compound that functions through the activation of adenosine pathway, and includes compounds already known by persons skilled in the art and compounds that have yet to be developed.

In another embodiment, the Polymorphisms in A1-AR 3′UTR and A3-AR gene as described herein are also useful for screening for and evaluating the likelihood of a subject having diminished responsiveness to adenosine receptor agonist therapy and/or treatment. In such an embodiment, the presence of the Polymorphisms; nt1689(1278)C/A and/or nt2205(1795)Tdel on at least one allele of the 3′UTR of the A1-AR gene is predictive of the likelihood of a subject having a diminished response to adenosine or adenosine receptor agonists. Furthermore, the methods of the present invention may be combined with other diagnostic methods known to those of skill in the art or those discovered subsequently. Accordingly, if a subject is identified to have a diminished responsiveness to adenosine, adenosine agonists and/or adenosine receptor agonists, a more suitable treatment regime other than treatments and therapies that function through the adenosine pathway can be implicated. For example, the appropriate treatment regime may be a treatment or therapeutic that functions through the adenosine pathway, or other treatments for infarction can be implicated. In some embodiments, the methods describe methods for analysis of mutations and/or polymorphisms in the 3′UTR of the human A1-AR gene. In some embodiments, and A3-AR gene as described herein are also useful for screening for and evaluating the likelihood of responsiveness to adenosine receptor agonist therapy and/or treatment.

In some embodiments, one begins treatment as soon as possible, and in some embodiments the effective dose is adjusted accordingly depending on the responsiveness to the adenosine receptor agonists. This embodiment is particularly important in screening populations of subjects for the effectiveness of an adenosine therapy, for example an adenosine receptor agonist for a particular pathology. For example, an adenosine agonist may be identified not to have a therapeutic effect if some subjects tested with the adenosine agonist have diminished responsiveness to adenosine agonists, for example if the subjects have at least one allele for 1689(1278)C/A and/or at least one allele for 2205(1795)Tdel of the 3′UTR of the A1-AR gene, and therefore are genetically predisposed to have a diminished responsiveness to adenosine agonists and/or adenosine therapy.

It is understood that screening is used for screening of responsiveness to any therapy that functions through adenosine or the adenosine pathway and/or A1-AR and/or A3-AR receptors. The methods of the invention are not necessarily limited to the responsiveness to adenosine-agonists only.

Clinical trials have shown that patient response to treatment with pharmaceuticals is often heterogeneous. There is a continuing need to improve pharmaceutical agent design and therapy. In that regard, polymorphisms as disclosed herein or other sequence variations or sequence differences can be used to identify patients most suited to therapy with particular pharmaceutical agents (this is often termed “pharmacogenomics”). Similarly, polymorphisms or other sequence variations or sequence differences can be used to exclude patients from certain treatment due to the patient's increased likelihood of developing toxic side effects or their likelihood of not responding to the treatment. Pharmacogenomics can also be used in pharmaceutical research to assist the drug development and selection process. (Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 3).

The ability to target populations expected to show the highest clinical benefit, based on the normal or disease genetic profile, can permit: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

Accordingly, the methods of the invention can be used to assess and/or re-assess ther therapeutic effectiveness of existing and novel therapeutic compounds and drugs, for example therapeutic adenosine agonists that are currently on the market, or alternatively those that failed to make it to market. For example, the methods of the invention can be used to re-assess current adenosine treatments, for example adenosine, adenosine-agonists and analogues or variants thereof, for example, agonists selective for A1-, A2- and/or A3-receptors, as well as any adenosine agonist that simultaneously activates any combination or all of the A1, A2 and A3 adenosine receptors, that have not been proven successful as adenosine agonists in subjects, for example subjects with acute coronary syndrome, for example myocardial infarction. For example, one such compound is the A1/A2 adenosine receptor agonist AMP579 (1S-[1a,2b,3b,4a(S*)]]-4-[7[[2-(3-chloro-2-thienyl)-1-methylpropyl]amino]-3H-imidazo[4,5-b]pyridyl-3-yl]cyclopentane carboxamide) (see U.S. Patent Application 2004020248928 which is incorporated herein by reference). Examples of other adenosine agonists are described in PCT application 05003150, PCT 9850047, U.S. Patent Application 2004020248928, and can be selected from a group comprising, but are not limited to, A1-AR selective agonists CCPA, CHA, ADAC, CI-IB-MECA, MRS584, MRS537, MRS1340 and DBXMA, MRS646, MRS1364 (see U.S. Pat. No. 9,850,047), AB-MECA, CPA, ADAC, GR79236, S— ENBA, IAB-MECA, R-PIA, ATL146e, CGS-21680, CV1808, HENECA, NECA, PAPA-APEC, DITC APEC DPMA, S—PHPNECA, WRC-0470, AMP-579, IB-MECA, 2-CIADO, I-ABA, S-PIA, 2-[(2-aminoethyl aminocarbonylethyl)phenylethyl amino]-5′-N-ethyl-carboxamidoadenosine, 2-C1-IB MECA, polyadenylic acid, and any mixture thereof.

In one embodiment, the methods encompass screening for the polymorphisms and variants in the A1-AR and/or the A3-AR gene, in particular the mutations and polymorphisms of the present invention in candidates enrolled in past, present and future clinical studies of adenosine agonist and adenosine receptor agonists, for example A1-AR selective agonists, A3-AR selective agonists, A1/A3-AR dual or bifunctional agonists, oral adenosine analogues or modifications or variations or sequence differences thereof, to evaluate their effectiveness, taking into account the genotype of the subjects enrolled in the study with respect to their responsiveness to the adenosine agonist treatment.

### Detection of Mutations or Polymorphisms

According to the present invention, any approach that detects mutations or polymorphisms in a gene can be used, including but not limited to single-strand conformational polymorphism (SSCP) analysis (Orita et al. (1989) Proc. Natl. Acad. Sci. USA 86:2766-2770), heteroduplex analysis (Prior et al. (1995) Hum. Mutat. 5:263-268), oligonucleotide ligation (Nickerson et al. (1990) Proc. Natl. Acad. Sci. USA 87:8923-8927) and hybridization assays (Conner et al. (1983) Proc. Natl. Acad. Sci. USA 80:278-282). Traditional Taq polymerase PCR-based strategies, such as PCR-RFLP, allele-specific amplification (ASA) (Ruano and Kidd (1989) Nucleic Acids Res. 17:8392), single-molecule dilution (SMD) (Ruano et al. (1990) Proc. Natl. Acad. Sci. USA 87:6296-6300), and coupled amplification and sequencing (CAS) (Ruano and Kidd (1991) Nucleic Acids Res. 19:6877-6882), are easily performed and highly sensitive methods to determine haplotypes (Michalatos-Beloin et al. (1996) Nucleic Acids Res. 24:4841-4843; Barnes (1994) Proc. Natl. Acad. Sci. USA 91:5695-5699; Ruano and Kidd (1991) Nucleic Acids Res. 19:6877-6882).

In one embodiment, a long-range PCR (LR-PCR) is used to detect mutations or polymorphisms. LR-PCR products are genotyped for mutations or polymorphisms using any genotyping methods known to one skilled in the art, and haplotypes inferred using mathematical approaches (e.g., Clark's algorithm (Clark (1990) Mol. Biol. Evol. 7:111-122).

For example, methods including complementary DNA (cDNA) arrays (Shalon et al., Genome Research 6(7):639-45, 1996; Bernard et al., Nucleic Acids Research 24(8):1435-42, 1996), solid-phase mini-sequencing technique (U.S. Pat. No. 6,013,431, Suomalainen et al. Mol. Biotechnol. Jun; 15(2):123-31, 2000), ion-pair high-performance liquid chromatography (Doris et al. J. Chromatogr. A May 8; 806(1):47-60, 1998), and 5′ nuclease assay or real-time RT-PCR (Holland et al. Proc Natl Acad Sci USA 88: 7276-7280, 1991), or primer extension methods described in the U.S. Pat. No. 6,355,433, can be used.

In one embodiment, the primer extension reaction and analysis is performed using PYROSEQUENCING™ (Uppsala, Sweden) which essentially is sequencing by synthesis. A sequencing primer, designed directly next to the nucleic acid differing between the disease-causing mutation and the normal allele or the different SNP alleles is first hybridized to a single stranded, PCR amplified DNA template from the individual, and incubated with the enzymes, DNA polymerase, ATP sulfurylase, luciferase and apyrase, and the substrates, adenosine 5′ phosphosulfate (APS) and luciferin. One of four deoxynucleotide triphosphates (dNTP), for example, corresponding to the nucleotide present in the mutation or polymorphism, is then added to the reaction. DNA polymerase catalyzes the incorporation of the dNTP into the standard DNA strand. Each incorporation event is accompanied by release of pyrophosphate (PPi) in a quantity equimolar to the amount of incorporated nucleotide. Consequently, ATP sulfurylase converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a PYROGRAM™. Each light signal is proportional to the number of nucleotides incorporated and allows a clear determination of the presence or absence of, for example, the mutation or polymorphism. Thereafter, apyrase, a nucleotide degrading enzyme, continuously degrades unincorporated dNTPs and excess ATP. When degradation is complete, another dNTP is added which corresponds to the dNTP present in for example the selected SNP. Addition of dNTPs is performed one at a time. Deoxyadenosine alfa-thio triphosphate (dATPS) is used as a substitute for the natural deoxyadenosine triphosphate (dATP) since it is efficiently used by the DNA polymerase, but not recognized by the luciferase. For detailed information about reaction conditions for the PYROSEQUENCING™, see, e.g. U.S. Pat. No. 6,210,891, which is herein incorporated by reference in its entirety.

Another example of the methods useful for detecting mutations or polymorphisms is real time PCR. Real-time PCR systems rely upon the detection and quantification of a fluorescent reporter, the signal of which increases in direct proportion to the amount of PCR product in a reaction. Examples of real-time PCR method useful according to the present invention include, TAQMAN® and molecular beacons, both of which use hybridization probes relying on fluorescence resonance energy transfer (FRET) for quantitation. TAQMAN® Probes are oligonucleotides that contain a fluorescent dye, typically on the 5′ base, and a quenching dye, typically located on the 3′ base. When irradiated, the excited fluorescent dye transfers energy to the nearby quenching dye molecule rather than fluorescing, resulting in a nonfluorescent substrate. TAQMAN® probes are designed to hybridize to an internal region of a PCR product (ABI 7700 (TAQMAN®), Applied BioSystems, Foster City, Calif.). Accordingly, two different primers, one hybridizing to the mutation or polymorphism and the other to the corresponding wildtype allele, are designed. The primers are consequently allowed to hybridize to the corresponding nucleic acids in the real time PCR reaction. During PCR, when the polymerase replicates a template on which a TAQMAN® probe is bound, the 5′ exonuclease activity of the polymerase cleaves the probe. Consequently, this separates the fluorescent and quenching dyes and FRET no longer occurs. Fluorescence increases in each cycle, proportional to the rate of probe cleavage.

Molecular beacons also contain fluorescent and quenching dyes, but FRET only occurs when the quenching dye is directly adjacent to the fluorescent dye. Molecular beacons are designed to adopt a hairpin structure while free in solution, bringing the fluorescent dye and quencher in close proximity. Therefore, for example, two different molecular beacons are designed, one recognizing the mutation or polymorphism and the other the corresponding wildtype allele. When the molecular beacons hybridize to the nucleic acids, the fluorescent dye and quencher are separated, FRET does not occur, and the fluorescent dye emits light upon irradiation. Unlike TAQMAN® probes, molecular beacons are designed to remain intact during the amplification reaction, and must rebind to target in every cycle for signal measurement. TAQMAN® probes and molecular beacons allow multiple DNA species to be measured in the same sample (multiplex PCR), since fluorescent dyes with different emission spectra may be attached to the different probes, e.g. different dyes are used in making the probes for different disease-causing and SNP alleles. Multiplex PCR also allows internal controls to be co-amplified and permits allele discrimination in single-tube assays. (Ambion Inc, Austin, Tex., TechNotes 8(1)—February 2001, Real-time PCR goes prime time).

Yet another method useful according to the present invention for detecting a mutation or polymorphism is solid-phase mini-sequencing (Hultman, et al., 1988, Nucl. Acid. Res., 17, 4937-4946; Syvanen et al., 1990, Genomics, 8, 684-692). In the original reports, the incorporation of a radiolabeled nucleotide was measured and used for analysis of the three-allelic polymorphism of the human apolipoprotein E gene. The method of detection of the variable nucleotide(s) is based on primer extension and incorporation of detectable nucleoside triphosphates in the detection step. By selecting the detection step primers from the region immediately adjacent to the variable nucleotide, this variation can be detected after incorporation of as few as one nucleoside triphosphate. Labelled nucleoside triphosphates matching the variable nucleotide are added and the incorporation of a label into the detection step primer is measured. The detection step primer is annealed to the copies of the target nucleic acid and a solution containing one or more nucleoside triphosphates including at least one labeled or modified nucleoside triphosphate, is added together with a polymerizing agent in conditions favoring primer extension. Either labeled deoxyribonucleoside triphosphates (dNTPs) or chain terminating dideoxyribonucleoside triphosphates (ddNTPs) can be used. The solid-phase mini-sequencing method is described in detail, for example, in the U.S. Pat. No. 6,013,431 and in Wartiovaara and Syvanen, Quantitative analysis of human DNA sequences by PCR and solid-phase minisequencing. Mol Biotechnol 2000 June; 15(2):123-131.

Another method to detect mutations or polymorphisms is by using fluorescence tagged dNTP/ddNTPs. In addition to use of the fluorescent label in the solid phase mini-sequencing method, a standard nucleic acid sequencing gel can be used to detect the fluorescent label incorporated into the PCR amplification product. A sequencing primer is designed to anneal next to the base differentiating the disease-causing and normal allele or the selected SNP alleles. A primer extension reaction is performed using chain terminating dideoxyribonucleoside triphosphates (ddNTPs) labeled with a fluorescent dye, one label attached to the ddNTP to be added to the standard nucleic acid and another to the ddNTP to be added to the target nucleic acid.

Alternatively, an INVADER® assay can be used (Third Wave Technologies, Inc (Madison, Wis.)). This assay is generally based upon a structure-specific nuclease activity of a variety of enzymes, which are used to cleave a target-dependent cleavage structure, thereby indicating the presence of specific nucleic acid sequences or specific variations thereof in a sample (see, e.g. U.S. Pat. No. 6,458,535). For example, an INVADER® operating system (OS), provides a method for detecting and quantifying DNA and RNA. The INVADER® OS is based on a “perfect match” enzyme-substrate reaction. The INVADER® OS uses proprietary CLEAVASE® enzymes (Third Wave Technologies, Inc (Madison, Wis.)), which recognize and cut only the specific structure formed during the INVADER® process which structure differs between the different alleles selected for detection, i.e. the disease-causing allele and the normal allele as well as between the different selected SNPs. Unlike the PCR-based methods, the INVADER® OS relies on linear amplification of the signal generated by the INVADER® process, rather than on exponential amplification of the target.

In the INVADER® process, two short DNA probes hybridize to the target to form a structure recognized by the CLEAVASE® enzyme. The enzyme then cuts one of the probes to release a short DNA “flap.” Each released flap binds to a fluorescently-labeled probe and forms another cleavage structure. When the CLEAVASE® enzyme cuts the labeled probe, the probe emits a detectable fluorescence signal.

Mutations or polymophisms may also be detected using allele-specific hybridization followed by a MALDI-TOF-MS detection of the different hybridization products. In the preferred embodiment, the detection of the enhanced or amplified nucleic acids representing the different alleles is performed using matrix-assisted laser desorption ionization/time-of-flight (MALDI-TOF) mass spectrometric (MS) analysis. This method differentiates the alleles based on their different mass and can be applied to analyze the products from the various above-described primer-extension methods or the INVADER® process.

In one embodiment, a haplotyping method useful according to the present invention is a physical separation of alleles by cloning, followed by sequencing. Other methods of haplotyping, useful according to the present invention include, but are not limited to monoallelic mutation analysis (MAMA) (Papadopoulos et al. (1995) Nature Genet. 11:99-102) and carbon nanotube probes (Woolley et al. (2000) Nature Biotech. 18:760-763). U.S. Patent Application No. US 2002/0081598 also discloses a useful haplotyping method which involves the use of PCR amplification.

Computational algorithms such as expectation-maximization (EM), subtraction and PHASE are useful methods for statistical estimation of haplotypes (see, e.g., Clark, A. G. Inference of haplotypes from PCR-amplified samples of diploid populations. Mol Biol Evol 7, 111-22. (1990); Stephens, M., Smith, N. J. & Donnelly, P. A new statistical method for haplotype reconstruction from population data. Am J Hum Genet 68, 978-89. (2001); Templeton, A. R., Sing, C. F., Kessling, A. & Humphries, S. A cladistic analysis of phenotype associations with haplotypes inferred from restriction endonuclease mapping. II. The analysis of natural populations. Genetics 120, 1145-54. (1988)).

### Detection of Mutant A3-AR Protein

In one embodiment, one can also look for such changes in the corresponding A3-AR gene product (SEQ ID NO:3). This can be readily done by standard means such as antibodies that recognize specific epitopes. In one embodiment, one can generate antibodies that will only recognize specific amino acid sequences. For example, antibodies that recognize the polymorphic protein (or protein fragment) of SEQ ID NO: 3 and do not recognize the wildtype protein (or protein fragment) are encompassed. In one embodiment one can use an antibody to recognize different types of mutations, for example mutations that result in truncations of the protein or changes in its level of expression. Antibodies and antibody fragments, polyclonal or monoclonal, can be purchased from a variety of commercial suppliers, or may be manufactured using well-known methods, e.g., as described in Harlow et al., Antibodies: A Laboratory Manual, 2nd Ed; Cold Spring Harbor Laboratory Press, Cold Spring Harbor, N.Y. (1988).

The term “antibody” is meant to be an immunoglobulin protein that is capable of binding an antigen. Antibody as used herein is meant to include antibody fragments, e.g. F(ab′)2, Fab′, Fab, capable of binding the antigen or antigenic fragment of interest. Preferably, the antibody is diagnostic in that it discriminatively binds to either the wildtype or the A3-AR predisposing allele of the A3-AR protein described herein.

### Detection of Destabilized A1-AR RNA

The analysis of the stability of the A1-AR RNA and/or A3-AR RNA can be assessed by method known by persons skilled in the art, for example assessed using the mfold program described by Zuker using default parameters (Zuker et al, 2003; Nucleic acid res; 31; 3406-3415) and alternative methods described by Chen (Chen et al, Hum Genet, 2006; 120; 301-333), the references incorporated herein in their entirety by reference.

One can use any method commonly known by persons of ordinary skill in the art to assess the stability of mRNA. For example, one can predict RNA secondary structure based on secondary structure. For example, as disclosed in Hofacker et al. (1995) and Gruner et al. (1995). The number of possible secondary structures (S) of n bases with k base pairs is given as

$$S ( n , k ) = \frac{1}{k} ( \begin{matrix}n - k \\ k + 1\end{matrix} ) ( \begin{matrix}n - k + 1 \\ k - 1\end{matrix} )$$

A number of strategies for predicting secondary structure have been developed. Gruner et al. provide a taxonomy of folding algorithms, and references for each algorithm, for example, Minimum free energy, kinetic folding, 5′-3′ folding, partition function, stochastic factors such as simulated annealing and pseudo-knots.

In some embodiments, the RNA secondary structure can be predicted with some accuracy by computer (for example, the RNAsoft web server; http://www.masoft.ca/), and many bioinformatics applications use some notion of secondary structure in analysis of RNA, for example more general methods are based on stochastic context-free grammars. A web server that implements a type of dynamic programming is Mfold (http://bioweb.pasteur.fr/seqanal/interfaces/mfold-simple.html). One can use any method commonly known by persons of ordinary skill in the art to assess RNA stability in the methods as disclosed herein, for example such methods are disclosed in Hu et al, Nucleic Acids Res, 2003, 31; 3446-3449, Chrzanowska-Lightowles et al, RNA, 2001; 7; 435-444; J. Ross, Microbiol. Rev., 1995, 59:423-450; Pavliceka et al., Trends in Genetics, 2006, 22: 69-73; Akgüla, et al, Archives of Biochem and Biophy, 2007; 459; 143-150; Fritz et al. “An in Vitro Assay to Study Regulated mRNA Stability” Science's STKE, December 2000, which are incorporated herein by reference.

There are several RNA stability elements known to affect RNA stability. These include IRE with bound IRE-BP appears to mask a site that is destabilizing element. This site when masked by IRE-BP prevents degradation, for example when a 50 kd protein binds to histone 3′ UTR in region (6 bp stem/4 base loop) that is required for cell-cycle regulation of stability. Another RNA stability element is a ribonucleotide reductase subunits (RR1 and RR2), which allows the binding of two proteins bind to 3′ UTR=57 (RR1) and 45 (RR2) kd proteins, and a second protein of 75 kd is known to bind to another 3′ UTR region in RR2 and functions as a stabilizer. There is a good correlation between changes in level/binding activity of these protein to the 3′ UTR and mRNA stability in vivo and in vitro.

Other RNA stability elements in the 3′UTR include, for example ARE (AUUA) or AU-rich elements. For example, ARE (AUUUA)=AU-Rich element confers instability on a number of otherwise stable mRNAs (see Lodish FIG. 12-43 and Chen and Shyu Table II). Elements may be complex and composed of several types including: 1. AUUUA with coupled nearby U-rich region or stretch, 2. At least two overlapping nonamers in a U-rich region [UUAUUUA(U/A)(U/A)], 3. U-rich regions.

In some embodiments, the RNA instability elements aren't all equivalent and may lead to different affects on different mRNAs and in different cells. There may be more than one element in different locations within the mRNA, and their effect may vary/cell types and during changes in cellular metabolism. Without being bound by theory and as an non-limiting example, elements from c-fos vs. c-myc may have different affects on different mRNAs in different cells. ARE binding proteins have been identified that are nuclear or cytoplasmic and may shuttle between two. There is a good correlation between ARE-binding protein abundance or activity and increase or decrease in mRNA decay rates. ARE-binding proteins affect mRNA stability in cell-free extracts

Some common RNA stability elements identified in the 3′URT include, for example, AUAGAU and GAU motifs.

Detection of Novel Polymorphisms in Non-Coding Regions of A1-AR and/or Coding and Non-Coding Regions of A3-AR.

Allelic variation associated with infarct size and responsiveness to adenosine agonists may be located within a coding region of A1-AR and/or A3-AR genes or a non-coding regions of the A1-AR and/or A3-AR genes. Non-coding regions include, for example, intron sequences as well as 5′ and 3′ untranslated sequences. In one embodiment, the alleles that are associated with infarct size and responsiveness to adenosine agonists are located within a 3′UTR portion of the A1-AR gene, and in some embodiments they are located in the coding region of the A3-AR gene. Changes of interest in a non-coding region include modifications of the nucleic acid such as methylation.

Another embodiment of the invention provides methods for identifying novel polymorphisms in the human A1-AR and/or A3-AR gene which are associated with infarct size and responsiveness to adenosine agonists. The strength of the association between a polymorphic allele and infarct size and/or responsiveness to adenosine agonists can be characterized by a particular odds ratio such as an odds ratio of at least 2 with a lower 95% confidence interval limit of greater than 1. Such an odds ratio can be, for example, at least 3.0, 4.0, 5.0, 6.0, 7.0, or 8.0 or greater with a lower 95% confidence interval limit of greater than 1. In one embodiment, the predisposing polymorphic allele is associated with AMD with an odds ratio of at least 2 and a lower 95% confidence limit greater than 1. Methods for determining an odds ratio are well known in the art (see, for example, Schlesselman et al., Case Control Studies: Design, Conduct and Analysis Oxford University Press, New York (1982)).

In one embodiment, alleles associated with infarct size and responsiveness to adenosine agonists have a p value of equal to or less than 0.05. In other embodiments, the p value is equal to or less than 0.01. As used herein, the term “p value” is synonymous with “probability value.” As is well known in the art, the expected p value for the association between a random allele and disease is 1.00. A p value of less than about 0.05 indicates that the allele and disease do not appear together by chance but are influenced by positive factors. Generally, the statistical threshold for significance of linkage has been set at a level of allele sharing for which false positives would occur once in twenty genome scans (p=0.05). In particular embodiments, alleles associated with infarct size and responsiveness to adenosine agonists is associated with infarct size with a p value of equal to or less than 0.1, 0.05, 0.04, 0.03, 0.02, 0.01, 0.009, 0.008, 0.007, 0.006, 0.005, 0.004, 0.003, 0.002 or 0.001, or with a p value of less than 0.00095, 0.0009, 0.00085, 0.0008 or 0.0005. It is recognized that, in some cases, p values may need to be corrected, for example, to account for factors such as sample size (number of families), genetic heterogeneity, clinical heterogeneity, or analytical approach (parametric or nonparametric method).

### Genotyping A1-AR and A3-AR Alleles

According to one aspect of the present invention, a method for determining whether a human is homozygous for a polymorphism, heterozygous for a polymorphism, or lacking the polymorphism altogether (i.e. homozygous wildtype) is encompassed. As an illustrative example only, the Iso248Leu in the A3-AR gene polymorphism, a method for determining the T-allele, heterozygous for the T- and C-alleles, or homozygous for the T-allele of the human A3-AR gene is provided. Substantially any method of detecting any allele of the A3-AR and/or A1-AR gene (including coding and no-coding regions such as the 3′UTR) gene, such as hybridization, amplification, restriction enzyme digestion, and sequencing methods, can be used.

In one embodiment, a haplotyping method useful according to the present invention is a physical separation of alleles by cloning, followed by sequencing. Other methods of haplotyping, useful according to the present invention include, but are not limited to monoallelic mutation analysis (MAMA) (Papadopoulos et al. (1995) Nature Genet. 11:99-102) and carbon nanotube probes (Woolley et al. (2000) Nature Biotech. 18:760-763). U.S. Patent Application No. US 2002/0081598 also discloses a useful haplotyping method which involves the use of PCR amplification.

Computational algorithms such as expectation-maximization (EM), subtraction and PHASE are useful methods for statistical estimation of haplotypes (see, e.g., Clark, A. G. Inference of haplotypes from PCR-amplified samples of diploid populations. Mol Biol Evol 7, 111-22. (1990); Stephens, M., Smith, N. J. & Donnelly, P. A new statistical method for haplotype reconstruction from population data. Am J Hum Genet 68, 978-89. (2001); Templeton, A. R., Sing, C. F., Kessling, A. & Humphries, S. A cladistic analysis of phenotype associations with haplotypes inferred from restriction endonuclease mapping. II. The analysis of natural populations. Genetics 120, 1145-54. (1988)).

In one embodiment, an allelic discrimination method for identifying the A1-AR and/or the A3-AR genotype of a human can be used. In one embodiment, the allelic discrimination method of the invention involves use of a first oligonucleotide probe which anneals with a target portion of the individual's genome. As an illustrative example only, the target portion comprises a portion of the region of A3-AR gene to be screened, for example, including the nucleotide residue at position 1509 in SEQ ID NO: 2. Because the nucleotide residue at this position differs, for example at position in the T-allele and the C-allele, the first probe is completely complementary to only one of the two alleles. Alternatively, a second oligonucleotide probe can also be used which is completely complementary to the target portion of the other of the two alleles. The allelic discrimination method of the invention also involves use of at least one, and preferably a pair of amplification primers for amplifying a reference region of the A3-AR gene of a subject. The reference region includes at least a portion of the human A3-AR, for example a portion including the nucleotide residue at position 1509 of the A3-AR gene in SEQ ID NO: 2. In other embodiments, the methods can be applied to variations or sequence differences of the A1-AR gene and other mutations and/or polymorphisms of the invention.

The probe is preferably a DNA oligonucleotide having a length in the range from about 20 to about 40 nucleotide residues, preferably from about 20 to about 30 nucleotide residues, and more preferably having a length of about 25 nucleotide residues. In one embodiment, the probe is rendered incapable of extension by a PCR-catalyzing enzyme such as Taq polymerase, for example by having a fluorescent probe attached at one or both ends thereof. Although non-labeled oligonucleotide probes can be used in the kits and methods of the invention, the probes are preferably detectably labeled. Exemplary labels include radionuclides, light-absorbing chemical moieties (e.g. dyes), fluorescent moieties, and the like. Preferably, the label is a fluorescent moiety, such as 6-carboxyfluorescein (FAM), 6-carboxy-4,7,2′,7′-tetrachlorofluoroscein (TET), rhodamine, JOE (2,7-dimethoxy-4,5-dichloro-6-carboxyfluorescein), HEX (hexachloro-6-carboxyfluorescein), or VIC.

In a particularly preferred embodiment, the probe of the invention comprises both a fluorescent label and a fluorescence-quenching moiety such as 6-carboxy-N,N,N′,N′-tetramethylrhodamine (TAMRA), or 4-(4′-dimethlyaminophenylazo)benzoic acid (DABCYL). When the fluorescent label and the fluorescence-quenching moiety are attached to the same oligonucleotide and separated by no more than about 40 nucleotide residues, and preferably by no more than about 30 nucleotide residues, the fluorescent intensity of the fluorescent label is diminished. When one or both of the fluorescent label and the fluorescence-quenching moiety are separated from the oligonucleotide, the intensity of the fluorescent label is no longer diminished. Preferably, the probe of the invention has a fluorescent label attached at or near (i.e. within about 10 nucleotide residues of) one end of the probe and a fluorescence-quenching moiety attached at or near the other end. Degradation of the probe by a PCR-catalyzing enzyme releases at least one of the fluorescent label and the fluorescence-quenching moiety from the probe, thereby discontinuing fluorescence quenching and increasing the detectable intensity of the fluorescent labels. Thus, cleavage of the probe (which, as discussed above, is correlated with complete complementarity of the probe with the target portion) can be detected as an increase in fluorescence of the assay mixture.

If detectably different labels are used, more than one labeled probe can be used. For example, the assay mixture can contain a first probe which is completely complementary to the target portion of the polymorphism of A3-AR gene and to which a first label is attached, and a second probe which is completely complementary to the target portion of the wildtype allele. When two probes are used, the probes are detectably different from each other, having, for example, detectably different size, absorbance, excitation, or emission spectra, radiative emission properties, or the like. For example, a first probe can be completely complementary to the target portion of the polymorphism and have FAM and TAMRA attached at or near opposite ends thereof. The first probe can be used in the method of the invention together with a second probe which is completely complementary to the target portion of the wildtype allele and has TET and TAMRA attached at or near opposite ends thereof. Fluorescent enhancement of FAM (i.e. effected by cessation of fluorescence quenching upon degradation of the first probe by Taq polymerase) can be detected at one wavelength (e.g. 518 nanometers), and fluorescent enhancement of TET (i.e. effected by cessation of fluorescence quenching upon degradation of the second probe by Taq polymerase) can be detected at a different wavelength (e.g. 582 nanometers).

Ideally, the probe exhibits a melting temperature (Tm) within the range from about 60° C. to 70° C., and more preferably in the range from 65° C. to 67° C. Furthermore, because each probe is completely complementary to only one of the alleles of the A3-AR gene, each probe will necessarily have at least one nucleotide residue which is not complementary to the corresponding residue of the other allele. This non-complementary nucleotide residue of the probe is preferably located near the midsection of the probe (i.e. within about the central third of the probe sequence) and is preferably approximately equidistant from the ends of the probe. As an illustrative example, the probe which is completely complementary to the polymorphic allele of A3-AR gene can, for example, be completely complementary to nucleotide residues surrounding position 1509 of the polymorphic allele, as defined by the positions of SEQ ID NO:2. For example, because the C- and A-alleles differ at position 1509, this probe will have a mismatched base pair nine nucleotide residues from one end when it is annealed with the corresponding target portion of the C-allele.

By way of example, labeled probes having the sequences of SEQ ID NO:1 can be used, in conjunction with labeled probes having the sequences of SEQ ID NO:2 in order to determine the allelic content of an individual (e.g. to assess whether the mammal comprises one or both of an C allele and a T allele of A3-AR at position 1509). For example, custom TAQMAN® SNP genotyping probes for each allele can be designed using PRIMER EXPRESS® v2.0 software (Applied Biosystems) using recommended guidelines. Successful discrimination of each allele can be verified using population control individuals. Genomic DNA (e.g. 20 ng) can be amplified according to assay recommendations and genotyping analysis performed, as described in greater detail below.

The size of the reference portion which is amplified according to the allelic discrimination method of the invention is preferably not more than about 100 nucleotide residues. It is also preferred that the Tm for the amplified reference portion with the genomic DNA or fragment thereof be in the range from about 57° C. to 61° C., where possible.

It is understood that binding of the probe(s) and primers and that amplification of the reference portion of the A3-AR gene according to the allelic discrimination method of the invention will be affected by, among other factors, the concentration of Mg⁺⁺ in the assay mixture, the annealing and extension temperatures, and the amplification cycle times. Optimization of these factors requires merely routine experimentation which are well known to skilled artisans.

Another allelic discrimination method suitable for use in the present invention employs “molecular beacons”. Detailed description of this methodology can be found in Kostrikis et al., Science 1998; 279:1228-1229, which is incorporated herein by reference.

The use of microarrays comprising a multiplicity of reference sequences is becoming increasingly common in the art. Accordingly, another aspect of the invention comprises a microarray having at least one oligonucleotide probe, as described above, appended thereon.

It is understood, however, that any method of ascertaining an allele of a gene can be used to assess the genotype of the 3′UTR of the A1-AR gene and the coding region of A3-AR gene in a mammal. Thus, the invention includes known methods (both those described herein and those not explicitly described herein) and allelic discrimination methods which may be hereafter developed.

As used herein, a first region of an oligonucleotide “flanks” a second region of the oligonucleotide if the two regions are adjacent one another or if the two regions are separated by no more than about 1000 nucleotide residues, and preferably no more than about 100 nucleotide residues.

A second set of primers is “nested” with respect to a first pair of primers if, after amplifying a nucleic acid using the first pair of primers, each of the second pair of primers anneals with the amplified nucleic acid, such that the amplified nucleic acid can be further amplified using the second pair of primers.

Nucleic acid molecules of the present invention may be prepared by two general methods: (1) Synthesis from appropriate nucleotide triphosphates, or (2) Isolation from biological sources. Both methods utilize protocols well known in the art.

The availability of nucleotide sequence information, such as a full length nucleic acid sequence having SEQ ID NO: 1 and SEQ ID NO:2, enables preparation of isolated nucleic acid molecules of the invention by oligonucleotide synthesis. Synthetic oligonucleotides may be prepared by the phosphoramidite method employed in the Applied Biosystems 38A DNA Synthesizer or similar devices. The resultant construct may be purified according to methods known in the art, such as high performance liquid chromatography (HPLC). Long, double-stranded polynucleotides, such as a DNA molecule of the present invention, must be synthesized in stages, due to the size limitations inherent in current oligonucleotide synthetic methods. Thus, for example, a 1.4 kb double-stranded molecule may be synthesized as several smaller segments of appropriate complementarity. Complementary segments thus produced may be annealed such that each segment possesses appropriate cohesive termini for attachment of an adjacent segment. Adjacent segments may be ligated by annealing cohesive termini in the presence of DNA ligase to construct an entire 1.4 kb double-stranded molecule. A synthetic DNA molecule so constructed may then be cloned and amplified in an appropriate vector.

Nucleic acid sequences of the present invention may also be isolated from appropriate biological sources using methods known in the art.

Also contemplated with the scope of the present invention are vectors or plasmids containing the nucleic acid sequence of SEQ ID NO:1 and SEQ ID NO:2, and host cells or animals containing such vectors or plasmids. Also encompassed within the scope of the present invention are vectors or plasmids containing the nucleic acid sequences of portions of the nucleic acid sequences of SEQ ID NO:1 and SEQ ID NO:2, comprising the variants of the 3′UTR of A1-AR and/or the A3-AR coding region as disclosed in this invention, and host cells or animals containing such vectors or plasmids. Methods for constructing vectors or plasmids containing the nucleic acid sequence of SEQ ID NO:1 and SEQ ID NO:2, and host cells or animals containing the same are within the ability of persons skilled in the art of molecular biology.

### SNPs, Polymorphisms and Alleles

The genomes of all organisms undergo spontaneous mutation in the course of their continuing evolution, generating variant forms of progenitor genetic sequences (Gusella, Ann. Rev. Biochem. 55, 831-854 (1986)). The coexistence of multiple forms of a genetic sequence gives rise to genetic polymorphisms, including SNPs.

Approximately 90% of all polymorphisms in the human genome are SNPs. SNP refer to single base positions in DNA at which different alleles, or alternative nucleotides, exist in a population. A SNP (interchangeably referred to herein as SNP, SNP site, or SNP locus) is usually preceded by and followed by highly conserved sequences of the allele (e.g., sequences that vary in less than 1/100 or 1/1000 members of the populations). An individual may be homozygous or heterozygous for an allele at each polymorphism or SNP position. A SNP can, in some instances, be referred to as a “cSNP” to denote that the nucleotide sequence containing the SNP is an amino acid coding sequence.

A SNP or polymorphism may arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP or polymorphism can also be a single base insertion or deletion variant referred to as an “indel” (Weber et al., “Human diallelic insertion/deletion polymorphisms”, Am J Hum Genet October 2002; 71(4):854-62).

A synonymous codon change, or silent mutation/SNP or polymorphism (the terms “SNP” and “mutation” are used herein interchangeably), is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. While SNPs or polymorphism can be bi-, tri-, or tetra-allelic, the vast majority of the SNPs are bi-allelic, and are thus often referred to as “bi-allelic markers”, or “di-allelic markers”.

As used herein, references to SNPs and SNP genotypes or polymorphisms include individual SNPs and/or haplotypes, which are groups of SNPs that are generally inherited together. Haplotypes can have stronger correlations with diseases or other phenotypic effects compared with individual SNPs, and therefore may provide increased diagnostic accuracy in some cases (Stephens et al. Science 293, 489-493, 20 Jul. 2001).

Causative SNPs or polymorphisms are those that produce alterations in gene expression or in the expression, structure, and/or function of a gene product, and therefore are most predictive of a possible clinical phenotype. One such class includes SNPs falling within regions of genes encoding a polypeptide product, i.e. cSNPs. These SNPs or polymorphisms can result in an alteration of the amino acid sequence of the polypeptide product (i.e., non-synonymous codon changes) and give rise to the expression of a defective or other variant protein. Furthermore, in the case of nonsense mutations, a SNP or polymorphism can lead to premature termination of a polypeptide product. Such variant products can result in a pathological condition, e.g., genetic disease. Examples of genes in which a SNP or polymorphism within a coding sequence causes a genetic disease include sickle cell anemia and cystic fibrosis.

Causative SNPs or polymorphisms do not necessarily have to occur in coding regions; causative SNPs can occur in, for example, any genetic region that can ultimately affect the expression, structure, and/or activity of the protein encoded by a nucleic acid. Such genetic regions include, for example, those involved in transcription, such as SNPs in transcription factor binding domains, SNPs in promoter regions, in areas involved in transcript processing, such as SNPs at intron-exon boundaries that may cause defective splicing, or SNPs in mRNA processing signal sequences such as polyadenylation signal regions. Some SNPs that are not causative SNPs nevertheless are in close association with, and therefore segregate with, a disease-causing sequence. In some situations, the presence of a SNP or polymorphism correlates with the presence of, or predisposition to, or an increased risk in developing the disease. These SNPs, although not causative, are nonetheless also useful for diagnostics, disease predisposition screening, and other uses.

An association study of a SNP or polymorphism and a specific disorder involves determining the presence or frequency of the SNP allele or polymorphism allele in biological samples from subjects with the disorder of interest, such as coronary artery disease or coronary syndrome, and comparing the information to that of controls (i.e., individuals who do not have the disorder; controls may be also referred to as “healthy” or “normal” individuals) who are preferably of similar age and race. The appropriate selection of patients and controls is important to the success of SNP association studies. Therefore, a pool of individuals with well-characterized phenotypes is extremely desirable.

In some embodiments, a SNP or polymorphism can be screened in diseased tissue samples or any biological sample obtained from a diseased individual, and compared to control samples, and selected for its increased (or decreased) occurrence in a specific pathological condition, such as pathologies related to coronary artery disease and coronary syndrome. Once a statistically significant association is established between one or more SNP(s) or polymorphisms and a pathological condition (or other phenotype) of interest, then the region around the SNP or polymorphism can optionally be thoroughly screened to identify the causative genetic locus/sequence(s) (e.g., causative SNP/mutation, gene, regulatory region, etc.) that influences the pathological condition or phenotype. Association studies may be conducted within the general population and are not limited to studies performed on related individuals in affected families (linkage studies).

In some embodiments, SNP alleles, sometimes referred to as polymorphisms or polymorphic alleles, of the present invention can be associated with a risk of having a small or large infarction. In some embodiments the infarction is myocardial infarction. In other embodiments, the SNPs, polymorphisms or polymorphic alleles, of the present invention can be associated with a risk of having increased or diminished responsiveness to adenosine and adenosine receptor agonists. Mutations or alleles that are associated with a risk of having a small infarction and decreased responsiveness to adenosine receptor agonist may be referred to as protective” alleles, and mutations and/or alleles, and SNP alleles that are associated with an increased risk of having a large infarction and increased responsiveness to adenosine receptor agonists may be referred to as “susceptibility” alleles or “risk factors”. Thus, whereas certain SNPs (or their encoded products) can be assayed to determine whether a subject possesses a SNP allele or polymorphism allele that is indicative of a risk of having a large infarction or having increased responsiveness to adenosine agonists (i.e., a susceptibility allele), other SNPs (or their encoded products) can be assayed to determine whether a subject possesses a SNP allele or polymorphism allele that is indicative of a risk of small infarction and having diminished responsiveness to adenosine receptor agonists (i.e., a protective allele). Similarly, particular SNP alleles or polymorphism alleles of the present invention can be associated with either an increased or decreased likelihood of responding to a particular treatment or therapeutic compound, or an increased or decreased likelihood of experiencing toxic effects from a particular treatment or therapeutic compound. The term “altered” may be used herein to encompass either of these two possibilities (e.g., an increased or a decreased risk/likelihood).

Those skilled in the art will readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference may be made to either strand in order to refer to a particular SNP position, SNP allele, or nucleotide sequence. Probes and primers, may be designed to hybridize to either strand and SNP genotyping methods disclosed herein may generally target either strand. Throughout the specification, in identifying a SNP position or polymorphism, reference is generally made to the protein-encoding strand, only for the purpose of convenience.

Nucleic acids. Certain embodiments of the present invention concern various nucleic acids, including promoters, amplification primers, oligonucleotide probes and other nucleic acid elements involved in the analysis of genomic DNA. In certain aspects, a nucleic acid comprises a wild type, a mutant, or a polymorphic nucleic acid.

1. Preparation of Nucleic Acids A nucleic acid may be made by any technique known to one of ordinary skill in the art, such as for example, chemical synthesis, enzymatic production or biological production. Non limiting examples of a synthetic nucleic acid (e.g. a synthetic oligonucleotide), include a nucleic acid made by in vitro chemical synthesis using phosphodiester, phosphite, or phosphoramidite chemistry and solid phase techniques such as described in European Patent 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotides may be used.

Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid includes one produced by enzymes in amplification reactions such as PCR (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

2. Purification of Nucleic Acids A nucleic acid may be purified on polyacrylamide gels, cesium chloride centrifugation gradients, chromatography columns or by any other means known to one of ordinary skill in the art (see for example, Sambrook et al., 2001, incorporated herein by reference).

In certain aspects, the present invention concerns a nucleic acid that is an isolated nucleic acid. As used herein, the term “isolated nucleic acid” refers to a nucleic acid molecule (e.g. an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

3. Nucleic Acid Segments. In certain embodiments, the nucleic acid is a nucleic acid segment. As used herein, the term “nucleic acid segment” refers to fragments of a nucleic acid, such as, for a non-limiting example, those that encode only part of a A1-AR or A3-AR gene sequence. Thus, a “nucleic acid segment” may comprise any part of a gene sequence, including from about 2 nucleotides to the full length gene including regulatory regions to the polyadenylation signal and any length that includes all the coding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created: n to n+y where n is an integer from 1 to the last number of the sequence and y is the length of the nucleic acid segment minus one, where n y does not exceed the last number of the sequence.

Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 and so on. For a 20-mer, the nucleic segments correspond to bases 1 to 20, 2 to 21, 3 to 22 and so on. In certain embodiments, the nucleic acid segment may be a probe or primer. As used herein, a “probe” generally refers to a nucleic acid used in a detection method or composition.

As used herein, a “primer” generally refers to a nucleic acid used in an extension or amplification method or composition.

4. Nucleic Acid Complements The present invention also encompasses a nucleic acid that is complementary to a nucleic acid. A nucleic acid “complement(s)” or is “complementary” to another nucleic acid when it is capable of base-pairing with another nucleic acid according to the standard Watson-Crick, Hoogsteen, or reverse Hoogsteen binding complementarily rules. As used herein “another nucleic acid” may refer to a separate molecule or a spatially separated sequence of the same molecule. In preferred embodiments, a complement is a hybridization probe or amplification primer for the detection of a nucleic acid polymorphism.

As used herein, the term “complementary” or “complement” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. However, in some diagnostic or detection embodiments, completely complementary nucleic acids are preferred.

Nucleic acid detection. Some embodiments of the invention concern identifying polymorphisms in A1-AR and/or A3-AR gene, correlating genotype or haplotype to phenotype, wherein the phenotype is lowered or altered A1-AR RNA stability and/or lowered function of A3-AR activity or expression. Thus, the present invention involves assays for identifying polymorphisms and other nucleic acid detection methods. Nucleic acids, therefore, have utility as probes or primers for embodiments involving nucleic acid hybridization. They may be used in diagnostic or screening methods of the present invention.

Detection of nucleic acids encoding A1-AR and/or A3-AR gene as well as nucleic acids involved in the expression or stability of A1-AR and/or the A3-AR polypeptides or transcripts, are encompassed by the invention. General methods of nucleic acid detection are provided below, followed by specific examples employed for the identification of polymorphisms, including single nucleotide polymorphisms (SNPs).

1. Hybridization The use of a probe or primer of 3, 4, 5, 6, 7, 8, 9, 10, 11, 12, 13, 14, 15, 16, 17, 18, 19, 20, 21, 22, 23, 24, 25, 50, 60, 70, 80, 90, or 100 nucleotides, preferably at least 17 and nucleotides in length, or in some aspects up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more I complementary sequences of 20 to 30 nucleotides, or even longer where desired. Such fragments may be readily prepared, for example, by directly synthesizing the fragment by chemical means or by introducing selected sequences into recombinant vectors for recombinant production.

In certain embodiments, the probe or primer comprises 7, 8, 9, 10, 11, 12, 13, 14, 15, 16, 17, 18, 19, 20, 21, 22, 23, 24, 25, 50, 60, 70, 80, 90, or 100 consecutive nucleotides of SEQ ID NO:1 or SEQ NO:2. Accordingly, the nucleotide sequences of the invention can be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting a specific polymorphism. It is generally appreciated that conditions can be rendered more stringent by the addition of increasing amounts of formamide. For example, under highly stringent conditions, hybridization to filter-bound DNA may be carried out in 0.5 M NaHPO, 7% sodium dodecyl sulfate (SDS), 1 mM EDTA at 65° C., and washing in 0.1×SSC/0.1% SDS at 68° C. (Ausubel et al., 1996).

Conditions may be rendered less stringent by increasing salt concentration and/or decreasing temperature. For example, a medium stringency condition could be provided by about 0.1 to 0.25M NaCl at temperatures of about 37° C. to about 55° C., while a low stringency condition could be provided by about 0.15M to about 0.9M salt, at temperatures ranging from about 20° C. to about 55° C. Under low stringent conditions, such as moderately stringent conditions the washing may be carried out for example in 0.2×SSC/0.1% SDS at 42° C. (Ausubel et al., 1996). Hybridization conditions can be readily manipulated depending on the desired results.

In other embodiments, hybridization may be achieved under conditions of, for example, 50 mM Tris-HCl (pH 5.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl2, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In some embodiments, one may desire to employ a fluorescent label or an enzyme tag such as urease, alkaline phosphatase, or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples. In other aspects, a particular nuclease cleavage site may be present and detection of a particular nucleotide sequence can be determined by the presence or absence of nucleic acid cleavage.

In general, it is envisioned that the probes or primers described herein will be useful as reagents in solution hybridization, as in PCR, for detection of expression or genotype of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626.

Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids Nucleic acids used as a template for amplification may be isolated from cells, tissues or other samples according to standard methodologies (Sambrook et al., 2001). In certain embodiments, analysis is performed on whole cell or tissue homogenates or biological fluid samples with or without substantial purification of the template nucleic acid. The nucleic acid may be genomic DNA or fractionated or whole cell RNA. Where RNA is used, it may be desired to first convert the RNA to a complementary DNA.

Depending upon the desired application, high stringency hybridization conditions may be selected that will only allow hybridization to sequences that are completely complementary to the primers. In other embodiments, hybridization may occur under reduced stringency to allow for amplification of nucleic acids that contain one or more mismatches with the primer sequences. Once hybridized, the template-primer complex is contacted with one or more enzymes that facilitate template dependent nucleic acid synthesis. Multiple rounds of amplification, also referred to as “cycles,” are conducted until a sufficient amount of amplification product is produced. The amplification product may be detected, analyzed or quantified. In certain applications, the detection may be performed by visual means. In certain applications, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR) which is described in detail in U.S. Pat. Nos. 4,683,195, 4,683,202 and 4,800,159, and in Tnnis et al., 1988, each of which is incorporated herein by reference in their entirety.

Another method for amplification is ligase chain reaction (“LCR”), disclosed in European Application No. 320,308, and is incorporated herein by reference in its entirety. U.S. Pat. No. 4,883,750 describes a method similar to LCR for binding probe pairs to a target sequence. A method based on PCR and oligonucleotide ligase assay (OLA) (described in further detail below), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, Great Britain Application 2 202 328, and in PCT Application PCT/US89/01025, each of which is incorporated herein by reference in its entirety. Qbeta Replicase, described in PCT Application PCT/US87/00880, may also be used as an amplification method in the present invention.

An isothermal amplification method, in which restriction endonucleases and ligases are used to achieve the amplification of target molecules that contain nucleotide 5′-[alpha-thio] triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety).

European Application 329 822 disclose a nucleic acid amplification process involving cyclically synthesizing single-stranded RNA (“ssRNA”), ssDNA, and double-stranded DNA (dsDNA), which may be used in accordance with the present invention. PCT Application WO 89/06700 (incorporated herein by reference in its entirety), discloses a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1994; Ohara et al., 1989).

3. Detection of Nucleic Acids following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid. Separation of nucleic acids may also be effected by spin columns and/or chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized, with or without separation. A typical visualization method involves staining of a gel with ethidium bromide and visualization of bands under W light. Alternatively, if the amplification products are integrally labeled with radio- or fluorometrically-labeled nucleotides, the separated amplification products card be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis I and blotting without external manipulation of the gel and is ideally suited to carrying out methods according to the present invention.

Other methods of nucleic acid detection that may be used in the practice of the invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

### Other Assays

Other methods for genetic screening may be used within the scope of the present invention, for example, to detect mutations in genomic DNA, cDNA and/or RNA samples.

Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR (see above), single strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded I RNA/RNA, RNA/DNA or DNA/DNA molecule. This definition thus includes mismatches due to insertion/deletion mutations, as well as single or multiple base point mutations.

U.S. Pat. No. 4,946,773 describes an RNaseA mismatch cleavage assay that involves annealing single-skanded DNA or RNA test samples to an RNA probe, and subsequent treatment of the nucleic acid duplexes with RNaseA. For the detection of mismatches, the single-stranded products of the RNaseA treatment, electrophoretically separated according to size, are compared to similarly treated control duplexes. Samples containing smaller fragments (cleavage products) not seen in the control duplex are scored as positive.

Other investigators have described the use of RNaseI in mismatch assays. The use of RNaseI for mismatch detection is described in literature from Promega Biotech. Promega markets a kit containing RNaseI that is reported to cleave three out of four known mismatches.

Others have described using the MutS protein or other DNA-repair enzymes for detection of single-base mismatches. Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

Specific Examples of SNP Screening Methods Spontaneous mutations that arise during the course of evolution in the genomes of organisms are often not immediately transmitted throughout all of the members of the species, thereby creating polymorphic alleles that co-exist in the species populations. Often polymorphisms are the cause of genetic diseases. Several classes of polymorphisms have been identified. For example, variable nucleotide type polymorphisms (VNTRs), arise from spontaneous tandem duplications of di- or trinucleotide repeated motifs of nucleotides. If such variations or sequence differences alter the lengths of DNA fragments generated by restriction endonuclease cleavage, the variations are referred to as restriction fragment length polymorphisms (RFLPs). RFLPs are widely used in human and animal genetic analyses.

Another class of polymorphisms are generated by the replacement of a single nucleotide.

Such single nucleotide polymorphisms (SNPs) rarely result in changes in a restriction endonuclease site. Thus, SNPs are rarely detectable by restriction fragment length analysis. SNPs are the most common genetic variations or sequence differences and occur once every 100 to 300 bases and several SNP mutations have been found that affect a single nucleotide in a protein-encoding gene in a manner sufficient to actually cause a genetic disease. SNP diseases are exemplified by hemophilia, sickle-cell anemia, hereditary hemochromatosis, late-onset Alzheimer's disease etc. In context of the present invention, polymorphic mutations that affect the activity and/or levels of the A1-AR and/or A3-AR gene products will be determined by any of a series of screening methods. One set of screening methods is aimed at identifying SNPs that affect the inducibility, activity and/or level of the A1-AR and/or A3-AR gene products in in vitro or in viva assays. The other set of screening methods will then be performed to screen an individual for the occurrence of the SNPs identified above. To do this, a sample (such as blood or other bodily fluid or tissue sample) will be taken from a subject for genotype analysis.

SNPs can be the result of deletions, point mutations and insertions. In general any single base alteration, whatever the cause, can result in a SNP. The greater frequency of SNPs means that they can be more readily identified than the other classes of polymorphisms. The greater uniformity of their distribution permits the identification of SNPs “nearer” to a particular trait of interest. The combined effect of these two attributes makes SNPs extremely valuable. For example, if a particular trait (e.g., destabilization of the A1-AR RNA or dysfunction of A3-AR) reflects a mutation at a particular locus, then any polymorphism that is linked to the particular locus can be used to predict the probability that an individual will exhibit that trait. In some cases, the SNP or polymorphism may be the cause of the trait.

Several methods have been developed to screen polymorphisms and some examples are listed below. The reference of Kwok and Chen (2003) and Kwok (2001) provide overviews of some of these methods, both of these references are specifically incorporated by reference.

SNPs relating to the regulation of A1-AR stability and/or A3-AR function can be characterized by the use of any of these methods or suitable modification thereof. Such methods include the direct or indirect sequencing of the site, the use of restriction enzymes where the respective alleles of the site create or destroy a restriction site, or the use of allele-specific hybridization probes.

Examples of identifying polymorphisms and applying that information in a way that yields useful information regarding patients can be found, for example, in U.S. Pat. No. 6,472,157; U.S. Patent Application Publications 20020016293, 20030099960, 20040203034; WO 0180896, all of which are hereby incorporated by reference.

a) DNA Sequencing The most commonly used method of characterizing a polymorphism is direct DNA I sequencing of the genetic locus that flanks and includes the polymorphism. Such analysis can be accomplished using either the “dideoxy-mediated chain termination method,” also known as the “Sanger Method” (Sanger et al., 1975) or the “chemical degradation method,” also known as the “Maxam-Gilbert method” (Maxam et al., 1977). Sequencing in combination with genomic sequence-specific amplification technologies, such as the polymerase chain reaction may be i utilized to facilitate the recovery of the desired genes (Mullis et al., 1986; European Patent Application 50,424; European Patent Application. 84,796, European Patent Application 258,017, European Patent Application. 237,362; European Patent Application. 201,184; U.S. Pat. Nos. 4,683,202; 4,582,788; and 4,683,194), all of the above incorporated herein by reference.

b) Exonuclease Resistance. Other methods that can be employed to determine the identity of a nucleotide present at a polymorphic site utilize a specialized exonuclease-resistant nucleotide derivative (U.S. Pat. No. 4,656,127). A primer complementary to an allelic sequence immediately 3′- to the polymorphic site is hybridized to the DNA under investigation. If the polymorphic site on the DNA contains a nucleotide that is complementary to the particular exonucleotide-resistant nucleotide derivative present, then that derivative will be incorporated by a polymerase onto the end of the hybridized primer. Such incorporation makes the primer resistant to exonuclease cleavage and thereby permits its detection. As the identity of the exonucleotide-resistant derivative is known one can determine the specific nucleotide present in the polymorphic site of the DNA.

c) Microsequencing Methods. Several other primer-guided nucleotide incorporation procedures for assaying polymorphic sites in DNA have been described (Komher et al., 1989; Sokolov 1990, Syvanen 1990; Kuppuswamy et al., 1991; Prezant et al., 1992; Ugozzoll et al, 1992; Nyren et al., 1993). These methods rely on the incorporation of labeled deoxynucleotides to discriminate between bases at a polymorphic site. As the signal is proportional to the number of deoxynucleotides incorporated, polymorphisms that occur in runs of the same nucleotide result in a signal that is proportional to the length of the run (Syvanen et al., 1990).

d) Extension in Solution. French Patent 2,650,840 and PCT Application WO91/02087 discuss a solution-based method for determining the identity of the nucleotide of a polymorphic site. According to these methods, a primer complementary to allelic sequences immediately 3′- to a polymorphic site is used. The identity of the nucleotide of that site is determined using labeled dideoxynucleotide derivatives which are incorporated at the end of the primer if complementary to the nucleotide of the polymorphic site.

e) Genetic Bit Analysis or Solid-Phase Extension. PCT Application WO92/15712 describes a method that uses mixtures of labeled terminators and a primer that is complementary to the sequence 3′ to a polymorphic site. The labeled terminator that is incorporated is complementary to the nucleotide present in the polymorphic site of the target molecule being evaluated and is thus identified. Here the primer or the target molecule is immobilized to a solid phase.

f) Oligonucleotide Ligation Assay (OLA) This is another solid phase method that uses different methodology (Landegren et al., 1988). Two oligonucleotides, capable of hybridizing to abutting sequences of a single strand of a target DNA are used. One of these oligonucleotides is biotinylated while the other is detectably labeled. If the precise complementary sequence is found in a target molecule, the oligonucleotides will hybridize such that their termini abut, and create a ligation substrate. Ligation permits the recovery of the labeled oligonucleotide by using avidin. Other nucleic acid detection assays, based on this method, combined with PCR have also been described (Nickerson et al., 1990). Here PCR is used to achieve the exponential amplification of target DNA, which is then detected using the OLA.

g) Ligase/Polymerase-Mediated Genetic Bit Analysis. U.S. Pat. No. 5,952,174 describes a method that also involves two primers capable of hybridizing to abutting sequences of a target molecule. The hybridized product is formed on a solid support to which the target is immobilized. Here the hybridization occurs such that the primers are separated from one another by a space of a single nucleotide. Incubating this hybridized product in the presence of a polymerase, a ligase, and a nucleoside triphosphate mixture containing at least one deoxynucleoside triphosphate allows the ligation of any pair of abutting hybridized oligonucleotides. Addition of a ligase results in two events required to generate a signal, extension and ligation. This provides a higher specificity and lower “noise” than methods using either extension or ligation alone and unlike the polymerase-based assays, this method enhances the specificity of the polymerase step by combining it with a second hybridization and a ligation step for a signal to be attached to the solid phase.

h) Invasive Cleavage Reactions Invasive cleavage reactions can be used to evaluate cellular DNA for a particular polymorphism. A technology called INVADER™ Technology employs such reactions (e.g. de Arruda et al., 2002; Stevens et al., 2003, which are incorporated by reference). Generally, there are three nucleic acid molecules: 1) an oligonucleotide upstream of the target site (“upstream oligo”), 2) a probe oligonucleotide covering the target site (“probe”), and 3) a single-stranded DNA with the target site (“target”). The upstream oligo and probe do not overlap but they contain contiguous sequences. The probe contains a donor fluorophore, such as fluoroscein, and an acceptor dye, such as Dabcyl. The nucleotide at the 3′ terminal end of the upstream oligo overlaps (“invades”) the first base pair of a probe-target duplex. Then the probe is cleaved by a structure-specific 5′ nuclease causing separation of the fluorophore/quencher pair, which increases the amount of fluorescence that can be detected. See Lu et al., 2004. In some cases, the assay is conducted on a solid-surface or in an array format.

h) Other Methods To Detect SNPs. Several other specific methods for SUP detection and identification are presented below and may be used as such or with suitable modifications in conjunction with identifying polymorphisms of the A1-AR and A3-AR genes in the present invention. Several other methods are also described on the SNP web site of the NCBI at the website www.ncbi.nlm.nih.gov, incorporated herein by reference.

In a particular embodiment, extended haplotypes may be determined at any given locus in a population, which allows one to identify exactly which SNPs will be redundant and which will be essential in association studies. The latter is referred to as ‘haplotype tag SNPs (htSNPs)’, markers that capture the haplotypes of a gene or a region of linkage disequilibrium. See Johnson et al. (2001) and Ke and Cardon (2003), each of which is incorporated herein by reference, for exemplary methods.

The VDA-assay utilizes PCR amplification of genomic segments by long PCR methods using TaKaRa LA Taq reagents and other standard reaction conditions. The long amplification can amplify DNA sizes of about 2,000-12,000 bp. Hybridization of products to variant detector array (VDA) can be performed by an Affymetrix High Throughput Screening Center and analyzed with computerized software.

A method called Chip Assay uses PCR amplification of genomic segments by standard or long PCR protocols. Hybridization products are analyzed by VDA, Halushka et al., 1999, i incorporated herein by reference. SNPs are generally classified as “Certain” or “Likely” based on computer analysis of hybridization patterns. By comparison to alternative detection methods such as nucleotide sequencing, “Certain” SNPs have been confirmed 100% of the time; and; “Likely” SNPs have been confirmed 73% of the time by this method.

Other methods simply involve PCR amplification following digestion with the relevant restriction enzyme. Yet others involve sequencing of purified PCR products from known genomic regions.

In yet another method, individual exons or overlapping fragments of large exons are PCR-amplified. Primers are designed from published or database sequences and PCR amplification of genomic DNA is performed using the following conditions: 200 ng DNA template, 0.5 μM each primer, 80 μM each of dCTP, dATP, dTTP and dGTP, 5% formamide, 1.5 mM MgCl₂, 0.5U of Taq polymerase and 0.1 volume of the Taq buffer. Thermal cycling is performed and resulting PCR-products are analyzed by PCR-single strand conformation polymorphism (PCR-SSCP) analysis, under a variety of conditions, e.g. 5 or 10% polyacrylamide gel with 15% urea, with or without 5% glycerol. Electrophoresis is performed overnight. PCR-products that show mobility shifts are reamplified and sequenced to identify nucleotide variation.

In a method called CGAP-GAI (DEMIGLACE), sequence and alignment data (from a PHRAP.ace file), quality scores for the sequence base calls (from PHRED quality files), distance information (from PHYLIP dnadist and neighbour programs) and base-calling data (from PHRED ‘-d’ switch) are loaded into memory. Sequences are aligned and examined for each vertical chunk (‘slice’) of the resulting assembly for disagreement. Any such slice is considered a candidate SNP (DEMIGLACE). A number of filters are used by DEMIGLACE to eliminate slices that are not likely to represent true polymorphisms. These include filters that: (i) exclude sequences in any given slice from SNP consideration where neighboring sequence quality scores drop 40% or more; (ii) exclude calls in which peak amplitude is below the fifteenth percentile of all base calls for that nucleotide type; (iii) disqualify regions of a sequence having a high number of disagreements with the consensus from participating in SNP calculations; (iv) remove from consideration any base call with an alternative call in which the peak takes up 25% or more of the area of the called peak; (v) exclude variations that occur in only one read direction. PHRED quality scores were converted into probability-of-error values for each nucleotide in the slice. Standard Bayesian methods are used to calculate the posterior probability that there is evidence of nucleotide heterogeneity at a given location.

In a method called CU-RDF (RESEQ), PCR amplification is performed from DNA isolated from blood using specific primers for each SNP, and after typical cleanup protocols to remove unused primers and free nucleotides, direct sequencing using the same or nested primers.

In a method called DEBNICK (METHOD-B), a comparative analysis of clustered EST sequences is performed and confirmed by fluorescent-based DNA sequencing. In a related method, called DEBNICK (METHOD-C), comparative analysis of clustered EST sequences with phred quality can be done where least two occurrences of each allele is performed and confirmed by examining traces.

In a method identified as ERO (RESEQ), new primers sets were designed for electronically published STSs and used to amplify DNA from 10 different mouse strains. The amplification product from each strain is then gel purified and sequenced using a standard dideoxy, cycle sequencing technique with 33P-labeled terminators. All the ddATP terminated reactions are then loaded in adjacent lanes of a sequencing gel followed by all of the ddGTP reactions and so on. SNPs are identified by visually scanning the radiographs.

In another method identified as ERO (RESEQ-HT), new primers sets were designed for electronically published murine DNA sequences and used to amplify DNA from 10 different mouse strains. The amplification product from each strain is prepared for sequencing by treating with Exonuclease I and Shrimp Alkaline Phosphatase. Sequencing is performed using ABI Prism Big Dye Terminator Ready Reaction Kit (Perkin-Elmer) and sequence samples are run on the 3700 DNA Analyzer (96 Capillary Sequencer).

FGU-CBT (SCA2-SNP) identifies a method where the region containing the SNP is PCR amplified using the primers SCA2-FP3 and SCA2-RP3. Approximately 100 ng of genomic DNA is amplified in a 50 ml reaction volume containing a final concentration of 5 mM Tris, 25 mM KCl, 0.75 mM MgCl2, 0.05% gelatin, 20 pmol of each primer and 0.5U of Taq DNA polymerase. Samples are denatured, annealed and extended and the PCR product is purified from a band cut out of the agarose gel using, for example, the QIAquick gel extraction kit (Qiagen) and is sequenced using dye terminator chemistry on an ABI Prism 377 automated DNA sequencer with the PCR primers.

In a method identified as JBLACK (SEQ/RESTRICT), two independent PCR reactions are performed with genomic DNA. Products from the first reaction are analyzed by sequencing, indicating a unique FspI restriction site. The mutation is confirmed in the product of the second PCR reaction by digesting with FspI. In a method described as KWOK(1), SNPs are identified by comparing high quality genomic sequence data from four randomly chosen individuals by direct DNA sequencing of PCR products with dye-terminator chemistry (see Kwok et al., 1996). In a related method identified as KWOK (2) SNPs are identified by comparing high quality genomic sequence data from overlapping large-insert clones such as bacterial artificial chromosomes (BACs) or P1 based artificial chromosomes (PACs). An STS containing this SNP is then developed and the existence of the SNP in various populations is confirmed by pooled DNA sequencing (see Taillon-Miller et al., 1998). In another similar method called KWOK(3), SNPs are identified by comparing high quality genomic sequence data from overlapping large-insert clones BACs or PACs. The SNPs found by this approach represent DNA sequence variations between the two donor chromosomes but the allele frequencies in the general population have not yet been determined. In method KWOK(5), SNPs are identified by comparing high quality genomic sequence data from a homozygous DNA sample and one or more pooled DNA samples by direct DNA sequencing of PCR products with dye-terminator chemistry. The STSs used are developed from sequence data found in publicly available databases. Specifically, these STSs are amplified by PCR against a complete hydatidiform mole (CHM) that has been shown to be homozygous at all loci and a pool of DNA samples from 80 CEPH parents (see Kwok et al., 1994).

In another such method, KWOK (OverlapSnpDetectionWithPolyBayes), SNPs are discovered by automated computer analysis of overlapping regions of large-insert human genomic clone sequences. For data acquisition, clone sequences are obtained directly from large-scale sequencing centers. This is necessary because base quality sequences are not present/available through GenBank. Raw data processing involves analysis of clone sequences and accompanying base quality information for consistency. Finished (‘base perfect’, error rate lower than 1 in 10,000 bp) sequences with no associated base quality sequences are assigned a uniform base quality value of 40 (1 in 10,000 bp error rate). Draft sequences without base quality values are rejected. Processed sequences are entered into a local database. A version of each sequence with known human repeats masked is also stored. Repeat masking is performed with the program “MASKERAID.” Overlap detection: Putative overlaps are detected with the program “WUBLAST.” Several filtering steps follow in order to eliminate false overlap detection results, i.e. similarities between a pair of clone sequences that arise due to sequence duplication as opposed to true overlap. Total length of overlap, overall percent similarity, number of sequence differences between nucleotides with high base quality value “high-quality mismatches.” Results are also compared to results of restriction fragment mapping of genomic clones at Washington University Genome Sequencing Center, finisher's reports on overlaps, and results of the sequence contig building effort at the NCBI. SNP detection: Overlapping pairs of clone sequence are analyzed for candidate SNP sites with the ‘POLYBAYES’ SNP detection software. Sequence differences between the pair of sequences are scored for the probability of representing true sequence variation as opposed to sequencing error. This process requires the presence of base quality values for both sequences. High-scoring candidates are extracted. The search is restricted to substitution-type single base pair variations. Confidence score of candidate SNP is computed by the POLYBAYES software.

In a method identified by KWOK (TAQMAN® assay), the TAQMAN® assay is used to determine genotypes for 90 random individuals. In a method identified by KYUGEN(Q1), DNA samples of indicated populations are pooled and analyzed by PLACE-SSCP. Peak heights of each allele in the pooled analysis are corrected by those in a heterozygote, and are subsequently used for calculation of allele frequencies. Allele frequencies higher than 10% are reliably quantified by this method. Allele frequency=0 (zero) means that the allele was found among individuals, but the corresponding peak is not seen in the examination of pool. Allele frequency 0-0.1 indicates that minor alleles are detected in the pool but the peaks are too low to reliably quantify.

In yet another method identified as KYUGEN (Method1), PCR products are post-labeled with fluorescent dyes and analyzed by an automated capillary electrophoresis system under SSCP conditions (PLACE-SSCP). Four or more individual DNAs are analyzed with or without two pooled DNA (Japanese pool and CEPH parents pool) in a series of experiments. Alleles are identified by visual inspection. Individual DNAs with different genotypes are sequenced and SNPs identified. Allele frequencies are estimated from peak heights in the pooled samples after correction of signal bias using peak heights in heterozygotes. The PCR primers are tagged to; have 5P-ATT or 5′-GTT at their ends for post-labeling of both strands. Samples of DNA (10 ng/ul) are amplified in reaction mixtures containing the buffer (10 mM Tris-HCl, pH 8.3 or 9.3, 50 mM KCl, 2.0 mM MgCl2), 0.25 EM of each primer, 200 IBM of each dNTP, and 0.025 units/ul I of Taq DNA polymerase premixed with anti-Taq antibody. The two strands of PCR products are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase.

For the SSCP: an aliquot of fluorescently labeled PCR products and TAMRA-labeled internal markers are added to deionized formamide, and denatured. Electrophoresis is performed in a capillary using an ABI Prism 310 Genetic Analyzer. Genescan softwares (P-E Biosystems) are used for data collection and data processing. DNA of individuals including those who showed different genotypes on SSCP are subjected for direct sequencing using big-dye terminator chemistry, on ABI Prism 310 sequencers. Multiple sequence trace files obtained from ABI Prism 310 are processed and aligned by Phred/Phrap and viewed using Consed viewer. SNPs are identified by PolyPhred software and visual inspection.

In yet another method identified as KYUGEN (Method2), individuals with different genotypes are searched by denaturing HPLC (DHPLC) or PLACE-SSCP (Inazuka et al., 1997) and their sequences are determined to identify SNPs. PCR is performed with primers tagged with 5P-ATT or 5′-GTT at their ends for post-labeling of both strands. DHPLC analysis is carried out using the WAVE DNA fragment analysis system (Transgenomic). PCR products are injected into DNASep column, and separated under the conditions determined using WAVEMaker program (Transgenomic). The two strands of PCR products that are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. SSCP followed by electrophoresis is performed in a capillary using an ABI Prism 310 Genetic Analyzer.

Genescan softwares (P-E Biosystems). DNA of individuals including those who showed different genotypes on DHPLC or SSCP are subjected for direct sequencing using big-dye terminator chemistry, on ABI Prism 310 sequencer. Multiple sequence trace files obtained from; ABI Prism 310 are processed and aligned by PhredlPhrap and viewed using Consed viewer.

SNPs are identified by PolyPhred software and visual inspection. Trace chromatogram data of i EST sequences in Unigene are processed with PHRED. To identify likely SNPs, single base i mismatches are reported from multiple sequence alignments produced by the programs PHRAP, BRO and POA for each Unigene cluster. BRO corrected possible misreported EST orientations, while POA identified and analyzed non-linear alignment structures indicative of gene mixing/chimeras that might produce spurious SNPs. Bayesian inference is used to weigh evidence for true polymorphism versus sequencing error, misalignment or ambiguity, is clustering or chimeric EST sequences, assessing data such as raw chromatogram height, sharpness, overlap and spacing; sequencing error rates; context-sensitivity; cDNA library origin, etc. In method identified as MARSHFIELD (Method-B), overlapping human DNA sequences which contained putative insertion/deletion polymorphisms are identified through searches of public databases. PCR primers which flanked each polymorphic site are selected from the consensus sequences. Primers are used to amplify individual or pooled human genomic DNA. Resulting PCR products are resolved on a denaturing polyacrylamide gel and a PhosphorImager is used to estimate allele frequencies from DNA pools.

6. Linkage Disequilibrium. Polymorphisms in linkage disequilibrium with the polymorphism at 1689, 2205, 2683, −54, 717 in the A1-AR gene, and/or the 1509 A3-AR gene locus may also be used with the methods of the present invention. “Linkage disequilibrium” (“LD” as used herein, though also referred to as “LED” in the art) refers to a situation where a particular combination; of alleles (i.e., a variant form of a given gene) or polymorphisms at two loci appears more frequently than would be expected by chance. “Significant” as used in respect to linkage disequilibrium, as determined by one of skill in the art, is contemplated to be a statistical p or o value that may be 0.25 or 0.1 and may be 0.1, 0.05. 0.001, 0.00001 or less. The relationship between A1-AR and/or A3-AR haplotypes and the expression level of the A1-AR and A3-AR proteins may be used to correlate the genotype (i.e., the genetic make up of an organism) to a phenotype (i.e., the physical traits displayed by an organism or cell). “Haplotype” is used according to its plain and ordinary meaning to one skilled in the art. It refers to a collective genotype of two or more alleles or polymorphisms along one of the homologous chromosomes.

### Solid Supports

Solid supports containing oligonucleotide probes for identifying the alleles, including polymorphic alleles, of the present invention can be filters, polyvinyl chloride dishes, silicon or glass based chips, etc. Such wafers and hybridization methods are widely available, for example, those disclosed by Beattie (WO 95/11755). Any solid surface to which oligonucleotides can be bound, either directly or indirectly, either covalently or noncovalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, about 2, 10, 100, 1000 to 10,000; 100,000, 400,000 or 1,000,000 of such features on a single solid support. The solid support, or the area within which the probes are attached may be on the order of a square centimeter.

Oligonucleotide probe arrays can be made and used according to any techniques known in the art (see for example, Lockchart et al. (1996), Nat. Biotechnol. 14: 1675-1680; McGall et al. (1996), Proc. Nat. Acad. Sci. USA 93: 13555-13460). Such probe arrays may contain at least two or more oligonucleotides that are complementary to or hybridize to two or more of the SNPs described herein. Such arrays may also contain oligonucleotides that are complementary or hybridize to at least about 2, 3, 4, 5, 6, 7, 8, 9, 10, 20, 30, 50 or more SNPs described herein.

Methods of forming high density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling (see Pirrung et al. (1992), U.S. Pat. No. 5,143,854; Fodor et al. (1998), U.S. Pat. No. 5,800,992; Chee et al. (1998), U.S. Pat. No. 5,837,832.

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithographic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in Fodor et al., (1993). WO 93/09668. High density nucleic acid arrays can also be fabricated by depositing premade or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

### Databases

The present invention includes databases containing information concerning polymorphic alleles associated with the coronary artery disease and coronary syndrome, for instance, information concerning polymorphic allele frequency and strength of the association of the allele with myocardial infarction and the like. Databases may also contain information associated with a given polymorphism such as descriptive information about the probability of association of the polymorphism with prediction of clinical phenotype, for example the likelihood of responsiveness to adenosine treatment and/or prediction of infarct size on myocardial infarction. Other information that may be included in the databases of the present invention include, but is not limited to, SNP sequence information, descriptive information concerning the clinical status of a tissue sample analyzed for SNP haplotype, or the subject from which the sample was derived. The database may be designed to include different parts, for instance a SNP frequency database and a SNP sequence database. Methods for the configuration and construction of databases are widely available, for instance, see Akerblom et al., (1999) U.S. Pat. No. 5,953,727, which is herein incorporated by reference in its entirety.

The databases of the invention may be linked to an outside or external database. In a preferred embodiment, the external database may be the HGBASE database maintained by the Karolinska Institute, The SNP Consortium (TSC) and/or the databases maintained by the National Center for Biotechnology Information (NCBI) such as GenBank.

Any appropriate computer platform may be used to perform the necessary comparisons between polymorphic allele frequency and associated disorder and any other information in the database or provided as an input. For example, a large number of computer workstations are available from a variety of manufacturers, such as those available from Silicon Graphics. Client-server environments, database servers and networks are also widely available and appropriate platforms for the databases of the invention.

The databases of the invention may also be used to present information identifying the polymorphic alleles in a subject and such a presentation may be used to predict the likelihood that the subject will develop AMD. Further, the databases of the present invention may comprise information relating to the expression level of one or more of the genes associated with the polymorphic alleles of the invention.

The polymorphisms identified by the present invention may be used to analyze the expression pattern of an associated gene and the expression pattern correlated to the probability of developing an AMD. The expression pattern in various tissues can be determined and used to identify tissue specific expression patterns, temporal expression patterns and expression patterns induced by various external stimuli such as chemicals or electromagnetic radiation.

### Kits

Any of the compositions described herein may be comprised in a kit. In a non-limiting example, reagents for determining the genotype of one or both A1-AR and/or A3-AR genes are included in a kit. The kit may further include individual nucleic acids that can amplify and/or detect particular nucleic acid sequences the A1-AR and/or A3-AR genes. In specific embodiments, it includes one or more primers and/or probes. Nucleic acid molecules may have a label, dye, or other signaling molecule attached to it, such as a fluorophore. It may also include one or more buffers, such as a DNA isolation buffers, an amplification buffer or a hybridization buffer. The kit may also contain compounds and reagents to prepare DNA templates and isolate DNA from a sample.

The kit may also include various labeling reagents and compounds. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

A kit will also include instructions for employing the kit components as well the use of any other reagent not included in the kit. Instructions may include variations that can be implemented.

The present invention also provides a kit for performing the instant method disclosed herein. The kit comprises a plurality of reagents useful for performing the disclosed methods, and optionally further comprises an instructional material which describes how the method is performed.

For simplicity and by way of an exemplary example only, the kits outlined below describe methods for exemplary kit for performing the allelic discrimination on one of the variances of the invention. For example, the kits described enable allelic discrimination of the 1509(1033)A/C polymorphism in the coding region of the A3-AR gene, and is intended as an illustrative example only, and in no way is it to be limiting the use of these methods and kits to detect only the 1509(1033)A/C variance, the methods are equally applicable for use for allelic discrimination of the other variances of the invention including; 1689(1278)C/A and 2205(1798)Tdel of the 3′UTR of A1-AR gene and 2683(2777)del36 polymorphism on A3-AR gene, as well as −54C/T and 717(716) in the 5′UTR and coding region of the A1-AR gene.

Such exemplary kit for determining the allelic discrimination method of the invention comprises: a) a first oligonucleotide probe which anneals specifically with a target portion of the mammal's genome, wherein the target portion includes the nucleotide residue located at a polymorphic position of SEQ ID NO:2, such as position 1509, the probe comprising a fluorescent label and a fluorescence quencher attached to separate nucleotide residues thereof, and b) a primer for amplifying a reference portion of corresponding wildtype allele of the A3-AR gene, the reference portion including the corresponding non-polymorphic (or wildtype) nucleotide residue, as defined by the sequence of SEQ ID NO: 2.

The kit may further comprise a DNA polymerase having 5′→3′ exonuclease activity. The kit may also comprise a second oligonucleotide probe having a different annealing specificity than the first (e.g. wherein the first is completely complementary to the target portion of the C-allele at position 1509 of SEQ ID NO:2 and the second is completely complementary to the target portion of the A-allele at position 1509 of SEQ ID NO:2), a second primer (e.g. such that this and the other primer can be used to amplify at least the target portion by a PCR), or both. The kit may comprise an instructional material which can, for example, describe performance of the allelic discrimination method, the association between the presence of the C-allele and susceptibility to an increased likelihood to a have a large infarction as well as likelihood of the subject having increased responsiveness to adenosine agonist treatment.

In an alternative embodiment of the present invention, the kit comprises at least one, and preferably two molecular beacon probes, as described herein. When the kit comprises two molecular beacon probes, one is preferably specific for (i.e. completely complementary to a region including the polymorphic nucleotide residue of SEQ ID NO: 2, e.g. nucleotide 1509) the polymorphic allele of the A3-AR gene, and the other is specific for the non-polymorphic allele. This kit may further comprise an instructional material, including a publication, a recording, a diagram, or any other medium of expression which can be used to communicate the usefulness of the composition of the invention for performing a method of the invention or for associating the presence of a polymorphic allele of the A3-AR gene in a subject with susceptibility to developing or having a large infarct size and likelihood of increased responsiveness to adenosine agonist treatment. The instructional material of the kit of the invention can, for example, be affixed to a container which contains a kit of the invention or be shipped together with a container which contains the kit. Alternatively, the instructional material can be shipped separately from the container with the intention that the instructional material and the kit be used cooperatively by the recipient.

Also provided by the present invention are kits for predicting the responsiveness of a subject to adenosine treatment and also likelihood of size of infarct on ischemic damage according to the one or more of the methods of the invention. The kit comprises a plurality of reagents useful for performing one of the methods as described above, and optionally further comprises an instructional material which describes how the method is performed and the association between the presence of a polymorphic allele and responsiveness to adenosine agonist treatment and susceptibility to a large or small infarct size.

Although the foregoing disclosure is principally directed to kits and methods which are applicable to human A1-AR 3′UTR and the coding region of A3-AR, it will be understood by the skilled artisan that such methods and kits are generally applicable to mammals of all sorts. Modification, where necessary, of the kits and methods of the invention to conform to non-human animals and non-human infarction is well understood, and the ordinarily skilled veterinary worker can design and perform such modification with merely ordinary, if any, experimentation. Representative mammals include, for example, primates, cattle, pigs, horses, sheep, cats, and dogs.

### Methods of Treatment

The present invention also provides methods for treatment of a subject who has been determined to carry a variance in the 3′UTR human A1-AR gene and/or coding region of the A3-AR gene that confers a likelihood of a having a large infarction, for example on ischemic damage or a myocardial infarction. In one embodiment, the subject has not yet expressed any symptoms of coronary syndrome or coronary artery disease. In another embodiment, the subject expresses symptoms of coronary syndrome or coronary artery disease and/or has had a myocardial infarction.

The term “adenosine therapy” or “adenosine receptor agonists” are used interchangeably herein to refer to use of any treatment that acts as adenosine, adenosine analogues and mimetics and variants thereof, adenosine receptor agonists, selective adenosine agonists and dual activating adenosine agonists and variants and analogues thereof. Adenosine receptor agonists are also intended to refer to treatment that increase endogenous adenosine levels and/or increase the expression of the A1-adenosine receptor and/or A3-AR. Other adenosine agonists are known to those of skill in the art and are useful in the treatment methods of the invention.

In some embodiments, treatment can include prophylaxis, including agents which slow or prevent the infarction. In other embodiments, the treatments is any means to activate the adenosine pathway and/or adenosine receptors In some embodiments, the treatment is an adenosine or adenosine analogue, for example orally available adenosine analogues, or injectable form of adenosine, such as ADENOSCAN®. In some embodiments, the treatments is any means to activate the adenosine pathway and/or adenosine receptors. In some embodiments, the treatment is an adenosine or adenosine analogue, for example orally available adenosine analogues. In other embodiments, the treatment is an adenosine receptor agonist. For example, the adenosine receptor agonist may be an A1-AR selective agonist, for example 2-chloro-N6-CyClopentyladenosine (CCPA), N6-cyclohexyladenosine (CHA) and adenosine amine congener (ADAC). In other embodiments, the adenosine receptor agonist may be an A3-AR selective agonist, for example N6-(3-isolbenzyl)adenosine-51-N-methyluronamide (IB-MECA), and CI_IB_MECA, MRS584, MRS537, MRS1340 and DBXMA. In an alternative embodiment, the adenosine receptor agonist may be a compound that activates the A1 and A3 receptors simultaneously, for example MRS646 and MRS1364 (see U.S. Pat. No. 9,850,047 which is incorporated herein by reference).

Alternatively, adenosine agonists that are A1-, A2- and/or A3-receptors agonists are encompassed for use in the invention, as well as any adenosine agonists that simultaneously activates any combination or all of the A1, A2 and A3 adenosine receptors, for example the A1/A2 adenosine receptor agonist, such as AMP579 (see Patent Application 2004020248928, which is specifically incorporated herein by reference.

Alternative adenosine agonists are well know to persons skilled in the art and include adenosine agonists or pharmaceutically acceptable derivative is selected from the group consisting of, but not limited to AB-MECA V6-4-amino benzyl-5′-N-methylcarboxamidoadenosine), CPA (N6-cyclopentyladenosine), ADAC (N6-[4-[[[4-[[[(2-aminoethyl)amino]carbonyl]methyl]-anilino]carbonyl]methyl]phenyl]adenosine), CCPA (2-chloro-N6-cyclopentyladenosine), CHA (N6-cyclohexyladenosine), GR79236 (IV6-[1S,trans,2-hydroxycyclopentyl]adenosine), S-ENBA ((2S)—N6-(2-endonorbanyl)adenosine), IAB-MECA (1V6-(4-amino-3-iodobenzyl)adenosine-5′-N-methylcarboxamidoadenosine), R-PIA (R—N6-(phenylisopropyl)adenosine), ATL146e (4-[3-[6-amino-9-(5-ethylcarbamoyl-3,4 dihydroxy-tetrahydro-furan-2-yl)-9H-purin-2-yl]-prop-2-ynyl]-cyclohexanecarboxylic acid methyl ester), CGS-21680 (APEC or 2-Lp-(2-carbonyl-ethyl)-phenyl ethyl amino]-5′-N-ethylcarboxamidoadenosine), CV1808 (2-phenylaminoadenosine), HENECA (2-hex-1-ynyl-5′-N-ethylcarboxamido adenosine), NECA (5′-N-ethyl-carboxamido adenosine), PAPA-APEC (2-(4-[2-[(4-aminophenyl)methyl carbonyl]ethyl]phenyl)ethylamino-5′-N-ethyl carboxamidoadenosine), DITC APEC(2-[p-(4-isothiocyanatophenylaminothiocarbonyl-2-ethyl)-phenylethylamino]-15′-N-ethylcarboxamidoadenosine), DPMA (N6-(2(3,5-dimethoxy phenyl)-2-(2-methyl phenyl)ethyl)adenosine), S-PHPNECA ((S)-2-phenylhydroxypropynyl-5′-N ethylcarbox amidoadenosine), WRC-0470 (2 cyclohexylmethylidenehydrazinoadenosine), AMP-579 (1S-[1a,2b,3b, 4a(S*)]]-4-[7[[2-(3-chloro-2-thienyl)-1-methylpropyl]amino]-3H-imidazo[4,5-b]pyridyl-3-yl]cyclopentane carboxamide), IB-MECA (N-6-(3-iodobenzyl)adenosine −5′-N methyluronamide), 2-CIADO (2-chloroadenosine), I-ABA (N6-(4-amino-3-1 iodobenzyl)adenosine), S-PIA (S-N6-(phenylisopropyl)adenosine), 2-[(2-aminoethyl aminocarbonylethyl)phenylethyl amino]-5′-N-ethyl-carboxamidoadenosine, 2-C1-IB MECA (2-chloro-Ni-(3-iodobenzyl)adenosine-5′-N-methyluronamide), polyadenylic acid, and any mixture thereof.

In alternative embodiments, the subject is administered a treatment or therapeutic compound that functions through the activation of adenosine pathway, and includes compounds already known by persons skilled in the art and compounds that have yet to be developed.

In a further aspect, a method of preventing organ ischemia-reperfusion injury is provided that includes administering to a subject identified to have susceptibility alleles or any subject with at least any allele of 2683(277)del36-54C/T, 717(716)T/G in the A1-AR gene, or 1509(1033)A/C of the A3-AR gene, an adenosine agonist or a pharmaceutically acceptable derivative or prodrug or metabolite thereof.

In one embodiment, the method includes administrating to a subject with at least any allele for 2683(277)del36-54C/T, 717(716)T/G in the A1-AR gene, or 1509(1033)A/C allele of the A3-AR gene a pharmaceutical composition comprising adenosine, or an adenosine agonist or a pharmaceutically acceptable derivative or prodrug or metabolite thereof at the point on or about reperfusion, or before or during the ischemic or injury-inducing event. The organ or tissue injury is related to at least one of cardiac surgery, non-surgical cardiac revascularization, organ transplantation, perfusion, ischemia, reperfusion, ischemia-reperfusion injury, oxidant injury, cytokine induced injury, shock induced injury, resuscitations injury and apoptosis. The shock induced injury can be hemorrhagic, septic, or traumatic injury, or any combination of them.

### Measurement of Biological Activity of Function of A1-AR and A3-AR

In some embodiments, agents that activate A1-AR and/or A3-AR can be used to identify if a sequence difference in SEQ ID NO:1 and SEQ ID NO:2 affect function of the A1-AR and A3-AR. Factors relate to agonist properties are the intrinsic efficacy (E) and the equilibrium dissociation constant of the agonist-receptor complex (K_{d}).

Therefore, a sequence difference which affects the function of A1-AR and/or A3-AR as disclosed herein is a function of both the stimulus produced by a specific A1-adenosine agonist agent interaction with the receptor and the efficiency of the transduction of that stimulus by the tissue. Stimulus is proportional to the intrinsic efficacy of the agent and the number of receptors. Consequently, variation in receptor density in different tissues can affect the stimulus for response.

In other words, the distribution or ratio of A1-AR to A3-AR in the heart will affect how a subject will respond to a pharmaceutical composition comprising at least one agent that activates A1-AR and A3-AR.

Furthermore, some tissues have very efficiently coupled receptors and other relatively inefficient coupled receptors. This has been termed ‘receptor reserve’ (or spare receptor) since in the first case, a maximum effect can be achieved when a relatively small fraction of the receptor is apparently occupied and, further receptor occupancy can produce no additional effect. The magnitude of the response will thus depend on the intrinsic efficacy value so that, by classical definition, full agonists (E=1) produce the maximum response for a given tissue, partial agonists produce a maximum response that is below that induced by the full agonist (0≦E≦1), and antagonists produce no visible response and block the effect of agonists (E=0). These activities can be completely dependent upon the tissue, i.e., upon the efficiency coupling. Therefore, low-efficacy adenosine agonists may be partial agonists in a given tissue and yet full agonists in peripheral arteries with respect to a function such as vasodilation.

The presence of spare receptors in a tissue increases sensitivity to an agonist. Thus, an important biologic consequence of spare receptors is that they allow agonists with low efficacy for receptors to produce full responses at low concentrations and therefore elicit a selective tissue response.

In some embodiments, the methods as disclosed herein allow for identifying and determining sequence differences in the nucleic acid for A1-AR corresponding to SEQ ID NO:1 and sequence differences in the nucleic acid for A3-AR corresponding to SEQ ID NO:3 affect the function of the A1-AR and A3-AR respectively. In some embodiments, the binding affinity of the A₁-AR activating agent can be determined. Compounds identified by this method will demonstrate partial agonist effects in the cAMP assays and a low IC as determined by affinity binding assays.

For example, one can measure the effect of signal transduction, for example increase in cAMP by a adenosine agonist by a A3-AR that has a nucleic acid sequence difference as compared to a A3-AR corresponding to SEQ ID NO:2. Alternatively, one can measure the effect of signal transduction, for example increase in cAMP by a adenosine agonist by a A3-AR that has a change in the amino acid sequence as compared to a A3-AR corresponding to SEQ ID NO:3.

In some embodiments, function of the A3-AR can be determined by comparing the effect of the adenosine agonist on the A3-AR with a sequence difference as compared to wild-type A3-AR corresponding to SEQ ID NO:2 or SEQ ID NO:3. The function of the A3-AR can be monitored by measuring the intrinsic efficacy (E) and the equilibrium dissociation constant of the agent-receptor complex (K_{d}) of the adenosine agonist.

Intrinsic efficacy (maximal efficacy) is the maximum effect that an agonist can produce if the dose is taken to its maximum. Efficacy is determined mainly by the nature of the receptor and its associated effector system. By definition, partial agonist has a lower maximal efficacy than full agonists.

The K_{d} of a drug is obtained from data generated from a saturation experiment analyzed according to a Scatchard plot (B/F versus F), which leads to a linear curve. The K_{d} is estimated as the negative reciprocal of the slope of the line of best fit, and Bₘₐₓ by the abscissa intercept of the line. The reciprocal of K_{d} measures the affinity constant (Kₐ) of the radioligand for the receptor. Thus, for a given ligand-receptor pair, the smaller the K_{d} (0.1-10 nM) the higher its affinity. Bₘₐₓ is expressed as pmol or fmol per mg tissue or protein.

When the saturation experiment is performed in the presence of a displacer (competitor), the line of best fit of the Scatchard plot can be modified in a manner that depends on the type of receptor interaction exhibited by the displacer. Two main cases exist: (1) decreased slope and unchanged Bₘₐₓ, the displacement is of the competitive type; (2) unchanged slope and unchanged displacement of the non-competitive type. Intermediate cases where both the slope and Bₘₐₓ are modified also exist.

Data generated from a displacement experiment are generally fitted by a sigmoidal curve termed the displacement or inhibition curve, that is the percentage radiolabeled ligand specifically bound versus log [displacer] in M). The abscissa of the inflexion point of the curve gives the IC₅₀ value, the concentration of displacer that displaces or inhibitor 50% of the radioactive ligand specifically bound. IC₅₀ is a measure of the inhibitor or affinity constant (Kᵢ) of the displacer for the receptor. IC₅₀ and Kᵢ are linked as follows if the displacement is of the competitive type then:

Kᵢ=IC₅₀/(1+[C*]/K_{d}*

This is the Cheng-Prusoff equation (Biochem. Pharmacol, 22:3099 (1973)). [C*] is the concentration of radioligand and K_{d}* is its dissociation constant. The duration of the biological effect of an agonist is directly related to the binding affinity of a compound. It is desirable that compounds useful in the methods as disclosed herein act as adjuncts have an effect that is long enough to produce a response without repeated administration but short enough to avoid adverse side effects.

The potency is the dose or concentration required to bring about some fraction of a compound's maximal effect (i.e., the amount of compound needed to produce a given effect). In graded dose-response measurements, the effect usually chosen is 50% of the maximum effect and the dose causing the effect is called the EC₅₀. Dose-response ratios using EC₅₀ values for an agonist for a given receptor in the absence and presence of various concentrations of an antagonist for the same receptor are determined and used to construct a Schild plot from which the K_{b} and _{P}A₂ (−log₁₀K_{b}) values are determined.

The concentration of antagonist that causes 50% inhibition is known as the IC₅₀. IC₅₀ is used to determine the K_{b}, the equilibrium dissociation constant for the antagonist-receptor complex. Thus, K_{b}=[IC₅₀]/1+[A]/K_{A}

Wherein K_{A}=equilibrium dissociation constant for an agonist binding to a receptor (concentration of agonist that causes occupancy of 50% of the receptors) and [A] is the concentration of agonist.

An agent can be potent but have less intrinsic activity than another compound. Relatively potent therapeutic compounds are preferable to weak ones in that lower concentrations produce the desired effect while circumventing the effect of concentration dependent side effects.

The tissue specific factors that determine the effect of an agonist are the number of viable specific receptors in a particular tissue [RT] and the efficiency of the mechanisms that convert a stimulus (S) into an effector response. Thus, there exists for a given tissue, a complex function f(S) that determines the magnitude of the response: Response=ƒ=(S)=[ƒ([A]E[RT])]/([A]+K_{d})

### Method to Determine if a Sequence Difference in A1-AR or A3-AR Affects Function.

Several screening methods for can be used to assess A1-AR and/or A3-AR function, and any such method which are commonly known by persons of ordinary skill in the art can be used in the methods as disclosed herein. In this respect, Numann et al. describe a method, wherein adenosine agonists may be tested in cell cultures with respect to their ion channel binding properties (Numann and Negulescu, Trends Cardiovasc. Med. 11:54-59 (2001)). According to another method, adenosine agonists are tested on isolated and perfused hearts (R. Bessho, D. J. J. Chambers, Thorac Cardiovasc. Surg. 122:993-1003 (2001)). In addition thereto, in vivo testing methods are known, wherein the effect of adenosine agonists on the cardiovascular system is monitored by the means of electrocardiography, magnetic resonance imaging, or echocardiography in living animals (Chu et al., BMC Physiol 1:6-11 (2001); Krupnick et al., J Heart Lung Transplant 21:233-43 (2002)).

Methods to Identify Subjects Amenable to Identifying Sequence Differences in the Ar-AR Gene and/or A3-AR Gene.

In some embodiments, the subject amenable to the methods as disclosed herein are identified to have myocardial infarction, and in some embodiments, the subject is identified to be at risk of myocardial infarction, for example the subject has cardiac dysfunction, or expresses a symptom of coronary syndrome. In some embodiments, a subject has suffered an infarction, for example the subject has ischemic damage or a myocardial infarction. In some embodiments, the subject expresses a symptom of coronary syndrome or coronary artery disease and/or has had a myocardial infarction. In another embodiment, the subject has not yet expressed a symptom of coronary syndrome or coronary artery disease.

Without being bound to theory, myocardial infarction (heart attack) can be a consequence of coronary artery disease. In some instances, coronary artery disease can occur from atherosclerosis, when arteries become narrow or hardened due to cholesterol plaque build-up, with further narrowing occurring from thrombi (blood clots) that form on the surfaces of plaques. Myocardial infarction can occurs when a coronary artery is so severely blocked that there is a significant reduction or break in the blood supply, causing damage or death to a portion of the myocardium (heart muscle). Depending on the extent of the heart muscle damage, the patient may experience significant disability or die as a result of myocardial infarction.

In alternative instances, myocardial infarction can result from a temporary contraction or spasm of a coronary artery. When this occurs, the artery narrows and the blood flow from the artery is significantly reduced or stopped. Though the cause of coronary artery spasm is still unknown, the condition can occur in both normal blood vessels and those partially blocked by plaques.

In some embodiments, subjects amenable to the to the methods as disclosed herein is a subject identified to be at risk of myocardial infarction. Such subjects can be identified based on risk factors commonly known by persons in the art to be associated with myocardial infarction, and include for example subjects with hypertension (high blood pressure), low levels of HDL (high-density lipoproteins), or high levels of LDL (low-density lipoprotein) blood cholesterol or high levels of triglycerides, subjects with a family history of heart disease (especially with onset before age 55), aging men and women, persons with type 1 diabetes, post-menopausal women, obese subjects, subjects who smoke, and subjects with increased stress.

Subjects identified by any method to diagnose myocardial infarction (commonly referred to as a heart attack) commonly known by persons of ordinary skill in the art are amenable to treatment using the methods as disclosed herein, and such diagnostic methods include, for example but are not limited to; (i) blood tests to detect levels of creatine phosphokinase (CPK), aspartate aminotransferase (AST), lactate dehydrogenase (LDH) and other enzymes released during myocardial infarction; (ii) electrocardiogram (ECG or EKG) which is a graphic recordation of cardiac activity, either on paper or a computer monitor. An ECG can be beneficial in detecting disease and/or damage; (iii) echocardiogram (heart ultrasound) used to investigate congenital heart disease and assessing abnormalities of the heart wall, including functional abnormalities of the heart wall, valves and blood vessels; (iv) Doppler ultrasound can be used to measure blood flow across a heart valve; (v) nuclear medicine imaging (also referred to as radionuclide scanning in the art) allows visualization of the anatomy and function of an organ, and can be used to detect coronary artery disease, myocardial infarction, valve disease, heart transplant rejection, check the effectiveness of bypass surgery, or to select patients for angioplasty or coronary bypass graft.

In some embodiments, subjects amenable to the methods as disclosed herein is a subject identified to be presently on adenosine treatment or adenosine therapy, for example a subject on any treatment that acts as adenosine, adenosine analogues and mimetics and variants thereof, adenosine receptor agonists, selective adenosine agonists and dual activating adenosine agonists and variants and analogues thereof.

In such embodiments, adenosine treatment can include prophylaxis, including agents which slow or prevent the infarction. In other embodiments, adenosine treatment is any means to activate the adenosine pathway and/or adenosine receptors. In some embodiments, adenosine treatment is an adenosine or adenosine analogue, for example orally available adenosine analogues, or injectable form of adenosine, such as ADENOSCAN®. In some embodiments, adenosine treatment is any means to activate the adenosine pathway and/or adenosine receptors. In some embodiments, adenosine treatment is an adenosine or adenosine analogue, for example orally available adenosine analogues. In other embodiments, adenosine treatment is an adenosine receptor agonist.

The adenosine agonists used in connection with the treatment methods of the present invention are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

The methods of the present invention allow for the early detection of individuals susceptible to infarction, for example myocardial infarction. In some embodiments, the subject is afflicted with, or at risk of developing coronary syndrome or coronary artery disease. Thus, treatment may be initiated early, e.g. before or at the beginning of the onset of symptoms, for example before the onset of the ischemia or infarction. In alternative embodiments, the treatment may be administered to a subject that has, or is at risk of ischemia reperfusion, for example myocardial infarction. In alternative embodiments, the treatment may be administered prior to, during, concurrent or post onset of ischemia. The dosage required at these early stages will be lower than those needed at later stages of disease where the symptoms are severe. Such dosages are known to those of skill in the art and can be determined by the physician in response to the particular patient.

The following examples are provided to illustrate certain embodiments of the invention. They are not intended to limit in any way the remainder of the disclosure.

The practice of the present invention employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook et al. MOLECULAR CLONING: A LABORATORY MANUAL, 2nd edition (1989); CURRENT PROTOCOLS IN MOLECULAR BIOLOGY (F. M. Ausubel et al. eds. (1987)); the series METHODS IN ENZYMOLOGY (Academic Press, Inc., N.Y.); PCR: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1988)); ANIMAL CELL CULTURE (R. I. Freshney ed. (1987)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); IMMUNOCHEMICAL METHODS IN CELL AND MOLECULAR BIOLOGY (Mayer and Walker, eds., Academic Press, London (1987)); HANDBOOK OF EXPERIMENTAL IMMUNOLOGY, Volumes I-IV (D. M. Weir and C. C. Blackwell, eds. (1986)); MANIPULATING THE MOUSE EMBRYO (Cold Spring Harbor Laboratory Press, Cold Spring Harbor, N.Y. (1986)).

### EXAMPLES

The examples presented herein relate to the identification of variances in the A1-AR and A3-AR genes. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

The inventors herein have discovered that in a large group of subjects enrolled in a National Institutes of Health (NIH)-sponsored STICH trial who have had myocardial infarction that results in discrete identifiable scar or infarction, the presence of mutations in the 3′UTR of the A1-AR gene and in the coding region of the A3-AR gene predicts the size of infarction as measured by sophisticated imaging technology. Further, the inventors show that mutations result decreased function of the A3-AR or in the stabilization of the A1-AR receptor mRNA predict the presence of a smaller scar whereas mutations that results in destabilization of the A1-AR mRNA predict a larger scar. This information enables the prediction of the likelihood of the responsiveness to adenosine therapy and can also be used to predict subjects at greater risk of having a large infarction and a potentially life threatening heart attack and/or myocardial infarction and therefore identifies subjects most amenable to therapeutic agents that increase endogenous adenosine levels and/or increase the expression of the A1-AR.

Methods

Study population. This study is comprised of three groups of unrelated populations. DNA samples from 200 normal controls without a history of cardiovascular disease or diabetes were provided by Genomics Collaborative, Cambridge, Mass. DNA samples were also obtained from 235 patients with non-ischemic cardiomyopathy who were part of a series of 479 patients with heart failure and systolic dysfunction referred to the Cardiomyopathy Clinic at the University of Pittsburgh Medical Center between April 1996 and January 2001 as part of a study of Genetic Risk Assessment of Cardiac Events (GRACE). (McNamara DM, J Am Coll Cardiol 2004; 44; 2019) Samples of peripheral blood were obtained from a subset of individuals who participated in the Surgical Treatment for Ischemic Heart Failure (STICH) trial. Total genomic DNA was extracted from these samples using a genomic DNA extraction kit (Promega). The STICH patients had coronary artery disease and left ventricular dysfunction as evidenced by an ejection fraction of <35%. The Institutional Review Board of Thomas Jefferson University approved the study, and written informed consent was obtained from all participants.

DNA sequencing of PCR products. A subgroup of samples from 50 ischemic heart failure patients, 50 idiopathic heart failure patients, and 50 normal controls was selected for systematic sequencing to detect mutations or polymorphisms in the three adenosine receptors: A1-AR, A2A-AR, and A3-AR. Patients were age-matched and all patients were Caucasian. Each sample underwent PCR amplification and was sequenced individually. Oligonucleotide primers were designed using Primer Express 1.0 software, to cover all six exons of A1-AR, all three exons of A2-AR and both exons of A3-AR, plus 50-100 bps of the flanking intron sequence on both the upstream and downstream sides of each exon. In addition, 2000 bp of the 5′ region upstream of exon 1 was also sequenced. PCR amplification was performed in a 50 μl reaction mixture containing 50 ng genomic DNA and 5 μmol of each primer using RedTaq PCR ready mix (Sigma). All samples were amplified with a GeneAmp PCR system 9700 (PE Applied Biosystems, CA, USA) under the following conditions: After an initial 2-minute denaturation at 94° C., 35 cycles were carried out consisting of 20 seconds at 94° C., 20 seconds at 55° C.-60° C. and 45 seconds at 72° C., followed by a final extension step of 5 minutes at 72° C. After the confirmation of a successful PCR amplification on 2.5% agarose gel, 50 μl of PCR products were purified with QuickStep 2 96-well PCR Purification Kit (Edge Biosystems, Gaithersburg, Md., U.S.A.). 5 to 10 μl double stranded DNA samples (50-100 ng) were sequenced with 3.2 pmol primers identical to PCR primers in both forward and reverse directions. A BigDye Terminator v3.1 Cycle Sequencing Kit from Applied Biosystems was used for sequencing. Standard cycle conditions were: 25 cycles of 96° C. for 10 s, 55° C. for 5 s, 60° C. for 4 min. After cycling and purification, sequencing products were loaded on the 3100 DNA analyzer for capillary electrophoresis and analysis. All DNA sequences were analyzed using ClustalW multiple alignment tool of ChromasPro 1.11 software. Potential mutations were carefully examined and allele frequency was determined in each of the three experimental groups.

Genotyping. The presence of four SNPs identified in the initial sequencing reactions were assessed by high-through put analysis and 8 additional variants were determined by PCR based RFLP method. A1-AR 717 T/G, A2A-AR 1469C/T, A3 1509A/C and A3-AR 1664C/T were determined with TAQMAN® SNP genotyping assays (Assays-on demand and ASSAYS ON DESIGN™) following the manufacturer's instruction (Applied Biosystems). Briefly, PCR amplification was performed in 1×PCR reaction mix in a final volume of 25 μl, containing 10 ng genomic DNA, two unlabeled Gene specific PCR primers and a FAM fluorescent dye-labeled first allele specific probe and a VIC dye labeled second allele specific probe, with a ABI 9700 thermocycler under the following conditions: initial hold for 10 min, 40 cycles of 15 s for 92° C., and 1 min for 62° C. The fluorescent dye was measured post-PCR with the ABI 7900 sequence detection system. To confirm high through-put analysis results, an alternative restriction enzyme method was used. For detection of the A1-AR 717T/G polymorphism PCR was performed with forward primer 5′-ACCCGGAGGTAGAGGTCC-3′ and reverse primer 5′-ATCGCCCTGGTCTCTGTG-3′. After amplification, 20 ul of PCR products were digested with AciI restriction enzyme. The digested products were separated with 3% metaphor agarose gel and visualized by ethidium bromide staining. To detect 2683del36, PCR products were separated with agarose gel without enzyme digestion. The remaining 6 SNPs in the ADORA1gene were genotyped using a standard PCR-based restriction fragment length polymorphism (RFLP)-assays.

Analysis of secondary structure. The effect of individual SNPs on the secondary structure of the mRNA was assessed using the mfold program described by Zuker using default parameters. (Zuker M; 2003; Nucleic Acid Res 31; 3406-3415) and was used to predict ‘local’ secondary structure as described by Chen et al. (Chen J-M, Hum Genet. 2006; 120:301-333).

Population phenotyping. Left ventricular morphology was assessed using standard echocardiograph techniques. Radionuclide imaging studies were performed on a subset of patients enrolled in the STICH trial using either rest/redistribution thallium imaging, stress/redistribution/reinjection thallium imaging, or nitroglycerin-enhanced rest sestamibi SPECT imaging. A semi-quantitative visual assessment of myocardial perfusion was performed on all radionuclide imaging studies (stress, rest, or “viability”) using a 5-point, 17 segment model of the left ventricle, in which a score of 0=normal and a score of 4=absent uptake of tracer. A viability assessment was determined on a particular subject only if he or she had normal resting perfusion (a summed rest score of 0) or if a dedicated viability study was performed as described above. Myocardial viability was determined on a prospective basis based on quantitative analysis of tracer activity. Regional tracer activity was expressed as a percentage of the maximal tracer activity in the myocardium. A myocardial segment was deemed viable if the percent activity of the tracer was is equal to or greater than 50% of the area with the maximal activity. For thallium rest/redistribution imaging, a segment with activity of less than 50% of the maximal myocardial activity on the redistribution images was termed viable if the improvement in the percent activity from the rest to redistribution images was greater than or equal to 12%. Segments were categorized as nonviable or infarcted if they did not meet the viability criteria above. The percent infarcted myocardium for each patient was determined by dividing the number of nonviable segments by the total number of segments.

Statistical Analysis. To assess the presence of an association between the presence of disease and a SNP in the adenosine genes, we used a chi-square test with 4 degrees of freedom in SNPs where all 3 possible genotypes were observed and with 2 degrees of freedom in SNPs where only one homozygous genotype and the heterozygous genotype were observed. Haplotype frequencies were estimated and their differences between the three groups tested using the method described in Schaid, et al (Schaid, D. J (2002. Am J Hum Genet, 70(2):425-434.) The association of each SNP with measures of BNP, cardiac morphology and cardiac function used linear regression, adjusting for age, race and sex. Values were log-transformed to improve the model fit, based on analysis of the residuals.

### Example 1

The inventors have discovered, using direct DNA sequencing in 150 unrelated subjects, 13 variants in the nucleic acid sequence for A1-AR: one SNP in upstream of exon 1 (−54C/T), 2 SNPs upstream of exon 3 (−3751 G/T; −2551 A/G), one SNP upstream of exon 4 (−78C/T), one SNP in the coding region [717(716, 805)T/G], 6 SNPs in the 3′UTR [1689(1278)C/A, 1739(1328)C/T, 1816(1405)C/T, 2038(1627)G/T, 2682(2776)C/T, 2725(2819)T/G], a single nucleotide deletion in the 3′ UTR [2205(1795)Tdel] and a 36 nucleotide deletion [2683(2777)del36] in the 3′UTR. We found 4 SNPs in the coding region of A2A-AR, [818(423)C/T Ala144Ala, 1271(855)C/T Pro295Pro, 1430(1044)C/T Ala348Ala, 1469(1083)C/T Tyr361Tyr]. None of the SNPs in the coding region altered the amino acid sequence of the A2A-AR. In addition, we identified 5 SNPs in the 5′ region [−1622 G/A, −1521G/A, −377A/T, 283G/T], 3 SNPs in 5′UTR [187(−581)G/A, 204(−564)C/T, 364(−404)C/T], 3 SNPs in the coding region [1112(636C/T The115The, 1509(1033)A/C Iso248Leu, 1664(1162)C/T Ala299Ala] and 5SNPs in 3′UTR [1913(1146)C/T, 2060(1293)C/T, 2101(1334)A/G, 2147(1380)G/A, 2165(1398)G/A) in ADORA3. Only the 1509(1033)A/C Iso248Leu SNP resulted in a change in the amino acid sequence of the receptor protein. The nucleotide numbers are based on Ensemble cDNA ID of ENSG00000163485 (SEQ ID NO:1) for A1-AR, ENSG00000128271 for A2A-AR, ENST0000241356 (SEQ ID NO:2) for A3-AR. Numbers in ( ) are based on the numbering of Deckert et al. (Deckert J, Am J Med Gen 81; 18:1988).

### Example 2

To assess whether the presence of a genetic variant was associated with the development of a dilated cardiomyopathy, the inventors analyzed the allele frequency of common SNPs (8 SNPs in A1-AR, 1 SNP in A2-AR, and 2 SNPs in A3-AR) (see Table 1) in DNA from a larger

TABLE 1
Adenosine receptor SNPs (single nucleotide polymorphisms)

| Gene | SNP |
|---|---|
| A1 | 54 C/T, 716 T/G, 1278 C/A, 1328 C/T, 1405 C/T |
| | 1627 G/T, 1795 Tdel, Del36 |
| A2A | 1083 C/T |
| A3 | 1033 A/C, 1162 C/T |

population of 200 normal controls, 230 patients with non-ischemic cardiomyopathy, and 680 patients with ischemic cardiomyopathy.

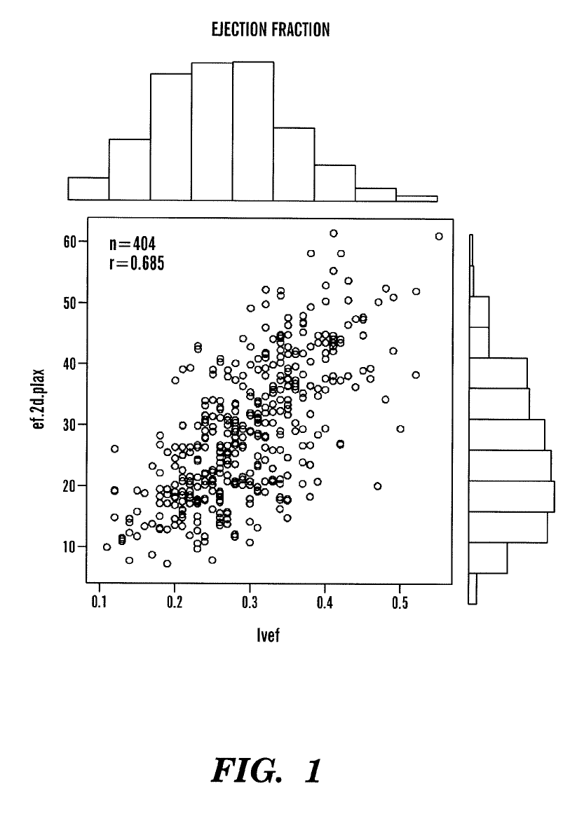

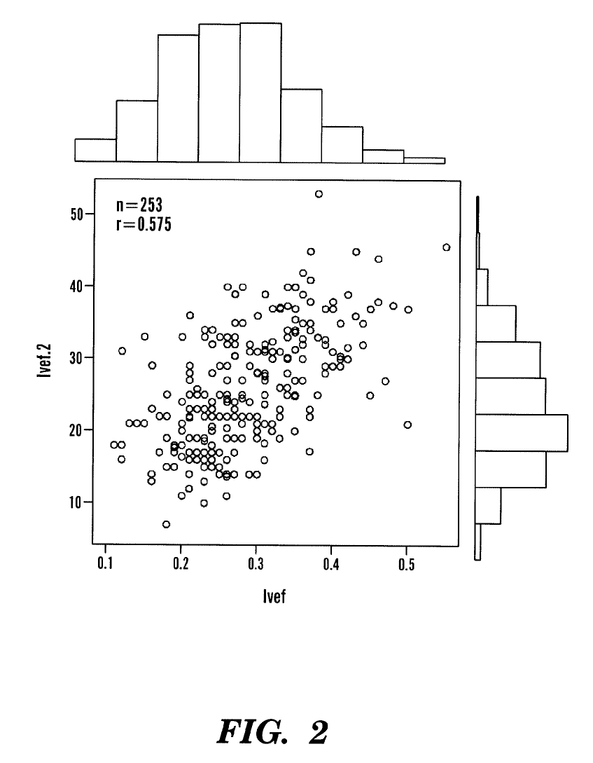

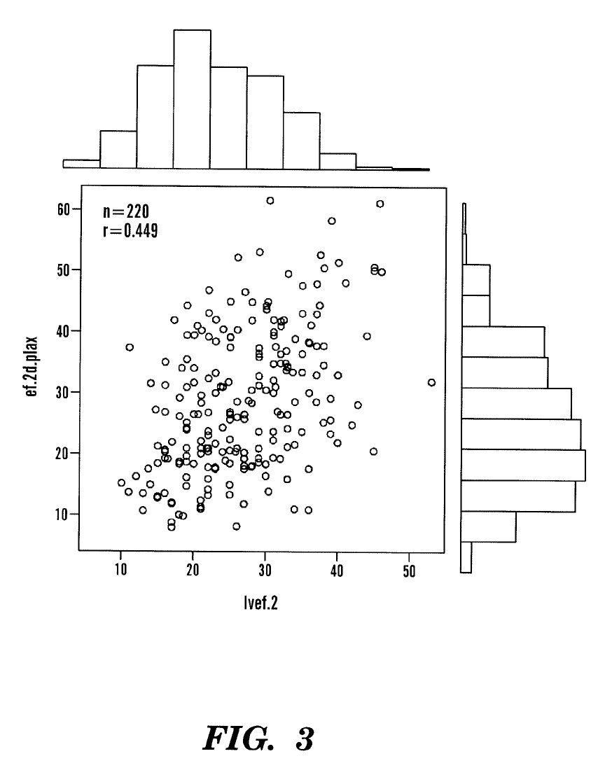

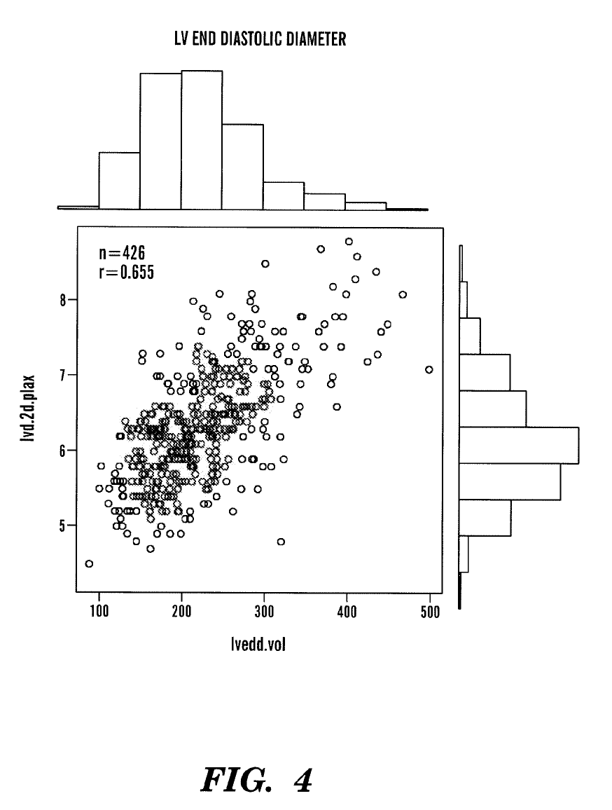

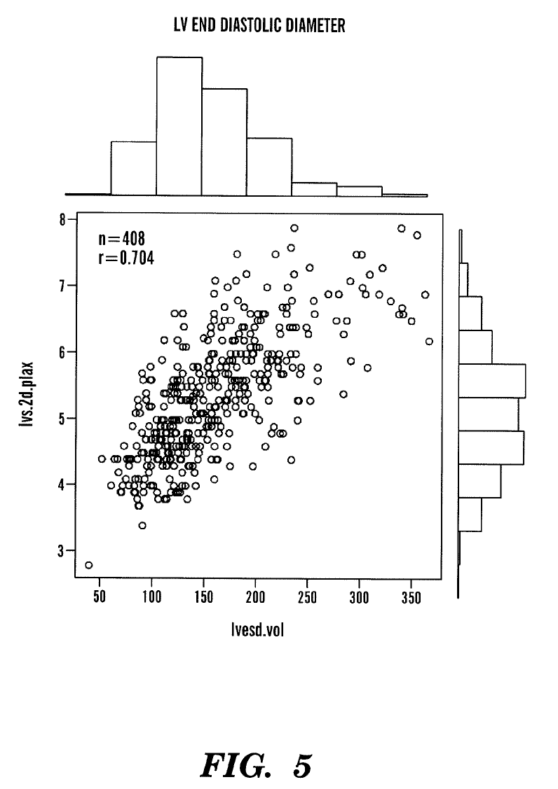

As seen in Table 2, the allele frequency for each SNP did not differ significantly amongst the three groups. The inventors discovered the allele frequencies for both the common and the uncommon polymorphisms in the A1-AR gene was similar to that previously reported in a German population (Deckert J, Am J Med Gen 81; 18:1998) but was significantly different from that reported in a Japanese population. (Ida A J Hum Gen 49; 194; 2004). However, there has been no correlation between the presence of theses polymorphism and a functional change or significance in the individual having such polymorphism. When adjusted for age, sex, and race, the presence of one of the 8 SNPs analyzed in either one or both alleles of the A1-AR gene was not associated with a change in either brain natriuretic peptide levels, left ventricular end-diastolic volume (FIG. 4), left ventricular end-systolic volume (FIG. 5) or ejection fraction (FIGS. 1-3) when compared with the phenotype of individuals having the wild-type genotype.

TABLE 2
Description of A1 SNPs by study group

| | | Control | | Pittsburgh | | STICH | | Combined | | |
|---|---|---|---|---|---|---|---|---|---|---|
| | N | N = 203 | | N = 224 | | N = 768 | | N = 1195 | | Test Statistic |
| a1.54.c.t: 11 | 1181 | 45% | $\frac{89}{197}$ | 42% | $\frac{93}{219}$ | 46% | $\frac{349}{765}$ | 45% | $\frac{531}{1181}$ | χ₄² = 0.83, P = 0.935 |
| 12 | | 44% | $\frac{87}{197}$ | 47% | $\frac{103}{219}$ | 44% | $\frac{334}{765}$ | 44% | $\frac{524}{1181}$ | |
| 22 | | 11% | $\frac{21}{197}$ | 11% | $\frac{23}{219}$ | 11% | $\frac{82}{765}$ | 11% | $\frac{126}{1181}$ | |
| a1.716t.g: 11 | 1184 | 42% | $\frac{82}{197}$ | 45% | $\frac{101}{222}$ | 46% | $\frac{355}{765}$ | 45% | $\frac{538}{1184}$ | χ₄² = 1.65, P = 0.8 |
| 12 | | 47% | $\frac{92}{197}$ | 45% | $\frac{99}{222}$ | 43% | $\frac{328}{765}$ | 44% | $\frac{519}{1184}$ | |
| 22 | | 12% | $\frac{23}{197}$ | 10% | $\frac{22}{222}$ | 11% | $\frac{82}{765}$ | 11% | $\frac{127}{1184}$ | |
| a1.1278.c.a: 11 | 1160 | 94% | $\frac{189}{202}$ | 95% | $\frac{183}{192}$ | 92% | $\frac{706}{766}$ | 93% | $\frac{1078}{1160}$ | χ₂² = 2.46, P = 0.292 |
| 12 | | 6% | $\frac{13}{202}$ | 5% | $\frac{9}{192}$ | 8% | $\frac{60}{766}$ | 7% | $\frac{82}{1160}$ | |
| a1.1328.c.t: 11 | 1187 | 87% | $\frac{174}{200}$ | 94% | $\frac{207}{220}$ | 88% | $\frac{676}{767}$ | 89% | $\frac{1057}{1187}$ | χ₄² = 8.13, P = 0.087 |
| 12 | | 13% | $\frac{26}{200}$ | 6% | $\frac{13}{220}$ | 12% | $\frac{89}{767}$ | 11% | $\frac{128}{1187}$ | |
| 22 | | 0% | $\frac{0}{200}$ | 0% | $\frac{0}{220}$ | 0% | $\frac{2}{767}$ | 0% | $\frac{2}{1187}$ | |
| a1.1405c.t: 11 | 671 | | | | | 93% | $\frac{621}{671}$ | 93% | $\frac{621}{671}$ | |
| 12 | | | | | | 7% | $\frac{50}{671}$ | 7% | $\frac{50}{671}$ | |
| a1.1627g.t: 11 | 1185 | 91% | $\frac{178}{196}$ | 90% | $\frac{201}{224}$ | 88% | $\frac{671}{765}$ | 89% | $\frac{1050}{1185}$ | χ₂² = 1.84, P = 0.399 |
| 12 | | 9% | $\frac{18}{196}$ | 10% | $\frac{23}{224}$ | 12% | $\frac{94}{765}$ | 11% | $\frac{135}{1185}$ | |
| a1.1795.tdel: 11 | 1151 | 93% | $\frac{182}{195}$ | 92% | $\frac{177}{192}$ | 92% | $\frac{706}{764}$ | 93% | $\frac{1065}{1151}$ | χ₂² = 0.23, P = 0.891 |
| 12 | | 7% | $\frac{13}{195}$ | 8% | $\frac{15}{192}$ | 8% | $\frac{58}{764}$ | 7% | $\frac{86}{1151}$ | |
| a1.del36: 11 | 1193 | 97% | $\frac{196}{202}$ | 98% | $\frac{220}{224}$ | 98% | $\frac{753}{767}$ | 98% | $\frac{1169}{1193}$ | χ₂² = 1.13, P = 0.567 |
| 12 | | 3% | $\frac{6}{202}$ | 2% | $\frac{4}{224}$ | 2% | $\frac{14}{767}$ | 2% | $\frac{24}{1193}$ | |

N is the number of non-missing values. Test used: Pearson test

However, as seen in Table 3 and Table 4, the presence of a single allelic SNP at nt 1689(1278)C/A or nt 2205(1795)Tdel in the 3′ UTR of the A1-AR gene was associated with a decrease in infarct size whereas a SNP at nt 2683(2777)del36 in the 3′UTR of the A1-AR gene was associated with an increase in infarct size. No patients harbored a SNP at any of these three sites on both alleles and the prevalence of even the heterozygous condition was small. Interestingly, when analyzed using the mfold program, all three of these SNPs in the 3′ UTR of the A1-AR predicted a significant change in the secondary structure of the RNA. There was also a significant association between an increase in infarct size and a polymorphism in a single allele at either nt −54C/T or in a SNP within the coding region at nt 717(716, 805)T/G; however, an effect was not obvious on infarct size when both alleles harbored the mutation suggesting that these SNPs were not informative. The presence of a relatively common SNP on either one or both alleles in the coding region of the A3-AR gene at nt 1509(1033)A/C Iso248Leu was associated with an increase in infarct size as well as a significant increase in left ventricular end-systolic (log.lvesd; n=547, 1/1 5.015, n=315; 1/2 4.961 n=78; 2/2 5.408 n=6; F-statistic 4.14, p=0.017) and end-diastolic volumes (log.lvedd; 1/1 5.372; 1/2 5.309; 2/2 5.707, F-statistic 5.638, P=0.004) when the SNP was present on both alleles. Importantly, this polymorphism was the only one in all of the AR genes that effected a change in an amino acid.

The data presented herein demonstrate an association between polymorphisms in the adenosine genes and infarct size in a population of patients with a history of coronary artery disease and left ventricular dysfunction. This finding is consistent with earlier studies demonstrating that perturbations of the levels of adenosine receptors can have profound effects on the inherent cardiac protective mechanisms. For example, both the A1- and A3-ARs have been implicated in mediating cardio-protection in animal models of ischemia-reperfusion injury (Headrick J P, Am J Physiol Heart circ Physiol 285; H1797; 2003) and in decreasing infarct size in animal models of acute myocardial infarction. (Yang Z, Am J Physiol Heart Circ. Physiol 282:H949:2002; Guo Y, J Mol Cell Cardiol 33; 825-830, 2001) Furthermore, these findings are consistent with the recognition that adenosine preconditions human myocardium against ischemia in vivo. (Leesar M A, Circulation 1997; 95:2500) That polymorphisms in the adenosine receptors can be associated with altered phenotypes in humans has been shown in studies of patients with neuropsychiatric disease that demonstrate an association between silent mutations in the A2A receptor gene polymorphisms and caffeine-induced anxiety (Alsene K M, Neurpsychopharmacology 28; 1694-1702) and panic disorders (Deckert J, Mol Psychiatry 3:81-85:1998; Hamilton S P, Neuropsychopharmacology 29; 558; 2004). We were unable to demonstrate an effect of the most common A2A polymorphism (1083 C/T) on infarct size; however, the relevance of the A2A-AR in cardiac protection is less clear than for the A1- and A3-ARs.

TABLE 3
Adenosine Receptor SNPs.
Adenosine Receptor SNPs

| SNPs | N | |
|---|---|---|
| a1.54.c.t: 11 | 763 | 46% (348) |
| 12 | | 44% (333) |
| 22 | | 11% (82) |
| a1.716t.g: 11 | 763 | 46% (354) |
| 12 | | 43% (327) |
| 22 | | 11% (82) |
| a1.1278.c.a: 11 | 764 | 92% (704) |
| 12 | | 8% (60) |
| a1.1328.c.t: 11 | 765 | 88% (674) |
| 12 | | 12% (89) |
| 22 | | 0% (2) |
| a1.1405c.t: 11 | 669 | 93% (619) |
| 12 | | 7% (50) |
| a1.1627g.t: 11 | 763 | 88% (669) |
| 12 | | 12% (94) |
| a1.1795.tdel: 11 | 762 | 92% (704) |
| 12 | | 8% (58) |
| a1.del36: 11 | 765 | 98% (751) |
| 12 | | 2% (14) |
| a3.1033a.c: 11 | 544 | 79% (429) |
| 12 | | 19% (106) |
| 22 | | 2% (9) |
| a3.1162c.t: 11 | 643 | 64% (411) |
| 12 | | 31% (199) |
| 22 | | 5% (33) |
| a2a.1083c.t: 11 | 675 | 37% (250) |
| 21 | | 46% (310) |
| 22 | | 17% (115) |

N is the number of non-missing values.
Numbers are after percents are frequencies.

TABLE 4
Association between Adenosine Receptor SNPs and Infarct Size in
Patients with Coronary Artery Disease and Left Ventricle Dysfunction

| SNP | N | 1/1 | n | 1/2 | n | 2/2 | n | F-stat | p value |
|---|---|---|---|---|---|---|---|---|---|
| A₁ 54 c/t | 273 | 0.220 | 124 | 0.240 | 126 | 0.220 | 21 | 3.11 | 0.046 |
| | | | | [0.055] | | | | | 0.015 |
| | | | | | | [0.009] | | | 0.834 |
| A₁ 716 t/g | 273 | 0.215 | 122 | 0.273 | 126 | 0.251 | 23 | 3.30 | 0.038 |
| | | | | [0.058] | | | | | 0.084 |
| | | | | | | [0.430] | | | 0.258 |
| A₁ 1,278 c/a | 273 | 0.251 | 255 | 0.151 | 16 | | | | 0.025 |
| | | | | [−0.104] | | | | | 0.025 |
| A₁ 1,328 c/t | 273 | 0.242 | 235 | 0.255 | 35 | 0.295 | 2 | 0.153 | 0.858 |
| | | | | [0.013] | | | | | 0.693 |
| | | | | | | [0.051] | | | 0.690 |
| A₁ 1,405 c/t | 273 | 0.249 | 251 | 0.196 | 19 | | | 0.863 | 0.284 |
| | | | | [−0.047] | | | | | 0.208 |
| A₁ 1,627 g/t | 273 | 0.245 | 237 | 0.246 | 33 | | | 0.130 | 0.719 |
| | | | | [0.012] | | | | | 0.719 |
| A₁ 1,795 tdel | 273 | 0.250 | 254 | 0.151 | 16 | | | 4.874 | 0.028 |
| | | | | [−0.101] | | | | | 0.028 |
| A₁ del 36 | 273 | 0.241 | 266 | 0.393 | 6 | | | 4.605 | 0.033 |
| | | | | [0.158] | | | | | 0.030 |
| A_{2A} 1,162 c/t | 273 | 0.229 | 89 | 0.243 | 129 | 0.207 | 34 | 0.83 | 0.423 |
| | | | | [−0.008] | | | | | 0.628 |
| | | | | | | [−0.047] | | | 0.140 |
| A₃ 1,033 a/c | 273 | 0.221 | 184 | 0.291 | 37 | 0.388 | 6 | 4.17 | 0.017 |
| | | | | [0.067] | | | | | 0.036 |
| | | | | | | [0.168] | | | 0.037 |
| A₃ 1,162 c/t | 273 | 0.229 | 166 | 0.275 | 87 | 0.229 | 17 | 1.891 | 0.153 |
| | | | | [0.046] | | | | | 0.060 |
| | | | | | | [0.004] | | | 0.937 |

1/1 = wildtype,
1/2 = heterozygous,
2/2 = homozygous for SNP,
F-stat,-F-statistic testing for differences across all possible genotypes.
The effects of individual SNPs in comparison with the wildtype (1/1) are also provided in [ ].

The inventors herein have discovered that a single polymorphism in the A3-AR gene that resulted in a change in the amino acid sequence of the transcribed protein (1509A/C Iso248Leu) and was associated with an increase in infarct size as well as an increase in left ventricular end-diastolic and end-systolic volumes. However, the inventors did not discover any informative mutations in the coding regions of either the A1- or A2A-AR genes. This is in contrast with multiple other G protein-coupled 7-transmembrane-spanning receptors genes which have one or more polymorphisms at sites that alter the encoded amino acids including the genes encoding the B1-, B2- and B3-adrenergic receptors, (Small K M, Annu Rev Pharmacol Toxicol 2003; 43; 381-411), alpha adrenergic receptors, (Yasuda K, Trends in Endocrinology and Metabolism 17; 269, 2006), endothelin receptors (Rossi G P, Ann NY Acad Sci 1069; 34, 2006) Activation of the A1-AR receptor during pregnancy inhibits cardiac cell proliferation and leads to cardiac hypoplasia (Zhao Z, Dev Dyn June 2001; 221:194-900). Thus, mutations that have significant impact on A1-AR gene expression during gestation might not persist in the genome because of early lethality.

The finding herein that informative SNPs were found in the 3′ untranslated region of the A1-AR gene rather than in the coding region of these genes is consistent with the recent recognition that changes in mRNA stability through binding of mRNA binding proteins including AUF1/hnRNPD, HuR, and hmRNP A1, can have profound effects on both global gene expression as well as on the levels of mRNAs and the coding of their respective proteins (Wilusz C J, Trends in Genetics 29; 491; 2004,) and can participate in the development of human disease (Chen J-M, Hum Genet. 2006; 120:1-21). For example, adrenergic agonists stimulate B-adrenergic receptor destabilization through up-regulation of several mRNA binding proteins including AUF1/hnRNP d and HuR (Blaxall B C, J Biol Chem 275; 4290; 2000, Pende A, J Biol Chem 271; 8493, 1996; Blaxall B C, Mol Cell Biochem 232; 1-11; 2002) Furthermore, both polymorphisms and insertion/deletions in the 3′UTR have been recently associated with the incidence of intracranial aneurysms (Pannu H, J Neurosurg 2006; 105; 418-23) the risk of fracture in postmenopausal women; Rivadeneira F, J Bone Miner Res 2006; 21; 1443-56; and the risk of breast cancer (Langenlehner U, Clin Cancer Res 2006; 12; 1392-4) In addition, the finding that the informative SNPs were those that were associated with a marked change in the secondary structure of the 3′ UTR region provides additional evidence that these SNPs have functional significance.

Despite substantial evidence that adenosine could mitigate at least in part the deleterious effects of ischemia-reperfusion in animal models, clinical studies of adenosine as an adjunct to reperfusion therapy in the treatment of patients with acute myocardial infarctions have provided ambiguous results. (Mahaffey K W, JACC 34:1711-20, 1999; Ross A M, JACC, 2005; 45:1775-80). The failure to demonstrate a salutary benefit of adenosine during reperfusion therapy was attributed at least in part to the use of a sub-therapeutic dose of adenosine in some patients and the need for a larger sample size. Importantly, the present invention suggests that the failure to reach adequate statistical power in recent studies may be due to the fact that the studies were enriched for patients who were less likely to respond to exogenous adenosine because of a mutation in the A1- or A3-AR gene.

The finding that informative polymorphisms in the 3′ UTR of the A1-AR gene predicted the presence of structural changes in the 3′ tail provides support for the hypothesis that these SNPs play a role in regulating A1-AR signaling. Furthermore, the data indicate that a change in the function of the A3-AR gene or a change in the stability of the A1-AR gene would alter the heart's response to myocardial ischemia.

### Example 3

Association of adenosine receptors with Baseline measures. The genotype or haplotype was assessed to investigate if they are associated with baseline measures, including BNP levels, cardiac morphology and cardiac function. Data was analyzed on several outcome variables at baseline for the participants of the STICH trial to explore the inter-relationship between the different variables. There were 786 subjects in the STICH, of whom two were omitted from analysis due to screen fail therefore leaving 766 subjects remaining for analysis.

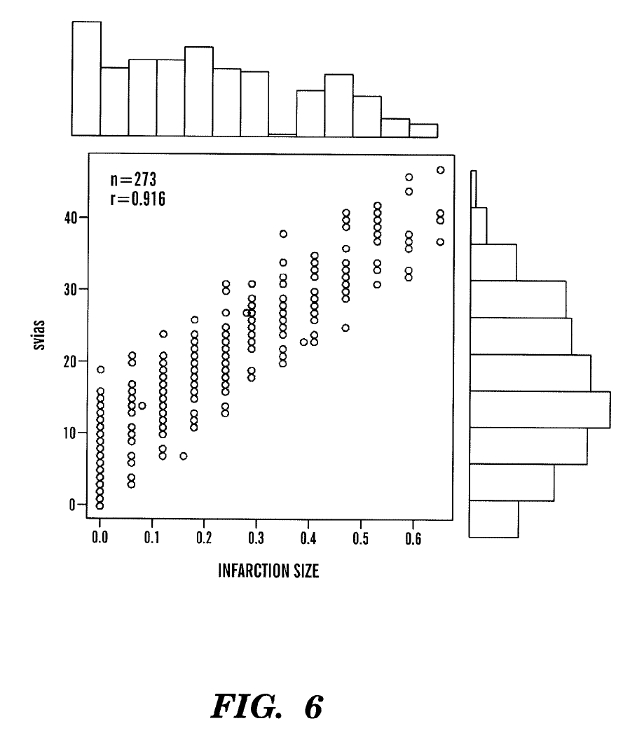

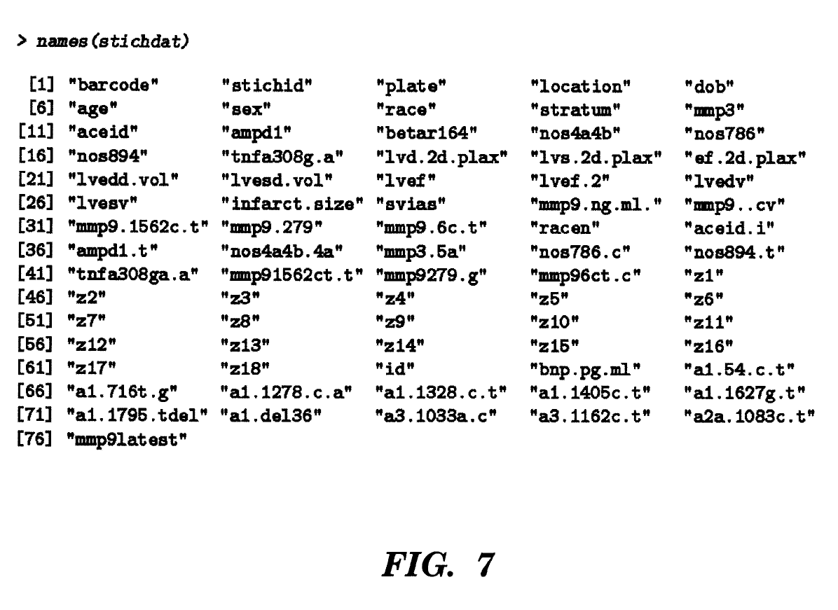

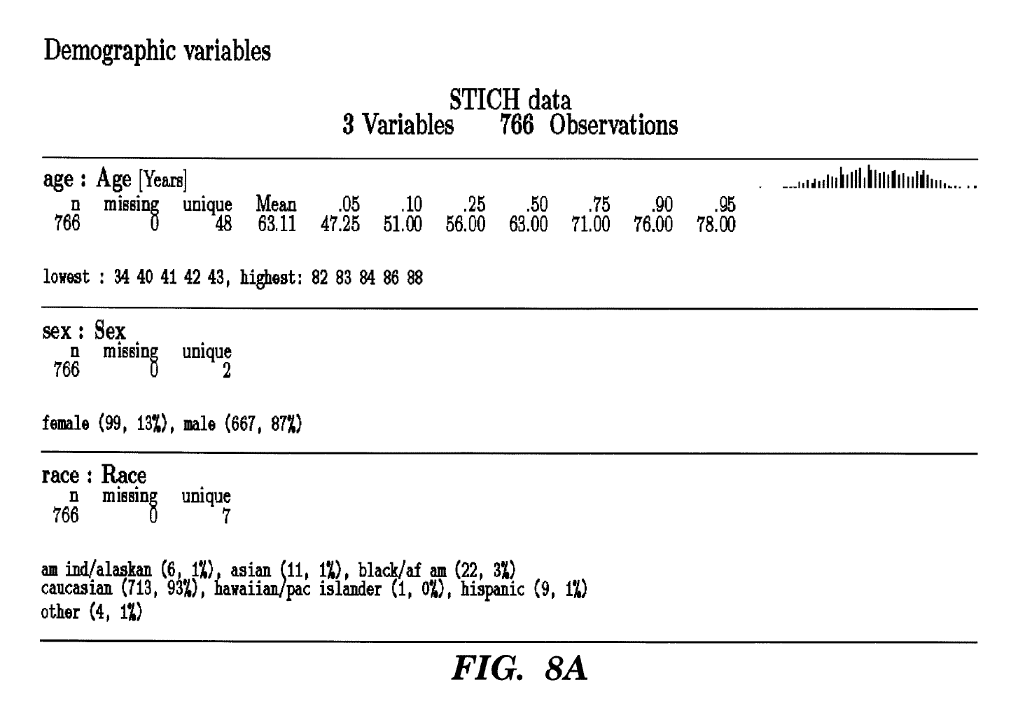

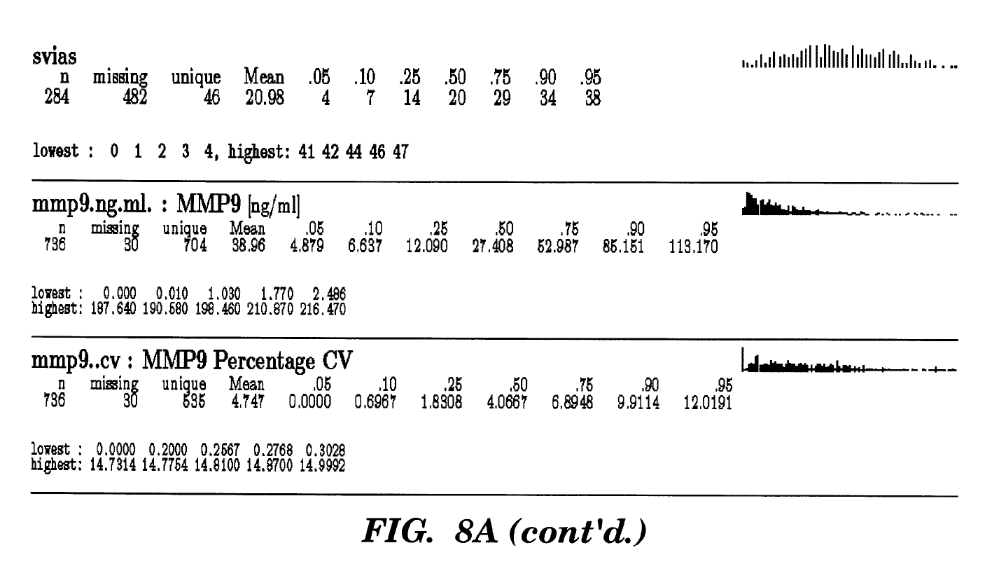

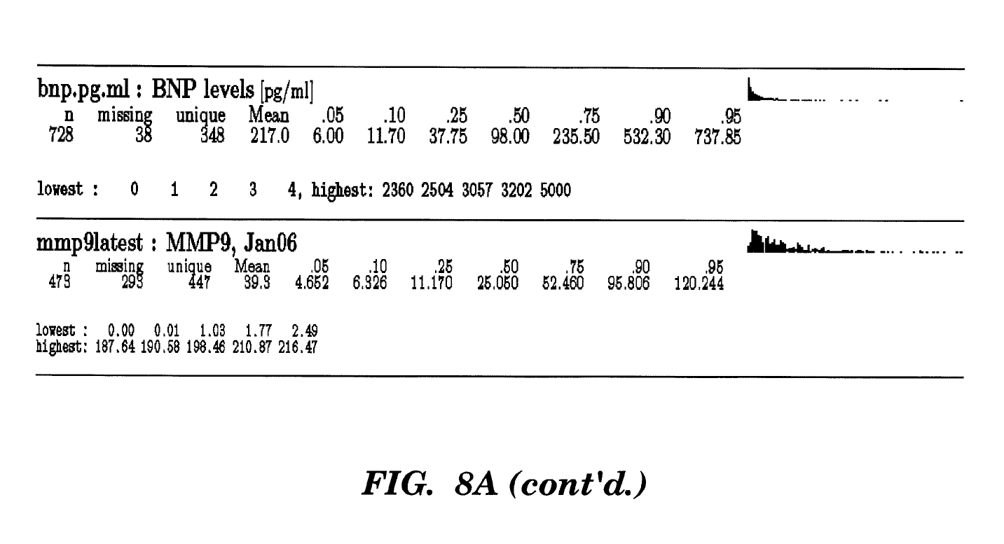

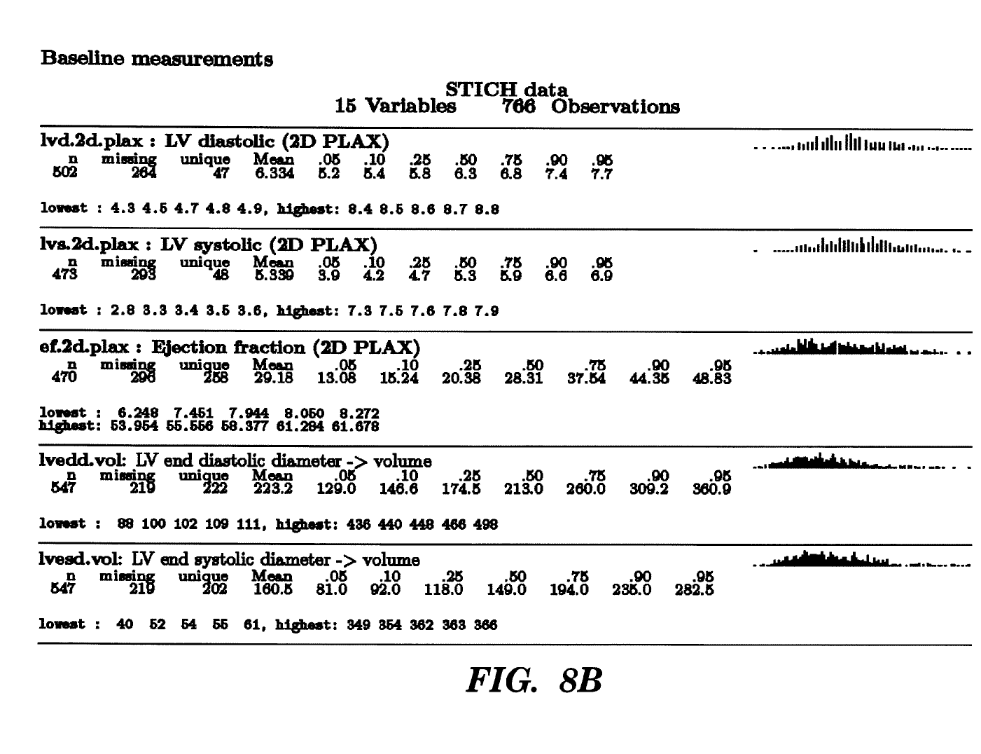

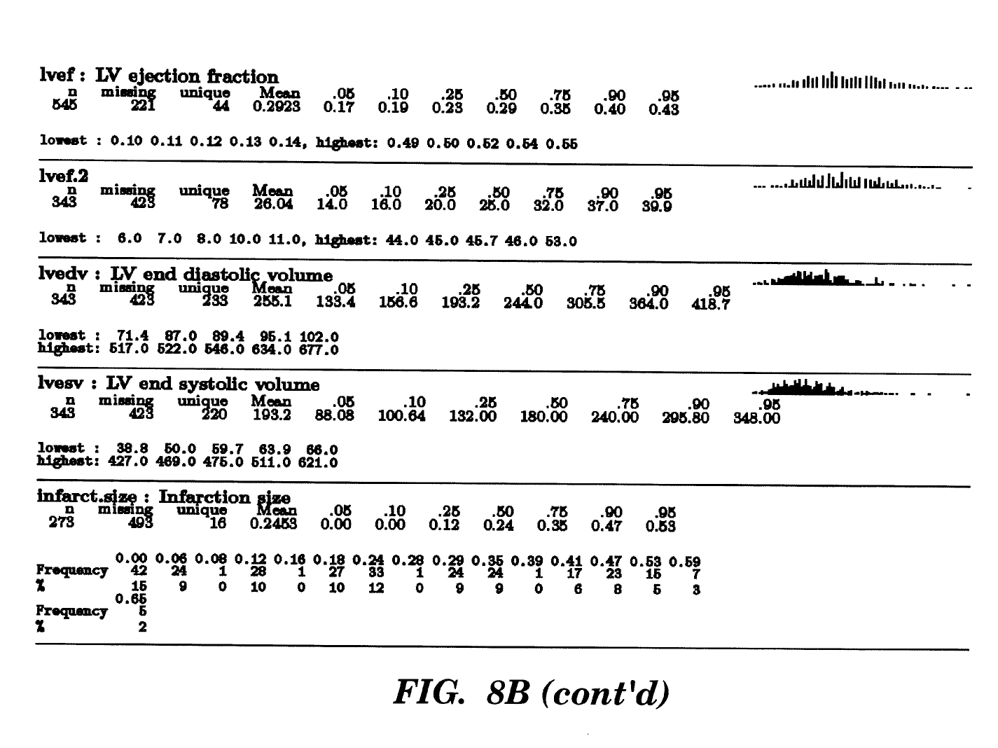

The main demographic variables are age, sex, race, with race being derived race variable and denoting either Caucasian or non-Caucasian. There are several baseline characteristics of the subjects that were recorders, related to the cardiac function or morphology, as well as B-type natriuretic peptide (BNP) and matrix metalloproteinase 9 (MMP9) levels. The baseline measurement variables are shown in Table 5 and in FIG. 7. FIGS. 8A and 8B describe the summaries of the variables collected from STICH, including the numbers missing and a small histogram in the case of numeric non-categorical variables. The STICH database contains multiple variables which measure essentially the same physiological property, for example, ejection fraction is measured by both “Ivef” and “ef.2d.plax”. We compared the variables measuring the same physiological property and assessed how well they tracked each other, shown in FIGS. 1-6. FIGS. 1-6 show that each pair has a fair degree of positive correlation, except the measures of infarction size (FIG. 6), where the “infarct.size” variable seems to be discrete in nature. Although “svias” does track with the other measure, it also exhibited a great deal of variability of each person with the same value of “infarct.size”.

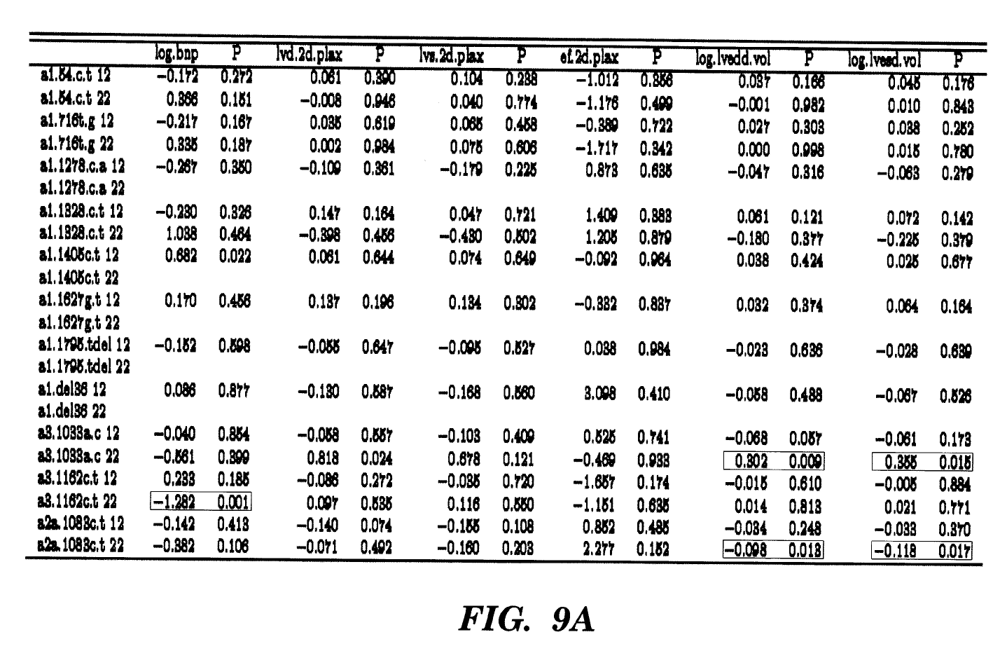

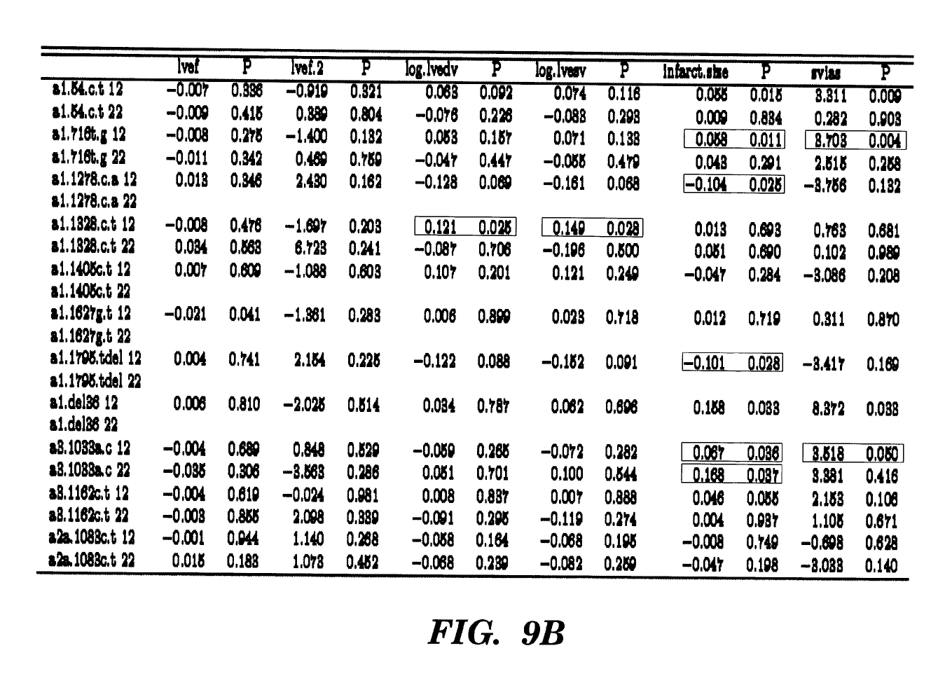

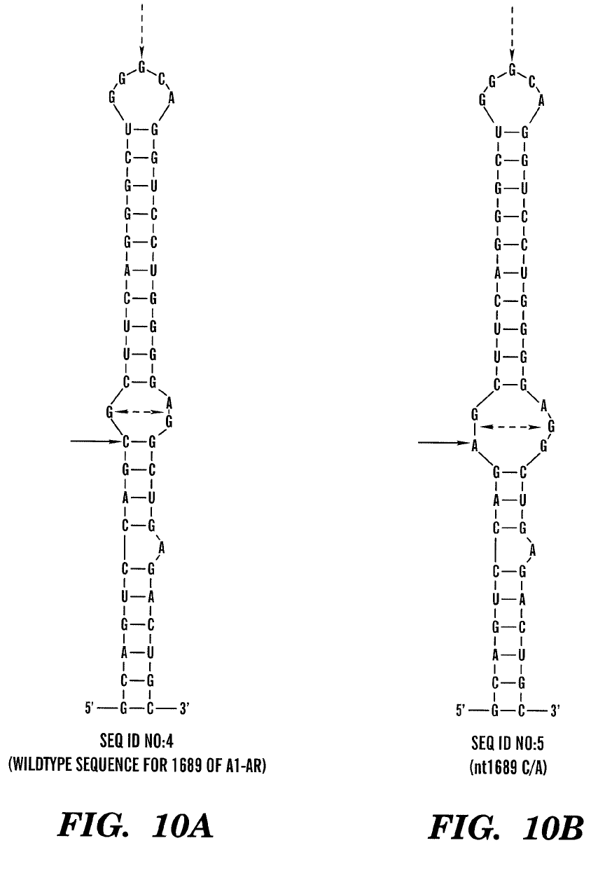

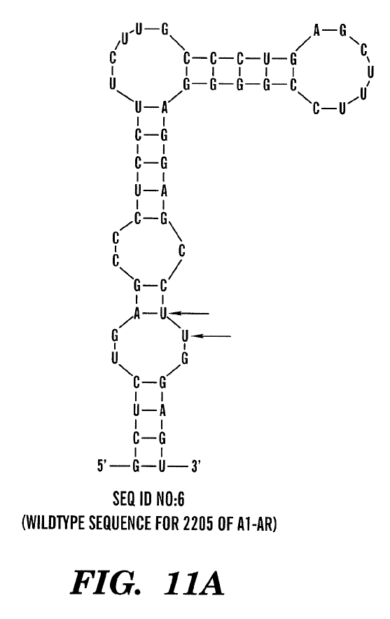

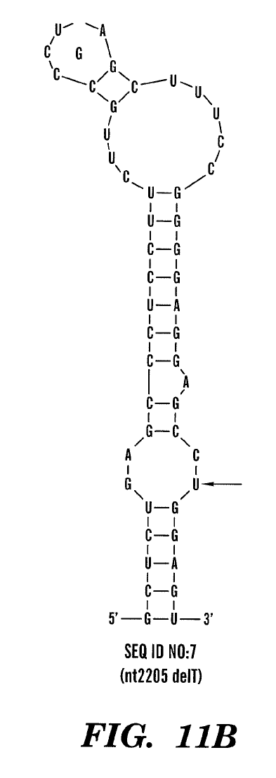

Using generalized linear model framework (Schaid et al, 2002; Am J Hum Gen, 70; 425-434) (and transforming the outcomes to the log scale as appropriate), and adjusting for age, gender and race, to assess association, we identify several statically significant variables as shown in boxed values in FIG. 9.

Also investigated were SNPs in other variables, shown in Table 6, and their percentage frequencies in the subjects enrolled in the STICH trial shown in Table 7.

TABLE 5
Baseline Measurement variables.

| | |
|---|---|
| BNP | BNP level (bnp.pg.ml) |
| Cardiac morphology | Left ventricular end systolic and diastolic diameter |
| | (lvesd.vol, lvedd.vol, lvs.2d.plax, lvd.2d.plax) |
| | Left ventricular end systolic and diastolic volume |
| | (lvedv, lvesv) |
| Cardiac function | Left ventricular ejection fraction (lvef, lvef.2, |
| | ef.2d.plax) |
| | Infarction size (infarct.size) |
| | svias |

TABLE 6
Single nucleotide polymorphisms (SNPs) collected

| | |
|---|---|
| Adenosine A1 | 54(C/T), 716(T/G), 1278(C/A), 1328(C/T), |
| | 1405(C/T), 1627(G/T), 1795(Tdel), del36 |
| Adenosine A2A | 1083(C/T) |
| Adenosine A3 | 1033(A/C), 1162(C/T) |
| TNF-α | 308(G/A) |
| NOS | 4a4b, 786(C/T), 894(G/T) |
| AMP | ampd1(C/T) |
| β-R | 164(C/T) |
| MMP3 | (5A/6A) |
| MMP9 | 1562(C/T), 279(A/C), 6(C/T) |

TABLE 7
Other SNPs.

| SNPs | N | |
|---|---|---|
| mmp3: 5A5A | 727 | 24% (172) |
| 5A6A | | 47% (341) |
| 6A6A | | 29% (214) |
| aceid: DD | 763 | 28% (212) |
| ID | | 52% (395) |
| II | | 20% (155) |
| IO | | 0% (1) |
| ampd1: CC | 761 | 71% (544) |
| CT | | 27% (203) |
| TT | | 2% (14) |
| betar164: CC | 765 | 98% (746) |
| CT | | 2% (19) |
| nos4a4b: 4a4a | 763 | 2% (17) |
| 4b4a | | 28% (217) |
| 4b4b | | 69% (529) |
| nos786: CC | 762 | 15% (115) |
| TC | | 47% (355) |
| TT | | 38% (292) |
| tnfa308g.a: AA | 762 | 2% (17) |
| GA | | 26% (198) |
| GG | | 72% (547) |

N is the number of non-missing values.
Numbers are after percents are frequencies.

### Example 4

Haplotype-Trait Association. The inventors also assessed if any of the inferred haplotypes are associated with the baseline characteristics of the patients. This was done using generalized linear model framework (Schaid et al, 2002; Am J Hum Gen, 70; 425-434) and adjusting for age, sex and race. These were fitted to a model assuming normally distributed data, and log-transforming the outcome of the data (data not shown).

To assess whether the presence of a genetic variant was associated with the development of myocardial ischemia and left ventricular dysfunction we assessed the allele frequency of the common variants in 273 patients with ischemic cardiomyopathy and in a population of 203 normal controls with no history of cardiovascular disease. In the ischemic heart failure population, 96% were Caucasian, 11%, were women and the mean age was 62. In the controls, all were Caucasian, 39% were women and the mean age was 60. Eighty-five percent of the ischemia cardiomypathy patients analyzed had a demonstrable infract on nuclear imaging. As shown in Table 8, the allele frequency for variants in the A1-AR gene did not differ significantly amongst the two groups, however, there was a statistically higher frequency of genetic variants for the A3-AR in the population with ischemic heart disease than in the normal controls.

As shown in Table 9, when adjusted for age, sex, and race, the presence of a single allelic SNP at nt 1689 C/A or a deletion at nt 2206 Tdel in the 3′ UTR of the A1-AR gene was associated with a decrease in infarct size whereas a 36 nt deletion at nt 2683 in the 3′UTR of the A1-AR gene was associated with an increase in infarct size. No patients harbored a variant at any of these three sites on both alleles and the prevalence of even the heterozygous condition was small. The inventors determined the variants in the A1-AR gene were in Hardy-Weinberg, disequilibrium (d not shown). When analyzed using the mfold program, the inventors discovered all three of these variants in the 37 UTR of the A1-AR resulted in a significant change in the secondary structure of the mRNA (1689(C/A: FIG. 1A; 2206′ Tdel: FIG. 1B; 2683del36: data not shown). There was also a significant association between an increase in infarct size and a polymorphism in a single allele at either nt −54+C/T or in a SNP within the coding region at nit 717 T/G; however, an effect was not obvious ion infarct size when a single allele in the case of nt 717 T/G or both alleles in the case of nt −54+C/T harbored the mutation suggesting that these SNPs were not informative. However, the presence of a relatively common SNP on either one or both alleles in the coding region of the A3-AR gene at nt 1509 A/C (Iso248Leu) was associated with an increase 11n infarct size. (Table 9). This polymorphism or sequence difference was the only one which was discovered by the inventor in all of the AR genes that effected a change in an amino acid.

TABLE 8
Frequency of Genetic Variants by Group
Frequency of Genetic Variants by Group

| | | | | p value/ |
|---|---|---|---|---|
| | | Control | STICH | adjusted p- |
| SNP | | (203) | (273) | value |
| A₁ −54 c/t | +/− | 45.2 | 45.4 | 0.54/0.99 |
| | +/− | 44.1 | 46.2 | |
| | −/− | 10.7 | 7.7 | |
| A₁ 717 t/g | +/− | 41.6 | 44.7 | 0.48/0.99 |
| | +/− | 46.7 | 46.2 | |
| | −/− | 11.7 | 8.4 | |
| A₁ 1,689 c/a | +/− | 93.6 | 93.4 | 0.81/1.00 |
| | +/− | 6.4 | 5.9 | |
| | −/− | 0 | 0 | |
| A₁ 1,739 c/t | +/− | 87.0 | 86.1 | 0.48/0.99 |
| | +/− | 13.0 | 12.8 | |
| | −/− | 0 | 0.7 | |
| A₁ 1,816 c/t | +/− | | 91.9 | — |
| | +/− | | 7.0 | |
| | −/− | | 0 | |
| A₁ 2,838 g/t | +/− | 90.8 | 86.8 | 0.30/0.97 |
| | +/− | 9.1 | 12.1 | |
| | −/− | 0 | 0 | |
| A₁ 2,206 Tdel | +/− | 93.3 | 93.0 | 0.74/1.00 |
| | +/− | 6.7 | 5.9 | |
| | −/− | 0 | 0 | |
| A₁ 2,683 del 36 | +/− | 97.0 | 97.4 | 0.60/0.99 |
| | +/− | 3.0 | 2.2 | |
| | −/− | 0 | 0 | |
| A₃ 1,509 a/c | +/− | 95.8 | 81.4 | 1.2 × 10⁻⁵/<0.001 |
| | +/− | 4.2 | 16.4 | |
| | −/− | 0 | 2.2 | |
| A₃ 1,664 c/t | +/− | 80.9 | 60.6 | 1.9 × 10⁻⁶/<0.001 |
| | +/− | 12.8 | 32.9 | |
| | −/− | 6.0 | 6.4 | |

TABLE 9
Association of Adenosine receptor SNP genotypes with infarct size.

| | Genotype | | | | | | | | |
|---|---|---|---|---|---|---|---|---|---|
| | (+/+) | | (+/−) | | (−/−) | | | Effect 1 | Effect 2 |
| Adenosine | Baseline | | Mean 1 | | Mean 2 | | Type 3 | Mean 1 - | Mean 2 - |
| receptor | infarct | | infarct | | infarct | | Overall P | Baseline * | Baseline * |
| SNP | size | n | size * | n | size * | n | value | (P value) | (P value) |
| A1 54 C/T | .220 | 124 | 0.274 | 126 | 0.220 | 21 | 0.076 | 0.052 | 0.019 |
| | | | | | | | | (0.024) | (0.664) |
| A1 717 T/G | .215 | 122 | 0.273 | 126 | 0.251 | 23 | 0.044 | 0.056 | 0.045 |
| | | | | | | | | (0.014) | (0.278) |
| A1 1689 C/A | .251 | 255 | 0.151 | 16 | 0.251 | 255 | 0.021 | −0.107 | |
| | | | | | | | | (0.021) | |
| A1 1739 C/T | .242 | 235 | 0.255 | 35 | 0.295 | 2 | 0.859 | 0.013 | 0.051 |
| | | | | | | | | (0.692) | (0.692) |
| A1 1816 C/T | .249 | 251 | 0.196 | 19 | 0.249 | 251 | 0.364 | −0.040 | |
| | | | | | | | | (0.364) | |
| A1 2038 G/T | .245 | 237 | 0.246 | 33 | 0.245 | 237 | 0.519 | 0.023 | |
| | | | | | | | | (0.519) | |
| A1 2206 Tdel | .250 | 254 | 0.151 | 16 | 0.250 | 254 | 0.024 | −0.104 | |
| | | | | | | | | (0.024) | |
| A1 2683 del36 | .241 | 266 | 0.393 | 6 | 0.241 | 266 | 0.033 | 0.158 | |
| | | | | | | | | (0.033) | |
| A3 1509 A/C | .221 | 184 | 0.291 | 37 | 0.388 | 5 | 0.012 | 0.073 | 0.169 |
| | | | | | | | | (0.024) | (0.035) |
| A3 1664 C/T | .229 | 160 | 0.275 | 87 | 0.229 | 17 | 0.207 | 0.042 | −0.000 |
| | | | | | | | | (0.081) | (0.996) |

In the headers, + refers to the major allele and − to the minor allele. For each available genotype, the adjusted least squares mean infarct size is given along with number of observations. The effect given for each genotype is the difference in infarct size with the (+/+) genotype as estimated using linear regression, adjusted for age, race and sex, with p-value given in parentheses. The sequence differences which correspond to a change in responsiveness to adenosine agonist treatment and/or predict relative infarct size as disclosed herein are highlighted in bold.

Thus the inventors have demonstrated an association between polymorphisms or sequence differences in the A1- and A3-adenosine genes and infarct size in a population of patients with a history of coronary artery disease and left ventricular dysfunction and an increase in the frequency of genetic variants in the A3-AR in this same population.

The inventors have also demonstrated a single polymorphism or sequence difference in the A3-AR gene that resulted in a change in the amino acid sequence of the transcribed protein (1509 a/c Iso248Leu) and was associated with an increase in infarct size. Activation of the A1-AR receptor during pregnancy inhibits fetal cardiac cell proliferation and leads to cardiac hypoplasia. Thus, the inventors discovery that the sequence differences that have significant impact on A1-AR gene expression during gestation might not persist in the genome because of early lethality.

The inventors have discovered informative variants and sequence differences in the 3′UTR of the A1-AR gene rather than in the coding region of these genes leads to changes in mRNA stability through binding of mRNA binding proteins. Such alteration of mRNA stability can have profound effects on both global gene expression as well as on the levels of mRNAs and the coding of their respective proteins and can participate in the development of human disease.11 For example, adrenergic agonists stimulate β-adrenergic receptor destabilization through up-regulation of several mRNA binding proteins and both polymorphisms and insertion/deletions in the 3′ UTR have been recently associated with human disease.28-30 The inventors discovered that all three informative sequence differences or variants in the A1-AR gene were found to be associated with a marked change in the secondary structure of the 3′ UTR.

The utility of the RNA Mfold for predicting mRNA folding has been recently confirmed by analysis of the structural effects of polymorphisms in the catechol-O-methyltransferase gene.31 As disclosed herein, the inventors discovered that the 1689 C/A polymorphism involved the replacement of a 1×2 internal loop (allele C) by a 2×3 internal loop (allele A). Both the two internal loops and the hairpin loop contain AG-rich sequences similar to those previously demonstrated to be an important class of cis-regulatory elements in the 3′ UTRs of protein-coding genes.11 Similarly, the inventors discovered 2206 Tdel variant caused a significant change in RNA secondary structure that is classified as a Pattern I secondary structure change in accordance with Chen et al.11 which is a reliable indicator of functionality, based upon an analysis of experimentally characterized 3′ UTR variants. The inventors also demonstrate the deletion of 36 nucleotides at position 2683del36 resulted in a significant change in secondary structure which affects different stages of post-transcriptional gene regulation. Thus, the inventors have discovered that the three disease-associated variants were associated with alterations in secondary structure demonstrates that these variants have functional significance.

Sequences.

Nucleic acid transcript sequence for A1-AR which refers to Ensemble ID: ENSG00000163485 and corresponds to SEQ ID NO: 1 herein is shown as the top nucleic acid sequence numbered from 1 to 2896. SEQ ID NO: 1 corresponds to the wild type (WT) A1-AR nucleic acid sequence. The translation of the nucleic acid sequence into the amino acid sequence is shown below the nucleic acid sequence, and corresponds to SEQ ID NO: 4. Also shown are the variances in A1-AR nucleic acid sequence SEQ ID NO:1 which correspond to the polymorphisms in the A1-AR nucleic acid sequence as disclosed herein, for example, the label “nt1689(1278)C/A(rs6427994)” refers to the substitution of an A with a C at position 1689 in SEQ ID NO:1. The reference in “( )” refers to RefSNP # or dbSNP rs#, for example the rs6427994 identifies the polymorphism in the nt1689(1278)C/A in SEQ ID NO:1.

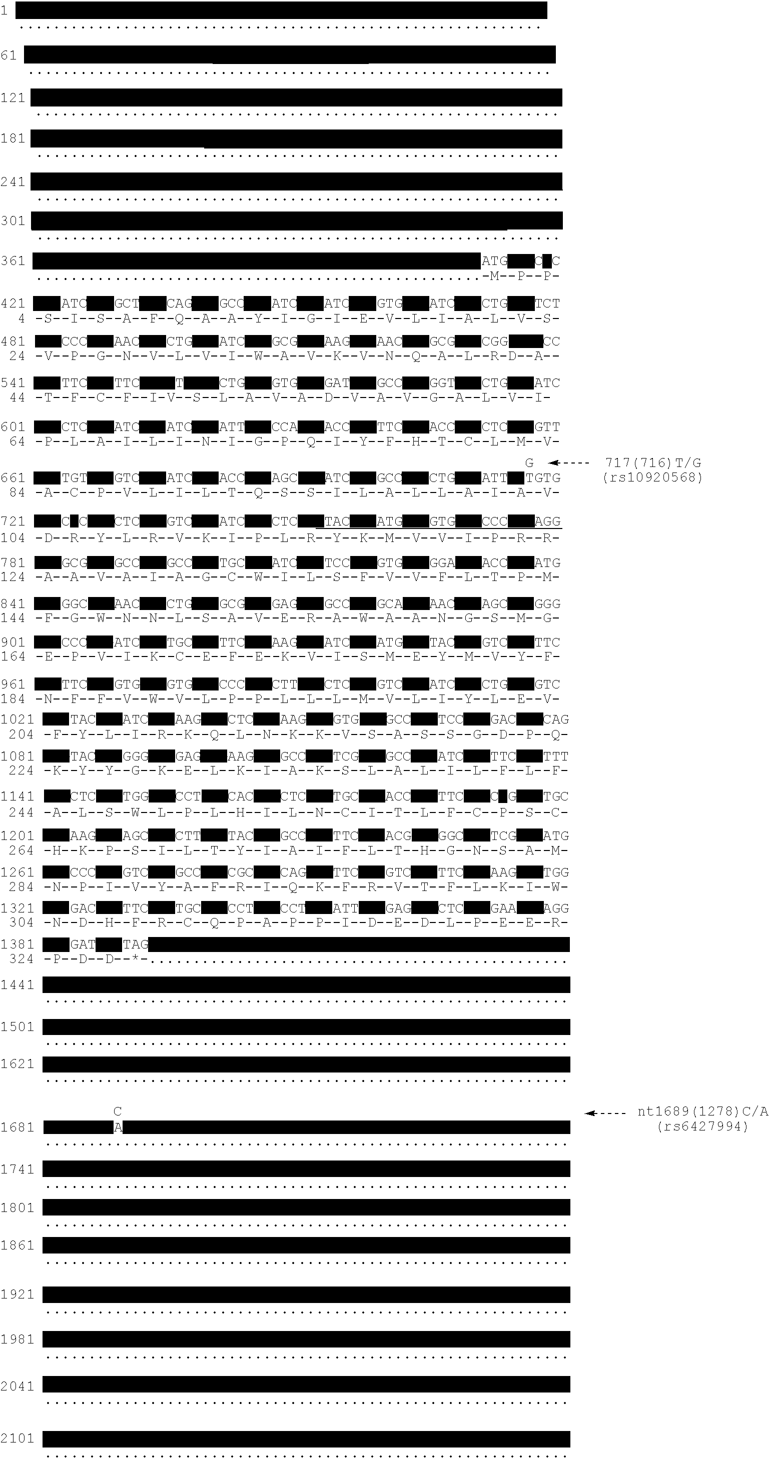

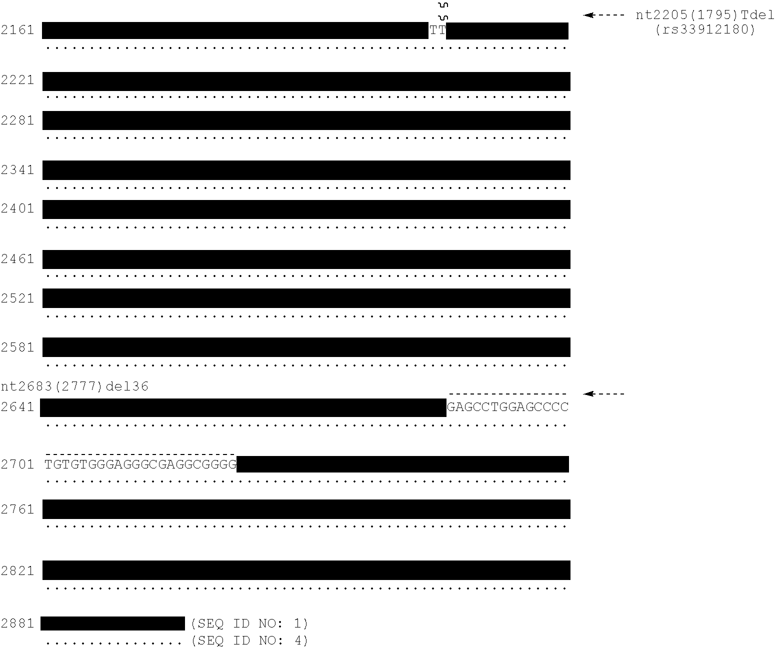

Nucleic acid transcript sequence for A3-AR which refers to Ensemble ID: ENST000002141356 and corresponds to SEQ ID NO:2 herein is shown as the top nucleic acid sequence numbered from 1 to 2241. SEQ ID NO: 2 corresponds to the wild type (WT) A3-AR nucleic acid sequence. The translation of the nucleic acid sequence into the amino acid sequence is shown below the nucleic acid sequence, and corresponds to SEQ ID NO:3 or GenBank No: NP_{—}000668. Also shown are the variances in A3-AR nucleic acid sequence SEQ ID NO:2 which correspond to the polymorphisms in the A3-AR nucleic acid sequence as disclosed herein, for example, the label “nt1509(1033)A/C(rs35511654)” refers to the substitution of an A with a C at position 1509 in SEQ ID NO:2. The reference in “( )” refers to RefSNP # or dbSNP rs#, for example the rs35511654 identifies the polymorphism in nt1509(1033)A/C in SEQ ID NO:2.

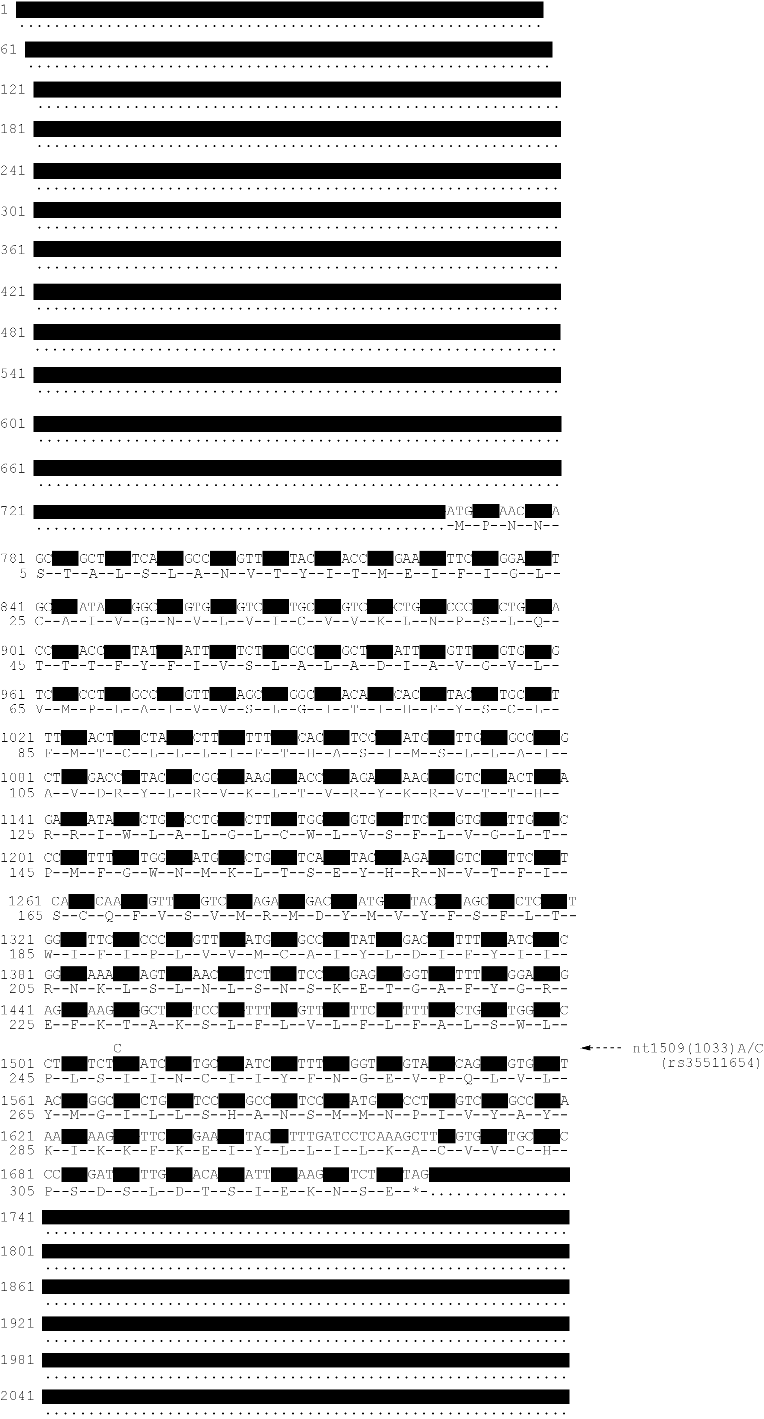

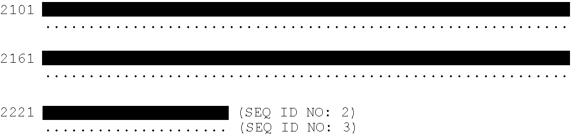

| SEQ ID NO: 3 NP_000668 | |
|---|---|
| 1 | mpnnstalsl anvtyitmei figlcaivgn vlvicvvkln pslqtttfyf ivslaladia |
| 61 | vgvlvmplai vvslgitihf ysclfmtcll lifthasims llaiavdryl rvkltvrykr |
| 121 | vtthrriwla lglcwlvsfl vgltpmfgwn mkltseyhrn vtflscqfvs vmrmdymvyf |
| 181 | sfltwifipl vvmcaiyldi fyiirnklsl nlsnsketga fygrefktak slflvlflfa |
| 241 | lswlplsiin ciiyfngevp qlvlymgill shansmmnpi vyaykikkfk etyllilkac |
| 301 | vvchpsdsld tsieknse |

### REFERENCES

The references cited herein and throughout the application are incorporated herein by reference.
1. Headrick J P, Hack B, Ashton K J. Acute adenosinergic cardioprotection in ischemic-reperfused hearts. Am J Physiol Heart Circ Physiol. November 2003; 285(5):H1797-1818.
2. Reichelt M E, Willems L, Molina J G, et al. Genetic deletion of the A1 adenosine receptor limits myocardial ischemic tolerance. Circ Res. Feb. 18 2005; 96(3):363-367.
3. Matherne G P L J, Byford A M, Gauthier N S, Headrick J P. Transgenic A1 adenosine receptor overexpression receptor increases myocardial resistance to ischemia. Proc Natl Acad Sci USA. 1997(94):6541-6546.
4. Black R G Jr. G Y, Ge Z D, Murphee S S, Prabhu S D, Jones W K, Bolli R, Auchampach J A. Gene dosage-dependent effects of cardiac-specific overexpression of the A3 adenosine receptor. Circ Res. 2002; 91:165-172.
5. Yang Z, Cerniway R J, Byford A M, et al. Cardiac overexpression of A1-adenosine receptor protects intact mice against myocardial infarction. Am J Physiol Heart Circ Physiol. March 2002; 282(3):H949-955.
6. Liggett S B, Mialet-Perez J, Thaneemit-Chen S, et al. A polymorphism within a conserved beta(1)-adrenergic receptor motif alters cardiac function and beta-blocker response in human heart failure. Proc Natl Acad Sci USA. Jul. 25 2006; 103(30):11288-11293.
7. Alsene K, Deckert J, Sand P, et al. Association between A2a receptor gene polymorphisms and caffeine-induced anxiety. Neuropsychopharmacology. September 2003; 28(9):1694-1702.
8. Deckert J, Nothen M M, Franke P, et al. Systematic mutation screening and association study of the A1 and A2a adenosine receptor genes in panic disorder suggest a contribution of the A2a gene to the development of disease. Mol. Psychiatry. January 1998; 3(1):81-85.
9. Hamilton S P, Slager S L, De Leon A B, et al. Evidence for genetic linkage between a polymorphism in the adenosine 2A receptor and panic disorder. Neuropsychopharmacology. March 2004; 29(3):558-565.
10. Velazquez E J L K, O'Connor C M, Oh J K, Bonow R O, Pohost G M, Feldman A M, Mark D B, Panza J A, Sopko G, Rouleau J., Jones R H for the STITCH Investigators. The Rationale and Design of the Surgical Treatment for Ischemic Failure (STITCH) Trial. J of Thoracic and Cardiovascular Surgery. in press.
11. Chen J M, Ferec C, Cooper D N. A systematic analysis of disease-associated variants in the 3′ regulatory regions of human protein-coding genes II: the importance of mRNA secondary structure in assessing the functionality of 3′ UTR variants. Hum Genet. October 2006; 120(3):301-333.
12. Zuker M. Mfold web server for nucleic acid folding and hybridization prediction. Nucleic Acids Res. Jul. 1 2003; 31(13):3406-3415.
13. Dilsizian V, Rocco T P, Freedman N M, et al. Enhanced detection of ischemic but viable myocardium by the reinjection of thallium after stress-redistribution imaging. N Engl J Med. Jul. 19 1990; 323(3):141-146.
14. Sciagra R, Bisi G, Santoro G M, et al. Comparison of baseline-nitrate technetium-99m sestamibi with rest-redistribution thallium-201 tomography in detecting viable hibernating myocardium and predicting postrevascularization recovery. J Am Coll Cardiol. August 1997; 30(2):384-391.
15. Perrone-Filardi P, Pace L, Prastaro M, et al. Assessment of myocardial viability in patients with chronic coronary artery disease. Rest-4-hour-24-hour 201T1 tomography versus dobutamine echocardiography. Circulation. Dec. 1 1996; 94(11):2712-2719.
16. Sidak Z. Rectangular confidence regions for the means of miltivariate normal distributions J of the American Statistical Association 1977; 62:626-633.
17. Leesar M A, Stoddard M, Ahmed M, et al. Preconditioning of human myocardium with adenosine during coronary angioplasty. Circulation. Jun. 3 1997; 95(11):2500-2507.
18. Small K M, McGraw D W, Liggett S B. Pharmacology and physiology of human adrenergic receptor polymorphisms. Annu Rev Pharmacol Toxicol. 2003; 43:381-411.
19. Yasuda K, Matsunaga T, Adachi T, et al. Adrenergic receptor polymorphisms and autonomic nervous system function in human obesity. Trends Endocrinol Metab. September 2006; 17(7):269-275.
20. Rossi G P, Pitter G. Genetic variation in the endothelin system: do polymorphisms affect the therapeutic strategies? Ann N Y Acad Sci. June 2006; 1069:34-50.
21. Zhao Z, Rivkees S A. Inhibition of cell proliferation in the embryonic myocardium by A1 adenosine receptor activation. Dev Dyn. June 2001; 221(2):194-200.
22. Deckert J, Nothen M M, Albus M, et al. Adenosine A1 receptor and bipolar affective disorder: systematic screening of the gene and association studies. Am J Med Genet. Feb. 7 1998; 81(1):18-23.
23. Iida A, Saito S, Sekine A, et al. Catalog of 300 SNPs in 23 genes encoding G-protein coupled receptors. J Hum Genet. 2004; 49(4):194-208.
24. Wilusz C J, Wilusz J. Bringing the role of mRNA decay in the control of gene expression into focus. Trends Genet. October 2004; 20(10):491-497.
25. Blaxall B C, Pellett A C, Wu S C, et al. Purification and characterization of beta-adrenergic receptor mRNA-binding proteins. J Biol Chem. Feb. 11 2000; 275(6):4290-4297.
26. Pende A, Tremmel K D, DeMaria C T, et al. Regulation of the mRNA-binding protein AUF1 by activation of the beta-adrenergic receptor signal transduction pathway. J Biol Chem. Apr. 5 1996; 271(14):8493-8501.
27. Blaxall B C, Pende A, Wu S C, et al. Correlation between intrinsic mRNA stability and the affinity of AUF1 (hnRNP D) and HuR for A+U-rich mRNAs. Mol Cell Biochem. March 2002; 232(1-2):1-11.
28. Pannu H, Kim D H, Guo D, et al. The role of MMP-2 and MMP-9 polymorphisms in sporadic intracranial aneurysms. J Neurosurg. September 2006; 105(3):418-423.
29. Rivadeneira F, van Meurs J B, Kant J, et al. Estrogen receptor beta (ESR2) polymorphisms in interaction with estrogen receptor alpha (ESR1) and insulin-like growth factor I (IGF1) variants influence the risk of fracture in postmenopausal women. J Bone Miner Res. September 2006; 21(9):1443-1456.
30. Langsenlehner U, Yazdani-Biuki B, Eder T, et al. The cyclooxygenase-2 (PTGS2) 8473T>C polymorphism is associated with breast cancer risk. Clin Cancer Res. Feb. 15 2006; 12(4):1392-1394.
31. Nackley A G, Shabalina S A, Tchivileva I E, et al. Human catechol-O-methyltransferase haplotypes modulate protein expression by altering mRNA secondary structure. Science. Dec. 22 2006; 314(5807):1930-1933.

1. A method for predicting whether a subject will be responsive to an adenosine agonist treatment, the method comprising:
analyzing a sample comprising nucleic acid from a subject for the presence of a sequence difference in the 3′-untranslated region (3′-UTR) of the A1 adenosine receptor gene relative to the 3′-UTR of SEQ ID NO:1 using real time PCR,
wherein the sequence difference in the 3′-UTR affects the stability of the adenosine receptor A1 RNA as compared with the stability of adenosine receptor A1 RNA corresponding to SEQ ID NO:1 using real time PCR,
wherein a sequence difference that increases the stability of the adenosine receptor A1 RNA relative to the stability of adenosine receptor A1 RNA corresponding to SEQ ID NO:1 identifies a subject with a likelihood of decreased responsiveness to an adenosine agonist treatment, and
wherein a sequence difference that decreases the stability of the adenosine receptor A1 RNA relative to the stability of adenosine receptor A1 RNA corresponding to SEQ ID NO:1 identifies a subject with a likelihood of increased responsiveness to an adenosine agonist treatment, and
wherein if there is no sequence difference in the 3′UTR of the A1 adenosine receptor RNA corresponding to SEQ ID NO:1, the subject is identified as being likely to be responsive to an adenosine agonist treatment.

2. The method of claim 1, wherein the sequence difference that identifies a subject with a likelihood of a decreased responsiveness to adenosine agonist treatment is selected from at least one of (i) a change of a cytosine (C) in the 3′UTR of the A1 adenosine receptor gene at position 1689 of SEQ ID NO:1 to an adenosine (A) (nt1689(1278)C/A), (ii) a deletion of a thymidine (T) in the 3′UTR of the A1 adenosine receptor gene at position 2205 of SEQ ID NO:1 (nt2205(1790)delT).

3. (canceled)

4. The method of claim 1, wherein the sequence difference that identifies an subject with a likelihood of an increased responsiveness to adenosine agonist treatment is selected from at least one of (i) a deletion of at least 1 nucleotides in the 3′UTR of the A1 adenosine receptor gene between position 2683 and 2719 of SEQ ID NO:1 or (ii) a deletion of 36 nucleotide in the 3′UTR of the A1 adenosine receptor gene beginning at position 2683 of SEQ ID NO:1 (nt2683(2777)del36).

5.-7. (canceled)

8. The method of claim 1, further comprising administering an adenosine agonist treatment to a subject if the subject is identified to have a likelihood of an increased responsiveness to adenosine agonist treatment or identified to be likely to be responsive to an adenosine agonist treatment.

9. (canceled)

10. The method of claim 1, further comprising administering an appropriate non-adenosine agonist treatment to the subject if the subject is identified to have a likelihood of decreased responsiveness to adenosine agonist treatment.

11. A method for predicting whether a subject will be responsive to an adenosine agonist treatment, the method comprising:
analyzing a sample comprising nucleic acid from a subject for the presence of a sequence difference in the nucleic acid sequence encoding the A3 adenosine receptor gene as compared to the nucleic acid sequence corresponding to SEQ ID NO:2 using real time PCR,
wherein the sequence difference in the nucleic acid sequence affects a function of the A3 adenosine receptor protein as compared with that function of the A3 adenosine receptor protein corresponding to an A3 adenosine receptor having the amino acid sequence of SEQ ID NO:3 using real time PCR,
wherein a sequence difference that decreases the function of the A3 adenosine receptor protein relative to the function of the A3 adenosine receptor protein corresponding to an A3 adenosine receptor having amino acid sequence of SEQ ID NO:3 identifies a subject with a likelihood of increased responsiveness to an adenosine agonist treatment relative to a subject with A3 adenosine receptor of SEQ ID NO:3, and
wherein if there is no sequence difference in the amino acid sequence of the A3 adenosine receptor corresponding to SEQ ID NO:3, the subject is identified as being likely to be responsive to an adenosine agonist treatment.

12. (canceled)

13. (canceled)

14. The method of claim 11, wherein the sequence difference in the nucleic acid encoding A3 adenosine receptor changes the identity of amino acid number 248 of the human A3 adenosine receptor gene corresponding to SEQ ID NO:3.

15. The method of claim 11, wherein the sequence difference in the nucleic acid encoding A3 adenosine receptor changes an Isoleucine to a Leucine at amino acid 248 of SEQ ID NO:3. (1248L)

16. The method of claim 11, wherein the sequence difference in the nucleic acid encoding A3 adenosine receptor is a change of an adenosine (A) to a cytosine (C) at the nucleotide corresponding to position 1509 of the nucleic acid corresponding to SEQ ID NO:2 encoding the A3 adenosine receptor gene. (nt1509(1033)A/C)

17. (canceled)

18. The method of claim 11, further comprising administering an adenosine agonist treatment to the subject if the subject is identified to have a sequence difference that results in an increased responsiveness to an adenosine agonist treatment.

19.-47. (canceled)

48. A computer based platform to compare input data for a method off directing treatment in a subject, wherein the computer based platform compares at least one of;
a sequence difference in the A1 adenosine receptor 3′UTR as compared to the nucleic acid sequence corresponding to SEQ ID NO:1 in a biological sample obtained from the subject, and/or
a sequence difference in the A3 adenosine receptor gene as compared to the nucleic acid corresponding to SEQ ID NO:2 in a biological sample obtained from the subject,
wherein if the computer based platform comparison identifies a sequence difference in the 3′UTR of the A1 adenosine receptor gene which corresponds to a deletion of at least one nucleic acid beginning at position 2683 of SEQ ID NO:1, and/or a sequence difference in the A3 adenosine receptor gene which corresponds to a change in 1509(1033)A/C of SEQ ID NO:2, the computer platform presents information identifying the subject has an increased likelihood for responsiveness to an adenosine agonist treatment and a clinician directs the subject to be treated with an appropriate adenosine agonist treatment, and
wherein if the computer based platform comparison identifies a sequence difference in the 3′UTR of the A1 adenosine receptor gene which corresponds to a change in 1698(1278)C/A of SEQ ID NO:1, and/or a change in 2205(1790)Tdel of SEQ ID NO:1, the computer platform presents information identifying the subject has the likelihood of decreased responsiveness to an adenosine agonist treatment, and a clinician directs the subject to be treated with an appropriate treatment other than an adenosine agonist treatment.

49. (canceled)

50. A kit comprising at least one probe to specifically detect a sequence difference in at least one of a nucleotide sequence SEQ ID NO: 1 or SEQ ID NO: 2 or the amino acid of SEQ ID NO: 3, wherein the sequence difference in SEQ ID NO: 1 is selected from the group of nt1689(1278)C/A, nt2205(1790)delT, at least one nucleic acid difference beginning at position 2683, or nt2683(2777)del36; wherein the sequence difference in SEQ ID NO: 2 is nt1509(1033)A/C; and wherein the sequence difference in SEQ ID NO; 3 is 1248L.

51.-56. (canceled)

57. The kit of claim 50, wherein the probe comprises a nucleic acid, nucleic acid analogue, a protein, polypeptide, antibody, antibody fragment, humanized antibody, chimeric antibody, recombinant protein, recombinant antibody, small molecule, aptamer, protein aptamer and variant or fragment thereof.

58.-60. (canceled)

61. The computer platform of claim 48, wherein the method to direct the treatment in a subject is preventing or reducing the risk of a subject with a myocardial infarction wherein if the computer based platform comparison identifies a sequence difference in the 3′UTR of the A1 adenosine receptor gene which corresponds to a deletion of at least one nucleic acid beginning at position 2683 of SEQ ID NO:1, and/or a sequence difference in the A3 adenosine receptor gene which corresponds to a change in 1509(1033)A/C of SEQ ID NO:2, the computer platform presents information identifying the subject has an increased likelihood for having a large infarct size and a clinician directs the subject to be treated with an appropriate adenosine agonist treatment.

62. The computer platform of claim 48, wherein the computer platform comprises a database comprising information of the sequence information of at least one of the A1 or A3 adenosine receptor genes, and optionally clinical status of the tissue sample from which the sequence information of the adenosine receptor was derived.
